(19) United States
(12) Patent Application Publication
(10) Pub. No.: US 2025/0186536 A1
(43) Pub. Date: Jun. 12, 2025

(54) SUSTAINED RELEASE PEPTIDE FORMULATIONS

(71) Applicant: RHYTHM PHARMACEUTICALS, INC., Boston, MA (US)

(72) Inventors: Michael John Dey, Sandbach (GB); Jaya Gautam, Boston, MA (US); Barton T. Henderson, Belmont, MA (US); Markus Johnsson, Lund (SE); Stina Lindman, Lund (SE)

(21) Appl. No.: 18/951,301

(22) Filed: Nov. 18, 2024

Related U.S. Application Data
(63) Continuation of application No. 16/764,719, filed on May 15, 2020, now abandoned.
(60) Provisional application No. 62/586,643, filed on Nov. 15, 2017.

(51) Int. Cl. A61K 38/08; A61K 9/00; A61K 47/10; A61K 47/14; A61K 47/18; A61K 47/22

(52) U.S. Cl. CPC A61K 38/08; A61K 9/0019; A61K 47/10; A61K 47/14; A61K 47/183; A61K 47/22

(57) ABSTRACT

This disclosure provides, at least in part, a pharmaceutical product comprising: setmelanotide (also known as RM493); setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or another pharmaceutical composition that has its primary mechanism of action at the MC4 receptor as an agonist (referred to herein as an MC4RA_{P}), e.g., a lipid excipient, and/or a pharmaceutically acceptable carrier. The pharmaceutical product described herein provides a sustained release of setmelanotide or another pharmaceutical composition, which may result in a more desirable pharmacokinetic and pharmacodynamic profile upon administration.

### CLAIM OF PRIORITY

This application is a continuation of U.S. application Ser. No. 16/764,719, filed May 15, 2020, which is a U.S. national phase application and claims the benefit of priority under 35 U.S.C § 371 of International Application No. PCT/US2018/061375, filed Nov. 15, 2018, which claims priority to U.S. Application No. 62/586,643, filed on Nov. 15, 2017, the entire contents of which are incorporated herein by reference.

### BACKGROUND

Many biological compounds are released quickly upon administration in standard formulations. Therefore, there is a need to develop extended release formulations which could extend the duration of compounds in circulation and provide desired pharmacokinetic and pharmacodynamics properties.

### SUMMARY

This disclosure provides, at least in part, a pharmaceutical product comprising: setmelanotide (also known as RM493); setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or another pharmaceutical composition that has its primary mechanism of action at the MC4 receptor as an agonist (referred to herein as an MC4RA_{P}), e.g., BIM-22511 (also known as RM511), BIM-22287, BIM-22512, 001152C, 001543C, 001003C, 001574C, 001555C, 001554C, 001556C, 001358C, 001576C, 001364C, 001258C, or the MC4 receptor agonist comprising SEQ ID NO: 11, as disclosed in WO 2008/147556, hereafter referred to as MC4R-11, as the sole active ingredient, as the sole active ingredient for injection, as the active pharmaceutical ingredient, or as the active pharmaceutical ingredient for injection. The pharmaceutical product comprises lipid excipients, an alcohol, and a polar solvent, or a combination thereof in different molar ratios. The pharmaceutical product can also comprise a pharmaceutically acceptable carrier. The pharmaceutical product described herein provides a sustained release, e.g., a gradual or extended release, e.g., of setmelanotide (or an MC4RA_{P}), which has pharmacological activity, as compared to administration of setmelanotide (or an MC4RA_{P}) alone. The pharmaceutical product also results in a more desirable pharmacokinetic and pharmacodynamic profile upon administration.

In an aspect, the disclosure provides a pharmaceutical product comprising: a) a neutral diacyl lipid and/or a tocopherol; b) a phospholipid; c) an alcohol; d) optionally, a polar solvent, e.g., a buffer, optionally comprising an antioxidant; and e) setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, the sole active ingredient for injection, the active pharmaceutical ingredient, or the active pharmaceutical ingredient for injection.

In another aspect, the disclosure provides a pharmaceutical product comprising: a) a neutral diacyl lipid and/or a tocopherol; b) a phospholipid; c) an alcohol; d) optionally, a polar solvent, e.g., a buffer, optionally comprising an antioxidant; and e) setmelanotide.

In another aspect, the disclosure provides a pharmaceutical product comprising: a) a neutral diacyl lipid and/or a tocopherol; b) a phospholipid; c) an alcohol; d) optionally, a polar solvent, e.g., a buffer, optionally comprising an antioxidant; and e) setmelanotide as the sole active ingredient.

In another aspect, the disclosure provides a pharmaceutical product comprising: a) a neutral diacyl lipid and/or a tocopherol; b) a phospholipid; c) an alcohol; d) optionally, a polar solvent, e.g., a buffer, optionally comprising an antioxidant; and e) setmelanotide as the active pharmaceutical ingredient.

In another aspect, the disclosure provides a pharmaceutical product comprising: a) a neutral diacyl lipid and/or a tocopherol; b) a phospholipid; c) an alcohol; d) optionally, a polar solvent, e.g., a buffer, optionally comprising an antioxidant; and e) setmelanotide as the active pharmaceutical ingredient for injection.

In another aspect, the disclosure provides a pharmaceutical product comprising: a) a neutral diacyl lipid and/or a tocopherol; b) a phospholipid; c) an alcohol; d) optionally, a polar solvent, e.g., a buffer, optionally comprising an antioxidant; and e) an MC4RA_{P}, e.g., BIM-22511, BIM-22287, BIM-22512, 001152C, 001543C, 001003C, 001574C, 001555C, 001554C, 001556C, 001358C, 001576C, 001364C, 001258C, or MC4R-11.

In another aspect, the disclosure provides a pharmaceutical product comprising: a) a neutral diacyl lipid and/or a tocopherol; b) a phospholipid; c) an alcohol; d) optionally, a polar solvent, e.g., a buffer, optionally comprising an antioxidant; and e) an MC4RA_{P}, e.g., BIM-22511, BIM-22287, BIM-22512, 001152C, 001543C, 001003C, 001574C, 001555C, 001554C, 001556C, 001358C, 001576C, 001364C, 001258C, or MC4R-11, as the sole active ingredient.

In another aspect, the disclosure provides a pharmaceutical product comprising: a) a neutral diacyl lipid and/or a tocopherol; b) a phospholipid; c) an alcohol; d) optionally, a polar solvent, e.g., a buffer, optionally comprising an antioxidant; and e) an MC4RA_{P}, e.g., BIM-22511, BIM-22287, BIM-22512, 001152C, 001543C, 001003C, 001574C, 001555C, 001554C, 001556C, 001358C, 001576C, 001364C, 001258C, or MC4R-11, as the sole active ingredient for injection.

In another aspect, the disclosure provides a pharmaceutical product comprising: a) a neutral diacyl lipid and/or a tocopherol; b) a phospholipid; c) an alcohol; d) optionally, a polar solvent, e.g., a buffer, optionally comprising an antioxidant; and e) an MC4RA_{P}, e.g., BIM-22511, BIM-22287, BIM-22512, 001152C, 001543C, 001003C, 001574C, 001555C, 001554C, 001556C, 001358C, 001576C, 001364C, 001258C, or MC4R-11, as the active pharmaceutical ingredient.

In another aspect, the disclosure provides a pharmaceutical product comprising: a) a neutral diacyl lipid and/or a tocopherol; b) a phospholipid; c) an alcohol; d) optionally, a polar solvent, e.g., a buffer, optionally comprising an antioxidant; and e) an MC4RA_{P}, e.g., BIM-22511, BIM-22287, BIM-22512, 001152C, 001543C, 001003C, 001574C, 001555C, 001554C, 001556C, 001358C, 001576C, 001364C, 001258C, or MC4R-11, as the active pharmaceutical ingredient for injection.

In an embodiment, component a) of the composition comprises a neutral diacyl lipid. In embodiment, the neutral diacyl lipid comprises diacyl glycerol. In an embodiment, the neutral diacyl lipid comprises glycerol dioleate (GDO).

In an embodiment, component b) of the composition comprises phosphatidylcholine. In another embodiment, the phospholipid comprises soybean phosphatidylcholine.

In an embodiment, component c) of the composition comprises ethanol. In an embodiment, the ethanol is provided in an amount that is sufficiently great that it provides a solubility of setmelanotide of at least 10 mg/g, 20 mg/g or 30 mg/g.

In an embodiment, component d) of the composition comprises a polar solvent, e.g., a buffer, e.g., a citrate buffer, optionally wherein the pH of the buffer is 6.4. In an embodiment, the polar solvent, e.g., buffer, comprises citrate acid monohydrate. In another embodiment, the polar solvent, e.g., buffer, comprises an additional component, e.g., an antioxidant or a chemical or physical stabilizing agent. In an embodiment, the antioxidant is EDTA. In an embodiment, the polar solvent, e.g., buffer, comprises citric acid monohydrate, disodium EDTA, and water.

In an embodiment, component e) of the composition comprises setmelanotide; setmelanotide as sole active pharmaceutical ingredient formulated for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11). In an embodiment, component e) of the composition comprises setmelanotide.

In an embodiment, component e) provides setmelanotide as the sole active ingredient.

In an embodiment, component e) provides setmelanotide as the active pharmaceutical ingredient.

In an embodiment, component e) provides setmelanotide as the active pharmaceutical ingredient for injection.

In an embodiment, component e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as as the sole active ingredient for injection.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the active pharmaceutical ingredient.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the active pharmaceutical ingredient for injection.

In an embodiment, the composition comprises a neutral diacyl lipid comprising glycerol dioleate; a phospholipid comprising phosphatidylcholine; an alcohol comprising ethanol; a polar solvent, e.g., a buffer comprising a citrate buffer, e.g., at pH 6.4 comprising EDTA; and setmelanotide.

In an embodiment, the composition comprises, per one milliliter of composition, 419.8 mg glycerol dioleate (GDO); 419.8 mg soybean phosphatidylcholine; 105 mg ethanol; 20 mg citrate buffer; and 30 mg setmelanotide.

In another aspect, the disclosure provides an injectable pharmaceutical product comprising: a) a neutral diacyl lipid and/or a tocopherol; b) a phospholipid; c) an alcohol; d) optionally, a polar solvent, e.g., a buffer, optionally comprising an antioxidant; and e) setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11).

In an embodiment component, e) provides setmelanotide as the sole active ingredient.

In an embodiment component, e) provides setmelanotide as the active pharmaceutical ingredient.

In an embodiment component, e) provides setmelanotide as the active pharmaceutical ingredient for injection.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as as the sole active ingredient for injection.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the active pharmaceutical ingredient.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the active pharmaceutical ingredient for injection.

In an embodiment, component a) of the composition comprises a neutral diacyl lipid. In embodiment, the neutral diacyl lipid comprises diacyl glycerol. In an embodiment, the neutral diacyl lipid comprises glycerol dioleate (GDO).

In an embodiment, component b) of the composition comprises phosphatidylcholine. In an embodiment, the phospholipid comprises soybean phosphatidylcholine.

In an embodiment, component c) of the composition comprises ethanol. In an embodiment, the ethanol is provided in an amount that is sufficiently great that it provides a solubility of setmelanotide of at least 10 mg/g, 20 mg/g or 30 mg/g.

In an embodiment, component d) of the composition comprises a polar solvent, e.g., a buffer, e.g., a citrate buffer, optionally wherein the pH of the buffer is 6.4. In an embodiment, the polar solvent, e.g., buffer, comprises citrate acid monohydrate. In an embodiment, the polar solvent, e.g., buffer, comprises an additional component, e.g., an antioxidant or a chemical or physical stabilizing agent. In an embodiment, the antioxidant is EDTA. In an embodiment, the polar solvent, e.g., buffer, comprises citric acid monohydrate, disodium EDTA, and water.

In an embodiment, component e) is setmelanotide. In an embodiment, component e) is an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11).

In an embodiment component, e) provides setmelanotide as the sole active ingredient.

In an embodiment component, e) provides setmelanotide as the active pharmaceutical ingredient.

In an embodiment component, e) provides setmelanotide as the active pharmaceutical ingredient for injection.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as as the sole active ingredient for injection.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the active pharmaceutical ingredient.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the active pharmaceutical ingredient for injection.

In an embodiment, the injectable pharmaceutical product comprises setmelanotide as the active pharmaceutical ingredient. In an embodiment, the injectable pharmaceutical product comprises a neutral diacyl lipid comprising glycerol dioleate; a phospholipid comprising phosphatidylcholine; an alcohol comprising ethanol; a polar solvent, e.g., a buffer comprising a citrate buffer, e.g., at pH 6.4 comprising EDTA; and setmelanotide.

In an embodiment, the injectable pharmaceutical product comprises, per one milliliter of composition, 419.8 mg glycerol dioleate (GDO); 419.8 mg soybean phosphatidylcholine; 105 mg ethanol; 20 mg citrate buffer; and 30 mg setmelanotide.

In another aspect, the disclosure provides a method of making a preparation or composition, e.g., a pharmaceutical composition, comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient, as the sole active ingredient for injection, as the active pharmaceutical ingredient, or as the active pharmaceutical ingredient for injection, e.g., a preparation or composition having a controlled level of EtOH, comprising:
i) providing a mixture of setmelanotide; setmelanotide as sole active pharmaceutical ingredient formulated for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), a first amount of EtOH and one or more of components:
   a) a neutral diacyl lipid and/or a tocopherol;
   b) a phospholipid;
   c) an alcohol; and
   d) a polar solvent, e.g., a buffer; and
ii) adding a second amount of EtOH,
thereby making a preparation or composition, e.g., a pharmaceutical composition, comprising a setmelanotide; setmelanotide as sole active pharmaceutical ingredient formulated for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11).

In an embodiment, the pharmaceutical product comprises setmelanotide.

In an embodiment, the method further comprises making a mixture comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection; as the active pharmaceutical ingredient, or as the active pharmaceutical ingredient for injection; EtOH, a neutral diacyl lipid and/or a tocopherol and a phospholipid,
wherein one or both of the neutral diacyl lipid and/or a tocopherol and phospholipid are added after setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; and the EtOH are combined,
thereby making a setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection composition or preparation, e.g., a pharmaceutical product.

In another aspect, a method of making a pharmaceutical product comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; component a, component b, component d, and a predetermined amount of alcohol, comprises:
combining (in any order) setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; component a, component b, component d and alcohol, to provide a mixture, and
comparing a value for alcohol content in the mixture with a reference value for alcohol content,
thereby making a formulation of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a, component b, component d, and a predetermined amount of alcohol.

In an embodiment, the pharmaceutical product comprises setmelanotide.

In an embodiment, the method further comprises, responsive to the value or comparison, increasing or decreasing the amount of alcohol in the mixture to provide a formulation having a predetermined amount of alcohol. In an embodiment, the method comprises adding an addition amount of alcohol to the mixture.

In another aspect, a method of making a pharmaceutical product comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a (e.g., GDO), component b (e.g., soybean PC), component d (e.g., a polar solvent, e.g., a buffer, e.g., a citrate buffer at pH 6.4), and a predetermined amount of alcohol, comprises:
combining (in any order) setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; component a (e.g., GDO), component b (e.g., soybean PC), component d (e.g., a polar solvent, e.g., a buffer, e.g., a citrate buffer at pH 6.4) and alcohol, to provide a mixture, and
comparing a value for alcohol content in the mixture with a reference value for alcohol content,
thereby making a pharmaceutical product comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a (e.g., GDO), component b (e.g., soybean PC), component d (e.g., a polar solvent, e.g., a buffer, e.g., a citrate buffer at pH 6.4), and a predetermined amount of alcohol.

In an embodiment, the pharmaceutical product comprises setmelanotide.

In an embodiment, the method further comprises adding an addition amount of alcohol to the mixture. In an embodiment, the addition amount of alcohol is greater than the predetermined amount of alcohol.

In an aspect, a method of making a pharmaceutical product comprising component e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, comprises:
(i) providing a mixture comprising component e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11)), as the sole active ingredient, as the sole active ingredient for injection, as the active pharmaceutical ingredient, or as the active pharmaceutical ingredient for injection, and alcohol (a component e)-alcohol mixture), e.g., setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; in contact with, e.g., dissolved or dispersed in an alcohol, e.g., ethanol; and
(ii) combining the component e) -alcohol mixture with an amount of component a, e.g., GDO, component b, e.g., soybean PC, and component d, e.g., citrate buffer at pH 6.4, or all of components a, b, and d.

In an embodiment, the method further comprises performing step (i), (ii), or (i) and (ii) in a closed vessel.

In an aspect, the disclosure provides a method of making a preparation of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, or evaluating a candidate preparation, e.g., for a quality control or release specification, comprising:
providing a value for the amount of EtOH in a candidate preparation of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; and
comparing the value with a reference value for amount of EtOH;
thereby making a preparation of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.

In an embodiment, the pharmaceutical product comprises setmelanotide.

In an aspect, provided herein is a method of making a setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, composition or preparation, e.g., a pharmaceutical product, comprising:
making a mixture comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; EtOH, a neutral diacyl lipid and/or a tocopherol and a phospholipid,
wherein one or both of the neutral diacyl lipid and/or a tocopherol and phospholipid are added after setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, and the EtOH are combined,
thereby making a setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection composition or preparation, e.g., a pharmaceutical product.

In an embodiment, one or both of the neutral diacyl lipid and/or a tocopherol and a phospholipid are added after at least 10%, 25%, 50%, 75%, or all of the setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is allowed to go into solution.

In an embodiment, the order of formation of the mixture is:
i) setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; is contacted with EtOH (and optionally water or buffer);
ii) phospholipid is added to the mixture resulting from i); and
iii) neutral diacyl lipid and/or a tocopherol is added to the mixture resulting from ii).

In an embodiment, the pharmaceutical product comprises setmelanotide.

In another aspect, a method of making a pharmaceutical product of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, comprises the following steps in order:
(i) providing a mixture comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection and alcohol (a component (e)-alcohol mixture), e.g., setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, in contact with, e.g., dissolved or dispersed in, an alcohol, e.g., ethanol; and
(ii) combining the component (e)-alcohol mixture with an amount of component a (e.g., GDO), component b (e.g., soybean PC), and component d (e.g., citrate buffer at pH 6.4), or an amount of all of components a, b, and d;
thereby making a a pharmaceutical product of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, e.g., a pharmaceutical product setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, comprising a predetermined amount of alcohol, e.g., 10 wt. % alcohol, e.g., ethanol.

In an embodiment, the pharmaceutical product comprises setmelanotide.

In an aspect, a method of making a pharmaceutical product of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, comprises the following steps in order:
(i) providing a mixture comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BM-22287. BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; alcohol and component d, e.g., a polar solvent, e.g., a buffer, e.g., a citrate buffer at pH 6.4 (a component (e)-alcohol-buffer mixture), e.g., setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, in contact with, e.g., dissolved or dispersed in, an alcohol, e.g., ethanol, and a citrate buffer at pH 6.4; and
(ii) combining the component (e)-alcohol-buffer mixture with an amount of component a (e.g., GDO), and component b (e.g., soybean PC), or an amount of all of components a and b;
thereby making a pharmaceutical product of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., B1M-22511, BIM-22287, BIM-212512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, e.g., a pharmaceutical product of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C. 001543C, 001003C, 001574C, 001555C. 001554C, 001556C, 001358C, 001576C, 001364C, 001258C, or MC4R-11), as the sole active ingredient, as the sole active ingredient for injection; for injection, as the active pharmaceutical ingredient, or as the active pharmaceutical ingredient for injection, comprising a predetermined amount of alcohol, e.g., 10 wt. % alcohol, e.g., ethanol.

In an embodiment, the pharmaceutical product comprises setmelanotide.

In yet another aspect, a method of making a pharmaceutical product comprising setmelanotide; component a, component b, component d, and a predetermined amount of alcohol, comprises
combining, in a specified order, setmelanotide, component a, component b, component d and an addition amount of alcohol, wherein the addition amount of alcohol results in a predetermined amount of alcohol in the pharmaceutical product, and wherein the specified order comprises the following steps in order:
(i) providing a mixture comprising setmelanotide and an addition amount of alcohol (a setmelanotide-alcohol mixture), e.g., setmelanotide in contact with, e.g., dissolved or dispersed in, an alcohol, e.g., ethanol; and
(ii) combining the setmelanotide-alcohol mixture with an amount of components a, b, and d or all of components a, b, and d;
wherein (i), (ii) or (i) and (ii) are performed in a closed vessel, thereby making a pharmaceutical product comprising setmelanotide, component a, component b, component d, and a predetermined amount of alcohol, e.g., 10 wt. % alcohol, e.g., ethanol.

In another aspect, a method of making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a (e.g., GDO), component b (e.g., soybean PC), component d (e.g., a polar solvent, e.g., a buffer, e.g., a citrate buffer at pH 6.4), and a predetermined amount of alcohol, comprises
combining, in a specified order, component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; component a (e.g., GDO), component b (e.g., soybean PC), component d (e.g., a polar solvent, e.g., a buffer, e.g., a citrate buffer at pH 6.4), and an addition amount of alcohol, wherein the addition amount of alcohol results in a predetermined amount of alcohol in the pharmaceutical product, wherein the specified order comprises the following steps in order:
(i) providing a mixture comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, and an addition amount of alcohol (a component (e)-alcohol mixture), e.g., setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511. BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, in contact with, e.g., dissolved or dispersed in, an alcohol, e.g., ethanol; and
(ii) combining the component (e)-alcohol mixture with an amount of component a (e.g., GDO), component b (e.g., soybean PC), and component d (e.g., citrate buffer at pH 6.4), or all of components a, b, and d;
wherein (i), (ii) or (i) and (ii) are performed in a closed vessel, thereby making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287. BIM-22512, 001152C, 001543C, 001003C, 001574C, 001555C. 001554C. 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; component a (e.g., GDO), component b (e.g., soybean PC), component d (e.g., a polar solvent, e.g., a buffer, e.g., a citrate buffer at pH 6.4), and a predetermined amount of alcohol, e.g., 10 wt. % alcohol, e.g., ethanol.

In an embodiment, the pharmaceutical product comprises setmelanotide.

In another aspect, a method of making a pharmaceutical product comprising setmelanotide, component a (e.g., GDO), component b (e.g., soybean PC), component d (e.g., a polar solvent, e.g., a buffer, e.g., a citrate buffer at pH 6.4), and a predetermined amount of alcohol, comprises
combining, in a specified order, setmelanotide; component a (e.g., GDO), component b (e.g., soybean PC), component d (e.g., a polar solvent, e.g., a buffer, e.g., a citrate buffer at pH 6.4), and an addition amount of alcohol, wherein the addition amount of alcohol results in a predetermined amount of alcohol in the pharmaceutical product, wherein the specified order comprises the following steps in order:
(i) providing a mixture comprising setmelanotide and an addition amount of alcohol (a setmelanotide moiety-alcohol mixture), e.g., setmelanotide in contact with, e.g., dissolved or dispersed in, an alcohol, e.g., ethanol; and
(ii) combining the setmelanotide-alcohol mixture with an amount of component a (e.g., GDO), component b (e.g., soybean PC), and component d (e.g., citrate buffer at pH 6.4), or all of components a, b, and d;
wherein (i), (ii) or (i) and (ii) are performed in a closed vessel, thereby making a pharmaceutical product comprising setmelanotide, component a (e.g., GDO), component b (e.g., soybean PC), component d (e.g., a polar solvent, e.g., a buffer, e.g., a citrate buffer at pH 6.4), and a predetermined amount of alcohol, e.g., 10 wt. % alcohol, e.g., ethanol.

In another aspect, a method of making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, B1M-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a, component b, component d, and a predetermined amount of alcohol, comprises
combining, in a specified order, component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; component a, component b, component d and an addition amount of alcohol, wherein the addition amount of alcohol results in a predetermined amount of alcohol in the pharmaceutical product, wherein the specified order comprises the following steps in order:
(i) providing a mixture comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; an addition amount of alcohol and component d, e.g., a polar solvent, e.g., a buffer, e.g., a citrate buffer at pH 6.4 (a component (e)-buffer mixture), e.g., setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, in contact with, e.g., dissolved or dispersed in, an alcohol, e.g., ethanol and a citrate buffer at pH 6.4; and
(ii) combining the component (e)-alcohol-buffer mixture with an amount of components a and b or all of components a and b;
wherein (i), (ii) or (i) and (ii) are performed in a closed vessel, thereby making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; component a, component b, component d, and a predetermined amount of alcohol, e.g., 10 wt. % alcohol, e.g., ethanol.

In an embodiment, the pharmaceutical product comprises setmelanotide.

In an embodiment component, e) provides setmelanotide as the sole active ingredient.

In an embodiment component, e) provides setmelanotide as the active pharmaceutical ingredient.

In an embodiment component, e) provides setmelanotide as the active pharmaceutical ingredient for injection.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as as the sole active ingredient for injection.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the active pharmaceutical ingredient.

In an embodiment component, e) provides an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the active pharmaceutical ingredient for injection.

In an aspect, a method of making a pharmaceutical product comprising setmelanotide, component a, e.g., GDO, component b, e.g., soybean PC, component d, e.g., citrate buffer at pH 6.4, and a predetermined amount of alcohol, comprises:
combining, in a specified order, the setmelanotide, component a, component b, component d and an addition amount of alcohol, wherein the addition amount of alcohol results in a predetermined amount of alcohol in the pharmaceutical product, wherein the specified order comprises the following steps in order:
(i) providing a mixture comprising a setmelanotide, an addition amount of alcohol and component d, e.g., a polar solvent, e.g., a buffer, e.g., a citrate buffer at pH 6.4 (a setmelanotide-alcohol-buffer mixture), e.g., setmelanotide in contact with, e.g., dissolved or dispersed in, an alcohol, e.g., ethanol and a citrate buffer at pH 6.4; and
(ii) combining setmelanotide-alcohol-buffer mixture with an amount of components a and b or all of components a and b;
wherein (i), (ii) or (i) and (ii) are performed in a closed vessel, thereby making a pharmaceutical product comprising setmelanotide, component a, component b, component d, and a predetermined amount of alcohol, e.g., 10 wt. % alcohol, e.g., ethanol.

In another aspect, a method of making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C. 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; component a (e.g., GDO), component b (e.g., soybean PC), component d (e.g., a polar solvent, e.g., a buffer, e.g., a citrate buffer at pH 6.4), and a predetermined amount of alcohol, comprises
combining, in a specified order, component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; component a, component b, component d and an addition amount of alcohol, wherein the addition amount of alcohol results in a predetermined amount of alcohol in the pharmaceutical product, wherein the specified order comprises the following steps in order:
(i) providing a mixture comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4R Ap (e.g., B1M-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; an addition amount of alcohol and component d, e.g., a polar solvent, e.g., a buffer, e.g., a citrate buffer at pH 6.4 (a component (e)-alcohol-buffer mixture), e.g., component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287. BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; in contact with, e.g., dissolved or dispersed in, an alcohol, e.g., ethanol and a citrate buffer at pH 6.4; and
(ii) combining the component (e)-alcohol-buffer mixture with an amount of component a (e.g., GDO), and component b (e.g., soybean PC) or all of components a and b;
wherein (i), (ii) or (i) and (ii) are performed in a closed vessel, thereby making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; component a (e.g., GDO), component b (e.g., soybean PC), component d (e.g., a polar solvent, e.g., a buffer, e.g., a citrate buffer at pH 6.4), and a predetermined amount of alcohol, e.g., 10 wt. % alcohol, e.g., ethanol.

In an embodiment, the pharmaceutical product comprises setmelanotide.

In an aspect, a method of making a pharmaceutical product comprising setmelanotide, component a (e.g., GDO), component b (e.g., soybean PC), component d (e.g., a polar solvent, e.g., a buffer, e.g., a citrate buffer at pH 6.4), and a predetermined amount of alcohol, comprises:
combining, in a specified order, the setmelanotide, component a, component b, component d and an addition amount of alcohol, wherein the addition amount of alcohol results in a predetermined amount of alcohol in the pharmaceutical product, wherein the specified order comprises the following steps in order:
(i) providing a mixture comprising the setmelanotide, an addition amount of alcohol and component d, e.g., a polar solvent, e.g., a buffer, e.g., a citrate buffer at pH 6.4 (a setmelanotide-alcohol-buffer mixture), e.g., setmelanotide in contact with, e.g., dissolved or dispersed in, an alcohol, e.g., ethanol and a citrate buffer at pH 6.4; and
(ii) combining the setmelanotide-alcohol-buffer mixture with an amount of component a (e.g., GDO), and component b (e.g., soybean PC) or all of components a and b;
wherein (i), (ii) or (i) and (ii) are performed in a closed vessel, thereby making a pharmaceutical product comprising setmelanotide, component a (e.g., GDO), component b (e.g., soybean PC), component d (e.g., a polar solvent, e.g., a buffer, e.g., a citrate buffer at pH 6.4), and a predetermined amount of alcohol, e.g., 10 wt. % alcohol, e.g., ethanol.

In another aspect, a method of making a pharmaceutical product comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, comprises the following steps in order:
i) combining an amount of one or more of (e.g., all of) components a, b, c and d;
ii) providing to this mixture setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, B1M-22287, BIM-22512, 001152C, 001543C, 001003C, 001574C, 001555C, 001554C, 001556C, 001358C. 001576C. 001364C. 001258C. or MC4R-11), as the sole active ingredient, as the sole active ingredient for injection, as the active pharmaceutical ingredient, or as the active pharmaceutical ingredient for injection; and
iii) mixing the mixture of (i) and (ii) for a specified amount of time to dissolve or disperse setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, 131M-22287, B1M-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection,
thereby making a pharmaceutical product setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, 31M-22287, BIM-22512, 001152C, 001543C, 001003C, 001574C, 001555C, 001554C, 001556C, 001358C, 001576C, 001364C, 001258C, or MC4-11), e.g., a pharmaceutical product comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511. BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection; for injection, as the active pharmaceutical ingredient, or as the active pharmaceutical ingredient for injection, comprising a predetermined amount of alcohol, e.g., 10 wt. % alcohol, e.g., ethanol.

In an embodiment, the pharmaceutical product comprises setmelanotide. In an embodiment, the mixing is performed for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours. In an embodiment, the mixing is performed for 1-30 hours. In another embodiment, the mixing is performed for no more than 30, 40, or 50 hours.

In an aspect, a method of making a pharmaceutical product comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, comprises the following steps in order:
i) combining an amount of one or more of (e.g., all of) components a, b, d and setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, 1M-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection;
ii) providing to this mixture a predetermined amount of component c; and
iii) mixing the mixture of (i) and (ii) for a specified amount of time to dissolve or disperse setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, 1M-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection,
thereby making a pharmaceutical product setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287. BIM-22512, 001152C. 001543C, 001003C, 001574C, 001555C. 001554C. 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, e.g., a pharmaceutical product comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, comprising a predetermined amount of alcohol, e.g., 10 wt. % alcohol, e.g., ethanol.

In an embodiment, the pharmaceutical product comprises setmelanotide.

In an embodiment, the mixing is performed for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours. In an embodiment, the mixing is performed for 1-30 hours. In another embodiment, the mixing is performed for no more than 30, 40, or 50 hours.

In another aspect, disclosed herein is a method of providing setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, to a subject, comprising administering to the subject, and effective amount of a pharmaceutical product described herein, or a pharmaceutical product made by one or more of the methods described herein.

In an embodiment, the subject has, or is at risk of having a disorder responsive to modulation of melanocortin-4 receptor (MC4R). In an embodiment, the disorder is chosen from: type 1 diabetes, type 2 diabetes, obesity, insulin resistance, metabolic syndrome, male erectile dysfunction, female sexual disorder, non-alcoholic fatty liver disease, non-alcoholic steatohepatitis, disorders of substance abuse, including alcoholism, feeding disorders, cachexia, inflammation or anxiety. In an embodiment, the disorder is obesity. In an embodiment, the disorder is type 1 diabetes. In an embodiment, the disorder is type 2 diabetes. In an embodiment, the disorder is Prader-Willi Syndrome. In an embodiment, the disorder is Bardet-Biedl syndrome. In an embodiment, the disorder is Alström syndrome.

In an embodiment, the subject is between 1-80 years of age. In an embodiment, the subject is between 1-10 years old, between 10-20 years old, between 20-30 years old, between 30-40 years old, between 40-50 years old, between 50-60 years old, between 60-70 years old or between 70-80 years old. In an embodiment, the subject is more than 80 years old.

In a related aspect, the disclosure provides a method of treating a subject comprising administering to a subject in need thereof, an effective amount of a composition comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511. BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.

In an embodiment, the subject has, or is at risk of having a disorder responsive to modulation of melanocortin-4 receptor (MC4R). In an embodiment, the disorder is chosen from: type 1 diabetes, type 2 diabetes, obesity, insulin resistance, metabolic syndrome, male erectile dysfunction, female sexual disorder, non-alcoholic fatty liver disease, non-alcoholic steatohepatitis, disorders of substance abuse, including alcoholism, feeding disorders, cachexia, inflammation or anxiety. In an embodiment, the disorder is obesity. In an embodiment, the disorder is type 1 diabetes. In an embodiment, the disorder is type 2 diabetes. In an embodiment, the disorder is Prader-Willi Syndrome. In an embodiment, the disorder is Bardet-Biedl syndrome. In an embodiment, the disorder is Alström syndrome.

In an embodiment, the subject is between 1-80 years of age. In an embodiment, the subject is between 1-10 years old, between 10-20 years old, between 20-30 years old, between 30-40 years old, between 40-50 years old, between 50-60 years old, between 60-70 years old or between 70-80 years old. In an embodiment, the subject is more than 80 years old.

In an aspect, provided herein is a composition comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, for use in treating a subject in need thereof, comprising administering to the subject an effective amount of the composition.

In an embodiment, the subject has, or is at risk of having a disorder responsive to modulation of melanocortin-4 receptor (MC4R). In an embodiment, the disorder is chosen from: type 1 diabetes, type 2 diabetes, obesity, insulin resistance, metabolic syndrome, male erectile dysfunction, female sexual disorder, non-alcoholic fatty liver disease, non-alcoholic steatohepatitis, disorders of substance abuse, including alcoholism, feeding disorders, cachexia, inflammation or anxiety. In an embodiment, the disorder is obesity. In an embodiment, the disorder is type 1 diabetes. In an embodiment, the disorder is type 2 diabetes. In an embodiment, the disorder is Prader-Willi Syndrome. In an embodiment, the disorder is Bardet-Biedl syndrome. In an embodiment, the disorder is Alström syndrome.

In an embodiment, the subject is between 1-80 years of age. In an embodiment, the subject is between 1-10 years old, between 10-20 years old, between 20-30 years old, between 30-40 years old, between 40-50 years old, between 50-60 years old, between 60-70 years old or between 70-80 years old. In an embodiment, the subject is more than 80 years old.

Optimized pharmaceutical products comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}, e.g., BIM-22511, BIM-22287, BIM-22512, 001152C, 001543C, 001003C, 001574C, 001555C, 001554C, 001556C, 001358C, 001576C, 001364C, 001258C, or MC4R-11, as the sole active ingredient, as the sole active ingredient for injection, as the active pharmaceutical ingredient, or as the active pharmaceutical ingredient for injection, and methods of making and using the same, are provided herein. Typically the pharmaceutical product comprises a neutral diacyl glycerol and/or a tocopherol, a phosphatidyl choline, an alcohol, a polar solvent (optionally comprising an antioxidant), and setmelanotide (or an MC4RA_{P} disclosed herein). In an embodiment, the pharmaceutical product has a low viscosity phase, such as an L₂ (reversed micellar) phase. Pharmaceutical products disclosed herein provide a controlled release of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.

In an embodiment, the pharmaceutical product provides an optimized release profile, is relatively easy to manufacture, can be sterile-filtered, has low viscosity upon delivery (allowing easy and less painful administration through, e.g., a narrow needle, e.g., a 27 Gauge or smaller diameter needle), allows a high level of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, to be incorporated (thus in embodiments, allowing a smaller amount of composition and/or setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection to be used), allows shallow injection (e.g., injection into the subcutaneous tissue layer) and/or forms a depot, e.g., a non-lamellar depot, composition in vivo, e.g., a depot having a “non-burst” release profile. In an embodiment, the pharmaceutical product is formed from materials that are non-toxic, biotolerable and biodegradable, which can be administered by intra-muscular (i.m.), or subcutaneous (s.c.) administration, and are suitable for self-administration. In an embodiment, the pharmaceutical product minimizes or eliminates irritation on injection (including transient irritation).

While not wishing to be bound by theory, it is believed, that in an embodiment, the pharmaceutical product generates a crystalline phase, e.g., a non-lamellar liquid crystalline phase, after administration.

In an embodiment, a pharmaceutical product provides low initial release (“non-burst profile”) of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11, as the sole active ingredient, as the sole active ingredient for injection, as the active pharmaceutical ingredient, or as the active pharmaceutical ingredient for injection). This may be defined such that the partial area under the drug concentration curve during the first hours (e.g., first 6, 9, 12, 15, 18, 21, or 24 hours) after dosing, is less than 40% (e.g., less than 35, 30, 25, 20, 15, 10% or lesser), relative to the area under the drug concentration time curve of a 7 day (e.g., 1, 2, 3, 4, 5, 6, 7, 8, 9, 10, 14, 21, or 28 days) steady state dosing interval. In an embodiment, the partial area under the drug concentration curve during the first 6 hours after dosing is less than 10% relative to the area under the drug concentration time curve of a 7 day (e.g., 168 hour) steady state dosing interval. In an embodiment, the partial area under the drug concentration curve during the first 12 hours after dosing is 10-20% (e.g., 10, 11, 12, 13, 14, 15, 16, 17, 18, 19, or 20%), relative to the area under the drug concentration time curve of a 7 day (e.g., 168 hour) steady state dosing interval. In an embodiment, the partial area under the drug concentration curve during the first 24 hours after dosing is 20-30% (e.g., 20, 21, 22, 23, 24, 25, 26, 27, 28, 29, or 30%), relative to the area under the drug concentration time curve of a 7 day (e.g., 168 hour) steady state dosing interval.

In an embodiment, the pharmaceutical product provides a mean maximum drug concentration (Cₘₐₓ) in plasma at steady state in the range of 5-15 ng/mL (e.g., 11 ng/mL) following an injected dose in the range of 5-20 mg (e.g., an injected dose of 10 mg) once weekly (e.g., once every 4, 5, 6, 7, 8, 9, 10, 11, 12, 13, or 14 days), which is no more than 3 times higher (e.g., 1, 1.5, 2, or 2.5 times higher) than the average drug concentration (Cₐᵥₑ) in the range of 1-8 ng/mL (e.g., 4 ng/mL). In an embodiment, the pharmaceutical product provides a mean maximum drug concentration (Cₘₐₓ) in plasma at steady state of 11 ng/ml, following an injected dose of 10 mg once weekly, which is no more than 3 times higher than the average drug concentration (Cₐᵥₑ) of 4 ng/mL.

In an embodiment, the minimum drug concentration (Cₘᵢₙ) in plasma at steady state is in the range of 1-3 ng/mL (e.g., 1.88 ng/mL) at the time of dosing. In an embodiment, the mean accumulation index is in the range of 1-3 (e.g., 1.48), and the mean fluctuation factor is in the range of 180-210% (e.g., 199%). In an embodiment, the setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, profile in plasma at steady state provides drug coverage over a one-week (e.g., a 4, 5, 6, 7, 8, 9, 10, 11, 12, 13, or 14 day) dosing interval.

In an embodiment, the setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, described herein, is optimized to allow for the neutral diacyl lipid and/or tocopherol and phospholipid components to be formulated in a wide range of proportions. In an embodiment, the pharmaceutical product has a greater proportion of neutral diacyl lipid and/or tocopherol to phospholipid than was previously achievable without risking phase separation and/or unacceptably high viscosities in the formulation. In an embodiment the ratio of neutral diacyl lipid and/or tocopherol to phospholipid is 1:1, or a ratio that is between 0.5 to 1.0, 0.7 to 1.0, or 1 to 0.5, or 1 to 0.7.

Methods described herein allow for more precise and accurate levels of alcohol in the preparations. The level of alcohol may affect release kinetics, e.g., burst profile. Levels that are too high may impact, e.g., negatively impact, release kinetics, e.g., burst profile. Control of the level of alcohol thus may allow for a more predictable and repeatable achievement of an optimized release profile. The level of alcohol may also affect pre-injection viscosity, with higher levels of alcohol resulting in lower viscosity. Thus, defined and repeatable levels of alcohol may allow for optimized performance. Described herein are methods that combine the addition of a known amount of alcohol with steps to minimize or control the level of loss by evaporation. In an embodiment, a method of making a pharmaceutical product described herein comprises the use of a controlled amount of alcohol to make the pharmaceutical product. In an embodiment, the pharmaceutical product is prepared so as to reduce or control evaporation, e.g., by the use of a closed vessel (thus reducing or controlling evaporation). The use of a closed vessel may allow for tight regulation of the amount of alcohol that is used to make the pharmaceutical product, and may improve the reproducibility of the pharmaceutical product. Preventing loss from evaporation is an important feature to reduce variability between batches of the pharmaceutical product, and between individual dosage units within a batch. Since the formulation may be used to deliver setmelanotide; setmelanotide as sole active pharmaceutical ingredient formulated for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection in vivo, it is important that each batch of the pharmaceutical product contain a similar amount of the setmelanotide; setmelanotide as sole active pharmaceutical ingredient formulated for injection; or an MC4RA_{P}, at a similar concentration.

Disclosed herein, inter alia, is a specified order in which components are added to the preparation. The specified order may have a significant effect on the solubility of the components with one another, e.g., the solubility of the setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient, as the sole active ingredient for injection, as the active pharmaceutical ingredient, or as the active pharmaceutical ingredient for injection in the preparation. In an embodiment, a method of making the pharmaceutical product described herein comprises a specified order of addition of components to make the pharmaceutical product. In an embodiment, setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection is contacted with one or more of the other components after the setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection is contacted with alcohol. The method may further optimize the time required for making the preparation. The pharmaceutical product of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection resulting from a specific order of addition of components has a low viscosity and an amount of alcohol that is compatible with in vivo use.

In embodiments, the control of alcohol content and the order of addition can be combined.

The disclosure contemplates all combinations of any one or more of the foregoing aspects and/or embodiments, as well as combinations with any one or more of the embodiments set forth in the detailed description and examples.

Although methods and materials similar or equivalent to those described herein can be used in the practice or testing of the present invention, suitable methods and materials are described below. All publications, patent applications, patents, and other references (e.g., sequence database reference numbers) mentioned herein are incorporated by reference in their entirety.

### BRIEF DESCRIPTION OF THE DRAWINGS

FIGS. 1A-B are graphs depicting concentration time profiles from subcutaneous injections of setmelanotide formulations 1A-1C (the components of these formulations are provided in Table 4), 5A-5C (the components of these formulations are provided in Table 2), and 3A/4A (the components of these formulations are provided in Table 3); and from an SC infusion of setmelanotide. FIG. 1A depicts the concentration data in a linear scale and, FIG. 1B depicts the concentration data in a semi-logarithmic scale.

FIGS. 2A-B are graphs depicting concentration time profiles from subcutaneous injections of setmelanotide formulations 1D-1E (the components of these formulations are provided in Table 4), and 5D (the components of this formulations is provided in Table 2); and from an SC infusion of setmelanotide. FIG. 2A depicts the concentration data in a linear scale and, FIG. 2B depicts the concentration data in a semi-logarithmic scale.

FIG. 3 is a graph showing the mean setmelanotide concentration (ng/mL) in plasma after a single subcutaneous dose (30 mg, 10 mg, or 2.5 mg) was administered to healthy subjects.

FIG. 4 is a graph depicting the trough concentrations of setmelanotide (ng/mL) in plasma for subjects after multiple dosing of a subcutaneous injection of a long-acting setmelanotide formulation every week for four weeks.

FIG. 5 is a graph showing the percent degradation of setmelanotide in an exemplary formulation, wherein the GDO component was either i) GDO of commercial purity; ii) GDO of commercial purity, further purified to remove fatty acid compounds; iii) or synthetic GDO.

FIG. 6 is a graph depicting the effect of EDTA concentration on stability of an exemplary setmelanotide formulation.

FIG. 7 is a graph depicting the effect of setmelanotide concentration in an exemplary setmelanotide formulation and its effect on degradation.

### DETAILED DESCRIPTION

This disclosure provides, at least in part, a pharmaceutical product comprising: setmelanotide (also known as RM493); setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or another pharmaceutical composition that has its primary mechanism of action at the MC4 receptor as an agonist (referred to as an MC4RA_{P}), e.g., BIM-22511 (also known as RM511), BIM-22287, BIM-22512, 001152C, 001543C, 001003C, 001574C, 001555C, 001554C, 001556C, 001358C, 001576C, 001364C, 001258C, or the MC4 receptor agonist disclosed as SEQ ID NO: 11 in WO 2008/147556 (referred to as MC4R-11), as the sole active ingredient, as the sole active ingredient for injection, as the active pharmaceutical ingredient, or as the active pharmaceutical ingredient for injection.

The pharmaceutical product of the disclosure generates a non-lamellar liquid crystalline phase following administration. The use of non-lamellar phase structures (such as liquid crystalline phases) in the delivery of bioactive agents is known to the field. Exemplary lipid depot systems are described, e.g., in WO 2005/117830, the entire contents of which are hereby incorporated by reference.

All % are specified by weight herein throughout, unless otherwise indicated. Furthermore, the % by weight indicated is the % of the total pharmaceutical product including all of the components indicated herein. The pharmaceutical product can optionally consist of essentially only the components indicated herein (including where appropriate additional optional components indicated herein below and in the attached claims) and in one aspect consist entirely of such components.

In an embodiment, a pharmaceutical product has an L₂ phase structure. In an embodiment, the pharmaceutical product forms a non-lamellar, e.g., liquid crystalline, phase following administration.

In an embodiment, a pharmaceutical product allows control of the peak concentration (Cₘₐₓ) of drug in the plasma to a level equal to or less than that tolerable to the subject, for example to avoid side-effects such as headache, alteration in blood pressure, heart rate, respiration rate, excessive perspiration, or other unwanted or undesirable effects while providing or achieving, an effective, e.g., therapeutically effective, level over the period of release. Generally, the average concentration during the period of release before the next dose is administered, Cₐᵥₑ, falls within the therapeutic range. Control over the maximal (Cₘₐₓ) and minimum (Cₘᵢₙ) concentrations over the defined dosage period, e.g. once weekly, or once monthly, is also important in order to achieve effective and safe treatment over time. In one embodiment, the initial burst is not the Cₘax of the release profile.

Whether or not the initial burst is also the Cₘₐₓ, the Cₘₐₓ/Cₐᵥₑ ratio is less than or equal to 10. In an embodiment, the Cₐᵥₑ/Cₘᵢₙ ratio is less than or equal to 10, e.g., less than or equal to 9, 8, 7, 6, 5, 4, 3, 2, or 1. In an embodiment, the Cₐᵥₑ/Cₘᵢₙ ratio is less than 3. Cₘₐₓ is defined as the peak or maximal plasma concentration observed during the period of release before the next dose is administered and Cₐᵥₑ is defined as the average plasma concentration during that period of release. Cₘᵢₙ is correspondingly the minimal concentration during that period. Cₐᵥₑ can be calculated by calculating the drug present in the plasma as area under the curve (AUC) over the selected period of time, generally the entire period of release before the administration of the next dose, and dividing by that period of time.

Component (a) Neutral Diacyl Lipid and/or Tocopherol

Component “a” as described herein is a neutral lipid comprising a polar “head” group and non-polar “tail” groups. Generally, the head and tail portions of the lipid will be joined by an ester moiety but this attachment may be by means of an ether, an amide, a carbon-carbon bond or other attachment. In an embodiment, the polar head group comprises a non-ionic head group including polyols, e.g., glycerol, diglycerol or sugar moieties (e.g., inositol or glucosyl based moieties), and esters of polyols, e.g., acetate or succinate esters. In an embodiment, the polar “head” group comprises glycerol or diglycerol.

In an embodiment, component (a) comprises a diacyl lipid with two non-polar “tail” groups. The two non-polar groups may have the same or a differing number of carbon atoms and may each independently be saturated or unsaturated. In embodiments, the two non-polar “tail” groups may have the same or a differing number of carbon atoms and may each independently be saturated or unsaturated. In an embodiment, the non-polar groups include C6-C32 alkyl and alkenyl groups, which are typically present as the esters of long chain carboxylic acids. These are often described by reference to the number of carbon atoms and the number of unsaturations in the carbon chain. Thus, CX:Z indicates a hydrocarbon chain having X carbon atoms and Z unsaturations.

In an embodiment, the non-polar “tail” groups of component (a) are chosen from: lauroyl (C12:0), myristoyl (C14:0), palmitoyl (C16:0), phytanoyl (C16:0), palmitoleoyl (C16:1), stearoyl (C18:0), oleoyl (C18:1), elaidoyl (C18:1), linoleoyl (C18:2), linolenoyl (C18:3), arachidonoyl (C20:4), behenoyl (C22:0) or lignoceroyl (C24:9). Typically, non-polar chains are based on the fatty acids of natural ester lipids, including caproic, caprylic, capric, lauric, myristic, palmitic, phytanic, palmitolic, stearic, oleic, elaidic, linoleic, linolenic, arachidonic, behenic or lignoceric acids, or the corresponding alcohols. In an embodiment, the non-polar “tail” group of component (a) is chosen from palmitic, stearic, oleic or linoleic acid. In an embodiment, the non-polar “tail” group of component (a) is oleic acid.

In an embodiment, the diacyl lipid of component (a) is chosen from: Butyric (C4), Valeric (C5), Caproic (C6), Enanthic (C7), Caprylic (C8), Pelargonic (C9), Capric (C10), Undecylic (C11), Lauric (C12), Tridecylic (C13), Myristic (C14), Pentadecanoic (C15), Palmitic (C16), Margaric (C17), Stearic (C18), Nonadecylic (C19), Arachidic (C20), Heneicosylic (C21), Behenic (C22), Tricosylic (C23), Lignoceric (C24), Pentacosylic (C25), Cerotic (C26), Heptacosylic (C27), Montanic (C28), Nonacosylic (C29), Melissic (C30), Hentriacontylic (C31), Lacceroic (C32,) Psyllic (C33), Geddic (C34), Ceroplastic (C35), Hexatriacontylic (C36), Heptatriacontanoic (C37), Octatriacontanoic (C38), α-Linolenic (18:3), Stearidonic (18:4), Eicosapentaenoic (20:5), Docosahexaenoic (22:6), Linoleic (18:2), γ-Linolenic (18:3), Dihomo-γ-linolenic (20:3), Arachidonic (20:4), Adrenic (22:4), Palmitoleic (16:1), Vaccenic (18:1), Paullinic (20:1), Oleic (18:1), Elaidic (trans-18:1) Gondoic (20:1), Erucic (22:1), Nervonic (24:1), and Mead (20:3), or any pharmaceutically acceptable esters formed with acids.

In an embodiment, mixtures of any number of diacyl lipids may be used as component (a). In an embodiment, component (a) comprises a portion of C18 lipids (e.g., diacyl glycerol having one or more C18:0, C18:1, C18:2 or C18:3 non-polar “tail” groups), e.g., glycerol dioleate (GDO) or glycerol dilinoleate (GDL).

In an embodiment, the diacyl lipid of component (a) comprises a diacyl glycerol (DAG), e.g., glycerol dioleate (GDO). In an embodiment, the DAG of component (a) is GDO.

In an embodiment, component (a) is DAG comprising at least 50 wt. %, at least 80 wt. %, at least 90 wt. %, at least 95 wt. %, or 100 wt. % GDO. In an embodiment, component (a) comprises 100 wt. % GDO.

In an embodiment, the diacyl lipid, when used as all or part of component (a), may be synthetic or may be derived from a purified and/or chemically modified natural source, e.g., vegetable oils. In an embodiment, the diacyl lipid (e.g., the DAG, e.g., GDO), is synthetic. In an embodiment, the diacyl lipid (e.g., the DAG, e.g., GDO) is derived from a natural source, e.g., a plant source or a bacterial source.

In an embodiment, the diacyl lipid has a purity of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.9%, or more. In some embodiment, the diacyl lipid is GDO. In an embodiment, the GDO has a purity of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.9%, or more. In an embodiment, the GDO has a purity of at least 50%. In an embodiment, the GDO has a purity of at least 75%. In an embodiment, the GDO has a purity of at least 85%. In an embodiment, the GDO has a purity of at least 90%. In an embodiment, the GDO has a purity of at least 95%. In an embodiment, the GDO has a purity of at least 98%. In an embodiment, the GDO has a purity of at least 99%. In an embodiment, the GDO has a purity between about 50% to 100%, about 75% to about 100%, or about 90% to 100%. In an embodiment, the GDO has a purity between about 85% to 99%, about 88% to 96%, about 90% to 98%, about 90% to 96%, about 90% to 94%, about 90% to 92%, about 92% to 98%, about 92% to 96%, about 92% to 94%, about 94% to 98%, about 94% to 96%, about 96% to 98%, or 98% to 100%.

In an embodiment, GDO as described herein refers to any commercial grade of GDO with concomitant impurities (i.e. GDO of commercial purity). Exemplary impurities include undesired lipids and fatty acid compounds. These impurities may be separated and removed by purification. In an embodiment, GDO comprises chemically pure GDO, e.g., at least 80% pure, at least 85% pure, at least 90% pure, at least 95% pure, at least 99% pure, or 100% pure GDO. In an embodiment, the GDO contains less than about 50%, 45%, 40%, 35%, 30%, 25%, 20%, 15%, 10%, 5%, 2.5%, 1%, or 0.5% of an impurity, e.g., an undesired lipid or a fatty acid compound. In an embodiment, the GDO contains less than about 20% of an impurity, e.g., an undesired lipid or a fatty acid compound. In an embodiment, the GDO contains less than about 10% of an impurity, e.g., an undesired lipid or a fatty acid compound. In an embodiment, the GDO contains less than about 5% of an impurity, e.g., an undesired lipid or a fatty acid compound. In an embodiment, the GDO contains less than about 2% of an impurity, e.g., an undesired lipid or a fatty acid compound. In an embodiment, the GDO contains less than about 1% of an impurity, e.g., an undesired lipid or a fatty acid compound.

In an embodiment, the undesired lipid or fatty acid compound is a saturated lipid or unsaturated lipid. Exemplary undesired lipids or fatty acid compounds include propionic acid, butyric acid, valeric acid, caproic acid, enanthic acid, caprylic acid, pelargonic acid, capric acid, undecylic acid, tridecylic acid, myristic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid hexatriacontylic acid, heptatriacontanoic acid, octatriacontanoic acid, myristoleic acid, palmitoleic acid, vaccenic acid, cis-vaccenic acid, paullinic acid, oleic acid, elaidic acid, 11-eicosenoic acid (i.e., gondoic acid), erucic acid, brassidic acid, nervonic acid, sapienic acid, gadoleic acid, petroselinic acid, and linoleic acid (e.g., alpha-linolenic acid), or an isomer or variant thereof.

In an embodiment, component (a) comprises a tocopherol. In an embodiment, the tocopherol of component (a) comprises a non-ionic lipid tocopherol, e.g., Vitamin E, or any suitable salts or analogs thereof. In an embodiment, a suitable analog of tocopherol comprises those providing the following characteristics: phase-behavior, lack of toxicity, and phase change upon exposure to aqueous fluids.

In an embodiment, the tocopherol is purified from a natural source, and may comprise less than 10 wt. % of a non-tocopherol “contaminant”, e.g., comprising less than 10, 5, 4, 3, 2, 1 wt. % or lesser of the contaminant. In an embodiment, component (a) comprises at least 50, 80, 85, 90, 95, 99 wt. % or more of a tocopherol, e.g., Vitamin E, or any suitable salts or analogs thereof. In an embodiment, component (a) comprises 100 wt. % tocopherol, e.g., Vitamin E, or any suitable salts or analogs thereof.

### Component (b) —Phosphatidyl Choline

Component “b” as described herein comprises a phospholipid comprising a polar head group and at least one non-polar tail group. The non-polar tail group may be derived from the fatty acids or corresponding alcohols described above for component a. In an embodiment, the phospholipid of component (b) contains two non-polar tail groups. In one embodiment, the phospholipid of component (b) comprises the C18 groups and may be combined with any other suitable non-polar tail group, particularly the C16 groups. In one embodiment, the phospholipid of component (b) comprises two identical non-polar tail groups.

In an embodiment, the phospholipid of component (b) comprises a polar head group chosen from: phosphatidylcholine, phosphatidylethanolamine, phosphatidylserine or phosphatidylinositol. In an embodiment, the polar head group of the phospholipid of component (b) is phosphatidylcholine (PC). In an embodiment, component (b) comprises at least 50 wt. %, at least 70 wt. %, at least 80 wt. %, at least 90 wt. %, at least 95 wt. %, at least 99 wt. % or 100 wt. % PC. In an embodiment, component (b) comprises PC.

In an embodiment, the PC of component (b), may be naturally derived, e.g., egg PC, heart PC (e.g., bovine heart PC), brain PC, liver PC (e.g., bovine brain PC, or bovine liver PC) and plants (e.g., soybean PC). In an embodiment, the naturally derived PC may comprise any mixture of phospholipids. In an embodiment, the naturally derived PC comprises soy PC or egg PC. In an embodiment, the naturally derived PC comprises soy PC. In an embodiment, the PC comprises at least 50 wt. % soy PC or egg PC, at least 75 wt. % soy PC or egg PC, at least 80 wt. % soy PC or egg PC, at least 90 wt. % soy PC or egg PC, at least 95 wt. % soy PC or egg PC, at least 99 wt. % soy PC or egg PC, or 100 wt. % soy PC or egg PC.

In an embodiment, the PC of component (b) is soybean PC. In an embodiment, the PC comprises 18:2 fatty acids as the primary fatty acid component with 16:0 and/or 18:1 as the secondary fatty acid components, wherein the ratio of primary acid component to the secondary acid component is between 1.5:1 and 6:1. In an embodiment, the PC of component (b) comprises 60-65% 18:2, 10 to 20% 16:0, 5-15% 18:1, with the balance predominantly other 16 carbon and 18 carbon fatty acids. In an embodiment, the PC comprising of soybean PC has the aforesaid composition.

In an embodiment, the PC of component (b) comprises synthetic dioleoyl PC. Synthetic dioleoyl PC is believed to provide increased stability and is thus suitable for compositions needing to be stable to long term storage, and/or having a long release period in vivo. In an embodiment, the PC of component (b) comprises at least 50 wt. %, at least 75 wt. %, at least 80 wt. %, at least 90 wt. %, at least 95 wt. %, at least 99 wt. % or 100 wt. % of synthetic dioleoyl PC.

In an embodiment, the phospholipid of component (b) has a purity of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.9%, or more. In some embodiments, the phospholipid of component (b) is phosphatidylcholine (PC). In an embodiment, the PC has a purity of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.9%, or more.

In an embodiment, component (b) comprises synthetic or highly purified PCs, e.g., dioleoyl phosphatidyl choline (DOPC). In an embodiment, the synthetic dioleoyl PC comprises 1,2-dioleoyl-sn-glycero-3-phosphocholine, and other synthetic PC components e.g., DDPC (1,2-Didecanoyl-sn-glycero-3-phosphocholine); DEPC (1,2-Dierucoyl-sn-glycero-3-phosphocholine); DLOPC (1,2-Dilinoleoyl-sn-glycero-3-phosphocholine); DLPC (1,2-Dilauroyl-sn-glycero-3-phosphocholine); DMPC (1,2-Dimyristoyl-sn-glycero-3-phosphocholine); DOPC (1,2-Dioleoyl-sn-glycero-3-phosphocholine); DPPC (1,2-Dipalmitoyl-sn-glycero-3-phosphocholine); DSPC (1,2-Distearoyl-sn-glycero-3-phosphocholine); MPPC (1-Myristoyl-2-palmitoyl-sn-glycero 3-phosphocholine); MSPC (1-Myristoyl-2-stearoyl-sn-glycero-3-phosphocholine); PMPC (1-Palmitoyl-2-myristoyl-sn-glycero-3-phosphocholine); POPC (1-Palmitoyl-2-oleoyl-sn-glycero-3-phosphocholine); PSPC (1-Palmitoyl-2-stearoyl-sn-glycero-3-phosphocholine); SMPC (1-Stearoyl-2-myristoyl-sn-glycero-3-phosphocholine); SOPC (1-Stearoyl-2-oleoyl-sn-glycero-3-phosphocholine); and SPPC (1-Stearoyl-2-palmitoyl-sn-glycero-3-phosphocholine), or any combination thereof.

In embodiments, component (b) comprising synthetic or highly purified PCs (e.g. DOPC) may provide greater stability for the active agent in the formulations. In an embodiment, wherein the formulation does not comprise an antioxidant, component (b) comprises (e.g., at least 75 wt. %) synthetic or highly purified (e.g., purity >90%) PCs (e.g., DOPC). In an embodiment, wherein the formulation comprises an antioxidant, component (b) comprises (e.g., comprises at least 75 wt. %) naturally derived PCs, e.g., soy PC or egg PC.

In an embodiment, the pharmaceutical product comprises component (a) in the ranges of 20-80 wt. %, 30-70 wt. %, 33-60 wt. %, or 38-43 wt. %. In an embodiment, the pharmaceutical product comprises component (b) in the ranges of 20-80 wt. %, 30-70 wt. %, 33-55 wt. %, or 38 to 43 wt. %.

In an embodiment, the pharmaceutical product comprises 42 wt. % of component (a). In an embodiment, the pharmaceutical product comprises 42 wt. % of component (a), wherein the neutral diacyl lipid is glycerol dioleate (GDO).

In an embodiment, the pharmaceutical product comprises 42 wt. % of component (b). In an embodiment the pharmaceutical product comprises 42 wt. % of component (b), wherein the phospholipid is a phosphatidylcholine (PC). In an embodiment, the PC is soybean PC.

In an embodiment, the pharmaceutical product comprises at least 80, 70, 60, 50, 40, 30, or 20 wt. % component (a). In an embodiment the pharmaceutical product comprises at least 80, 70, 60, 50, 40, 30, or 20 wt. % component (b). In an embodiment the pharmaceutical product comprises at least 42% component (a), e.g., GDO, and at least 42% component (b), e.g., soybean PC.

In an embodiment, the pharmaceutical product comprises component (a) and (b), wherein component (a), e.g., GDO, is present at 42 wt. %+/−10, 42 wt. %+1-5, 42 wt. %+/−2, or 42 wt. %+/−1.

In an embodiment, the pharmaceutical product comprises component (a) and (b), wherein component (b), e.g., soybean PC, is present at 42 wt. %+/−10, 42 wt. %+1-5, 42 wt. %+/−2, or 42 wt. %+/−1.

In an embodiment, the pharmaceutical product comprises component (a) and (b), wherein component (a), e.g., GDO, and component (b), e.g., soybean PC, is each present at 42 wt. %+/−10, 42 wt. %+1-5, 42 wt. %+/−2, or 42 wt. %+/−1.

In an embodiment, the ratio of component (a):(b) is 40:60 to 70:30, 45:55 to 55:45 or 40:60 to 54:46. In an embodiment, the ratio of (a):(b) is 50:50.

In an embodiment, components (a) and (b) comprise 95 wt. %, e.g., at least 95, 96, 97, 98, 99 wt. % or more, of the lipid components of the pharmaceutical product. In an embodiment, components (a) and (b) comprise 99 wt. % of the total lipid content of the formulation. In an embodiment, the lipid component of the pharmaceutical product comprises essentially all of, e.g., 100 wt. %, components (a) and (b).

### Component (c) —Alcohol

Component (c) as described herein comprises an alcohol. Since the pharmaceutical product is useful to generate a depot composition following administration (e.g., in vivo), typically upon contact with excess aqueous fluid, it is desirable that the alcohol be tolerable to the subject and be capable of mixing with the aqueous fluid, and/or diffusing or dissolving out of the formulation into the aqueous fluid. In an embodiment, component (c) comprises alcohols having at least moderate water solubility.

In an embodiment, the alcohol of component (c) comprises ethanol, propanol, isopropanol, or mixtures thereof. In an embodiment, component (c) comprises ethanol.

In an embodiment, addition or presence of less than 20 wt. % of the alcohol (e.g., less than 20, 15, 14, 13, 12, 11, 10, 9, 8, 7, 6, 5, 4, 3.5, 3, 2.5, 2, 1 wt. % or lesser) to a mixture comprising (a) and (b) gives large viscosity reductions, e.g., viscosity reductions of 30-70%, e.g., 30, 40, 50, 60 or 70% reduction, or more. In an embodiment, the addition or presence of 10 wt. % of alcohol to a mixture comprising (a) and (b) gives a viscosity reduction of 50%.

In an embodiment, the addition or presence of at least 10 wt. % alcohol to a mixture comprising (a) and (b) gives a viscosity reduction of at least 50% (e.g., at least 51, 52, 53, 54, 55, 56, 57, 58, 59, or 60% viscosity reduction). In an embodiment, the addition or presence of at least 2.5% wt. % alcohol to a mixture comprising (a), (b) and at least 10 wt. % alcohol, gives a further viscosity reduction at least 50% (e.g., at least 51, 52, 53, 54, 55, 56, 57, 58, 59, or 60% viscosity reduction). In an embodiment, the addition or presence of at least 3.5 wt. % alcohol to a mixture comprising (a), (b) and at least 12.5 wt. % alcohol, gives a further viscosity reduction at least 50% (e.g., at least 51, 52, 53, 54, 55, 56, 57, 58, 59, or 60% viscosity reduction).

The amount of component (c) in the pharmaceutical product can have a considerable effect upon several features. In particular, the viscosity and the rate (and duration) of release will alter with the alcohol level. The amount of alcohol will thus be at least sufficient to provide a low viscosity mixture which can easily and comfortably be injected by patient applying manual pressure to an injection device but will additionally be determined so as to provide the release rate.

Physical attributes such as viscosity, and reproducibility, e.g., batch-to-batch, or injection-to-injection reproducibility, are important when the pharmaceutical product is administered using mechanical or other powered devices, e.g., devices that assist injection by application of forces higher than a manually applied force, e.g., auto-injectors. The consistency of alcohol content, e.g., ethanol, within and between lots is important for reproducible performance, e.g., optimized pharmacokinetics, when the pharmaceutical product is administered using such devices, e.g., auto-injectors.

Typically, a level of 0.1-35 wt. %, or 5-20 wt. % alcohol will provide suitable release and viscosity properties. The amount of alcohol will also be at least sufficient to ensure solubility of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection. In an embodiment, the pharmaceutical product comprises an amount of component (c), e.g., ethanol, that is sufficient to provide a solubility of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, of at least 10 mg/g, 20 mg/g, 30 mg/g, 40 mg/g, 50 mg/g or higher. In an embodiment, the pharmaceutical product comprises an amount of component (c), e.g., ethanol, which is sufficient to provide a solubility of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, of at least 30 mg/g.

In an embodiment, the amount of component (c) in the pharmaceutical product is sufficiently low that upon injection, the initial burst of drug after subcutaneous injection gives a ratio of maximum concentration (Cₘₐₓ) in plasma to minimum concentration (Cₘᵢₙ) in plasma before a next dose is administered, of less than 8, (e.g., less than 7, 6.5, 6, 5.5, 5, 4.5, 4, 3.5, 3, 2.5, 2 or lesser).

In an embodiment, the pharmaceutical product comprises 0.1-35 wt. %, 5-20 wt. %, 8-15 wt. %, or 9-11 wt. % component (c). In an embodiment, the pharmaceutical product comprises 20, 15, 14, 13, 12, 11, 10, 9, 8, 7, 6, 5 wt. % or lesser component (c), wherein the alcohol of component (c) is ethanol. In an embodiment, the pharmaceutical product comprises 10 wt. % component (c), wherein the alcohol of component (c) is ethanol.

The amount of component (c) in the pharmaceutical product described herein is sufficient to provide a low viscosity mixture (e.g., a molecular solution) of components (a), (b), (c) and (d), and may be determined for any particular combination of components by standard methods.

In an embodiment, component (c) comprises less than 5 wt. % (e.g. less than 5, 4, 3, 2, 1 wt. % or lesser) halogen substituted hydrocarbons which have lower biocompatibility.

The phase behavior may be analyzed by techniques such as visual observation in combination with polarized light microscopy, X-ray scattering and diffraction techniques, nuclear magnetic resonance, and cryo-transmission electron microscopy (cryo-TEM) to look for molecular solutions, L₂ or L₃ phases, or liquid crystalline phases or as in the case of cryoTEM, dispersed fragments of such phases. Viscosity may be measured directly by standard means. In an embodiment, an appropriate practical viscosity is that which can allow effective pressure to be applied, e.g., on a device, e.g., a syringe, e.g., a manual syringe (e.g., by manual pressure), or a spring-loaded syringe (e.g., an auto-injector) containing the pharmaceutical product. In an embodiment, a pharmaceutical product with appropriate practical viscosity allows pressure to be applied to a device, e.g., a syringe with an attached needle, e.g., a needle with a small diameter suitable for injection (e.g., a 27 Gauge needle), to administer, e.g., release, the pharmaceutical product. In an embodiment, a pharmaceutical product with appropriate practical viscosity can be sterile filtered, e.g., through filters of pore size 0.2 micron and smaller.

In an embodiment, component (c) comprises a single alcohol or a mixture of alcohols, e.g., alcohols which have a low viscosity. In an embodiment, formulations provided herein are of low viscosity and a primary role of a suitable solvent is to reduce this viscosity. This reduction will be a combination of the effect of the lower viscosity of the solvent and the effect of the molecular interactions between solvent and lipid composition. Disclosed herein is the observation that the oxygen-containing solvents of low viscosity described herein have advantageous and molecular interactions with the lipid parts of the composition, thereby providing a non-linear reduction in viscosity with the addition of a small volume of solvent.

In an embodiment, the viscosity of the low viscosity alcohol of component (c) (single solvent or mixture) is no more than 18 mPas at 20° C., e.g., no more than 15 mPas, 10 mPas, or 7 mPas.

In an embodiment, the pharmaceutical product comprises components (a), (b) and (c), wherein component (a) is GDO, component (b) is soybean PC, and component (c) is ethanol. In an embodiment, the pharmaceutical product comprises 42 wt. % of component (a), 42 wt. % of component (b) and 10 wt. % of component (c), wherein component (a) is GDO, component (b) is soybean PC, and component (c) is ethanol.

### Component (d) —Polar Solvent or Buffer

In an embodiment, the use of an alcohol in combination with a polar solvent such as a diol or water or an aqueous solution, e.g., an aqueous buffer, e.g., citrate buffer, allows an improvement in the performance of lipid-based controlled-release compositions. The addition of a diol, such as propylene glycol or water has been observed to reduce the viscosity of a lipid/alcohol/setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; pharmaceutical product without adversely affecting the release profile of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, and/or allows the proportion of alcohol to be increased without adversely affecting the release profile and/or allows an improvement in the release profile. When used herein, “adversely affecting the release profile” indicates that the ratio of Cₘₐₓ/Cₐᵥₑ is increased and/or the ratio of Cₘₐₓ/Cₘᵢₙ is increased e.g., increased by a factor of at least 1.2. Similarly, when used herein, “improving the release profile” indicates that the ratio of Cₘₐₓ/Cₐᵥₑ and/or Cₘₐₓ/Cₘᵢₙ is decreased, e.g., decreased by a factor of at least 1.2.

In an embodiment, the inclusion of a polar solvent e.g., a polar solvent comprising water, allows improvements in controlling the initial release, and allows higher stable loading of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection. In an embodiment, the inclusion of a polar solvent e.g., a polar solvent comprising water, provides faster depot formation and/or provides further reduced discomfort upon injection. Any one of these factors provides a significant improvement in the context of therapeutic drug delivery, patient health and/or patient compliance.

Component (d) as described herein comprises a polar solvent which stabilizes the pH of the pharmaceutical product, e.g., a solvent comprising water, and optionally, comprising pH modifying or buffering compounds. In an embodiment, the polar solvent of component (d) is chosen from: a histidine buffer, a citrate buffer, a succinate buffer, an acetate buffer or a phosphate buffer, or mixtures thereof. In an embodiment, component (d) comprises a citrate buffer, L-histidine buffer, or mixtures of L-histidine and L-histidine hydrochloride buffers. In an embodiment, component (d) is a citrate buffer.

In an embodiment, the citrate buffer of component (d) comprises a pH in the range of 5.8 to 7, e.g., a pH of 5.8, 5.9, 6.0, 6.1, 6.2, 6.3, 6.4, 6.5, 6.6, 6.7, 6.8, 6.9, or 7.0. In an embodiment, the citrate buffer of component (d) comprises a pH of 6.4.

In an embodiment, the pharmaceutical product comprises at least 10 wt. % (e.g., at least 10, 9, 8, 7, 6, 5, 4, 3, 2, 1, 0.5 wt. % or lesser) component (d), wherein component (d) is a buffer, e.g., a citrate buffer at pH 6.4. In an embodiment the pharmaceutical product comprises component (d) in the ranges of 0.5-10 wt. %, 1-5 wt. %, or 1-3 wt. %, wherein component (d) is a buffer, e.g., a citrate buffer at pH 6.4. In an embodiment, the pharmaceutical product comprises 2 wt. % component (d), wherein component (d) is a buffer, e.g., a citrate buffer at pH 6.4.

In an embodiment, the citrate buffer of component (d) comprises citric acid monohydrate and optionally, an antioxidant, e.g., disodium edetate (EDTA disodium salt). In an embodiment, component (d) comprises a solution of 30 mM citric acid monohydrate. e.g., 30 mM, 25 mM, 24 mM, 23 mM, 22 mM, 21 mM, 20 mM, 15 mM, 10 mM, or 5 mM citric acid monohydrate. In an embodiment, component (d) comprises a solution of 23 mM citric acid monohydrate.

In an embodiment, the citrate buffer of component (d) comprises citric acid monohydrate and an antioxidant, e.g., wherein the antioxidant is disodium edetate (EDTA). In an embodiment, component (d) comprises a solution of 30 mM citric acid monohydrate (e.g., 30, 25, 24, 23, 22, 21, 20, 15, 10, or 5 mM citric acid monohydrate) and a solution of 350 μM disodium edetate (e.g., 350, 340, 330, 320, 310, 300, 290, 280, 270, 260, 250, 200, 100, or 50 uM disodium edetate). In an embodiment, component (d) comprises a solution of 23 mM citric acid monohydrate and a solution of 300 uM disodium edetate.

In an embodiment, component (d) comprises an optional component, e.g., an antioxidant or a chemical or physical stabilizing agent chosen from EDTA (e.g., edetate, with a pharmaceutically acceptable salt such as but not limited to sodium, calcium, magnesium), BHA (Butylated Hydroxyanisole), BHT (Butylated Hydroxytoluene), Ascorbic acid, Alpha Tocopherol (Vitamin-E), or thiosulfate (with a pharmaceutically acceptable salt such as but not limited to sodium or calcium).

In an embodiment, the optional component of component (d), e.g., an antioxidant or a chemical or physical stabilizing agent, can stabilize and suppress the oxidative degradation of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, described herein. In an embodiment, the optional component of component (d), e.g., an antioxidant or a chemical or physical stabilizing agent, does not reduce the solubility of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; or reduce the clarity, e.g., the clarity of solution, of the pharmaceutical product. In an embodiment, the optional component of component (d), e.g., an antioxidant or a chemical or physical stabilizing agent, does not reduce the ability of the pharmaceutical product to form a structured gel like structure upon injection, or in contact with simulated physiological fluids.

In an embodiment, the antioxidant of component (d) increases the physical and chemical stability of the dissolved or dispersed setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient, as the sole active ingredient for injection, as the active pharmaceutical ingredient, or as the active pharmaceutical ingredient for injection resulting in a greater amount of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient, as the sole active ingredient for injection, as the active pharmaceutical ingredient, or as the active pharmaceutical ingredient for injection in the presence of the antioxidant. In an embodiment, the antioxidant of component (d) increases the chemical and/or physical stability of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient, as the sole active ingredient for injection, as the active pharmaceutical ingredient, or as the active pharmaceutical ingredient for injection in a lipid formulation under conditions of typical storage, e.g., 0-5° C., or 20-25° C.

By inclusion of some polar solvent which is miscible with the alcohol component (especially water), the slight sensation that may be caused at the injection site from the alcohol content can be substantially eliminated. In an embodiment, the pharmaceutical product comprises a ratio of components (c):(d) between 10:90 to 90:10, 20:80 to 80:20, 30:70 to 70:30, or 40:60 to 60:40.

In an embodiment, the pharmaceutical product comprises components (c) and (d), wherein component (c) comprises ethanol (e.g., at least 20, 15, 14, 13, 12, 11, 10, 9, 8, 7, 6, 5 wt. % or lesser of ethanol) and component (d) comprises a citrate buffer optionally comprising an antioxidant (e.g., at least 10, 9, 8, 7, 6, 5, 4, 3, 2, 1, 0.5 wt. % or lesser of a citrate buffer, optionally comprising an antioxidant). In an embodiment, the formulation comprises 10% of ethanol and 2% of a citrate buffer, wherein the buffer optionally comprises an antioxidant.

In an embodiment, the pharmaceutical product described herein comprises, GDO (e.g., 40-70 wt. % GDO), soybean PC (e.g., 40-70 wt. % GDO), ethanol (e.g., 5-20 wt. % ethanol), and a citrate buffer comprising disodium edetate (e.g., 0.5-10 wt. % of 23 mM citrate buffer comprising 300 mM disodium edetate), or mixtures thereof. In an embodiment, the pharmaceutical product comprises 42 wt. % GDO, 42 wt. % soybean PC, 10 wt. % ethanol, 2 wt. % of 23 mM citrate buffer comprising 300 mM disodium edetate, or mixtures thereof.

### Component (e)

Component (e) as described herein comprises setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-2_2511, BIM-22287, BIM-22512, 001152C, 001543C. 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection. In an embodiment, component e) comprises setmelanotide.

In any of the embodiments described herein, component (e) comprises setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient, as the sole active ingredient for injection, as the active pharmaceutical ingredient, or as the active pharmaceutical ingredient for injection in a suitable salt or gel form. In an embodiment, the salt form is a pharmaceutically acceptable salt. Exemplary salts (e.g., pharmaceutically acceptable salts) include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, triethiodide, and trifluoroacetate salts. In an embodiment, component (e) comprises setmelanotide in a suitable salt, e.g., a chloride salt. In an embodiment, component (e) comprises a chloride salt of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., B1M-22511, BIM-22287, BIM-212512, 001152C, 001543C, 001003C, 001574C, 001555C, 001554C, 001556C, 001358C, 001576C1, 001364C, 001258C, or MC4R-11) as the sole active ingredient, as the sole active ingredient for injection, as the active pharmaceutical ingredient, or as the active pharmaceutical ingredient for injection. In an embodiment, component (e) comprises a chloride salt of setmelanotide.

In an embodiment, component (e) comprises an MC4RA_{P} (e.g., B-22511, HIM-22287, BIM-22512, 001152C, 001543C, 001003C, 001574C, 001555C, 001554C, 001556C, 00138C, 001576C, 001364C, 001258C or MC4R-11). Structures of exemplary MC4RA_{P} compounds are described in Table 1.

TABLE 1
Structure of MC4RA_{P} compounds

| ID | Structure |
|---|---|
| BIM-22511 | c(Hydantoin(Arg-Gly))-c[Cys-Glu-His-D-Phe-Arg-Trp-Cys]-NH2 |
| BIM-22287 | c(Hydantoin(C(O)-(Nle-Gly))-c[Cys-Glu-His-D-Phe-Arg-Trp-Cys]-NH2 |
| BIM-22512 | c(Hydantoin(C(O)-(Gly-Arg))-c[Cys-Glu-His-D-Phe-Arg-Trp-Cys]-NH2 |
| MC4R-11 | c(Hydantoin(C(O)-(Glu-D-Ala))-His-D-Phe-Arg-Trp-Apr]-NH2 |
| 001152C | Ac-Arg-c[Cys-D-Ala-Gln-D-Phe-Arg-Trp-Cys]-NH2 |
| 001543C | Ac-Arg-c[Cys-Ala-Gln-D-Phe-Arg-Trp-Cys]-NH2 |
| 001003C | Ac-Arg-c[Cys-D-Ala-His-D-Phe(4F)-Arg-Trp-Cys]-NH2 |
| 001574C | Ac-Arg-c[Hcys-Thr-D-Phe-Arg-Trp-Pen]-NH2 |
| 001555C | Ac-Arg-c[Hcys-Gln-D-Phe-Arg-Trp-Pen]-NH2 |
| 001554C | Ac-Arg-c[Hcys-Asn-D-Phe-Arg-Trp-Pen]-NH2 |
| 001556C | Ac-Arg-c[Hcys-Ser-D-Phe-Arg-Trp-Pen]-NH2 |
| 001358C | Ac-Arg-c[Hcys-Ala-D-Phe-Arg-Trp-Pen]-NH2 |
| 001576C | Ac-Arg-c[Glu-Gln-D-Phe-Arg-Trp-Apr]-NH2 |
| 001364C | Ac-Arg-c[Hcys-Val-D-Phe-Arg-Trp-Cys]-NH2 |
| 001258C | Ac-Arg-c[Glu-Ala-D-Phe-Arg-Trp-Apr]-NH2 |

In an embodiment, component (e) comprises an MC4RA_{P}, e.g., a compound comprising SEQ ID NO: 11, as described in International Application WO 2008/147556, the entire contents of which are hereby incorporated by reference. In an embodiment, a compound comprising SEQ ID NO:11 is referred to as MC4R-11 herein.

In an embodiment, examples of compounds comprising SEQ ID NO:11, e.g., MC4R-11, include:
cyclo[Hydantoin(C(O)-(Glu-D-Ala))-His-D-Phe-Arg-Trp-Orn]-NH2;
cyclo[Hydantoin(C(O)-(Glu-D-Ala))-His-D-Phe-Arg-Trp-Dab]-NH2; or cyclo[Hydantoin(C(O)-(Glu-D-Ala))-His-D-Phe-Arg-Trp-Dap]-NH2;
or a pharmaceutically acceptable salt thereof, particularly cyclo[Hydantoin(C(O)-(Glu-D-Ala))-His-D-Phe-Arg-Trp-Dap]-NH2.

In an embodiment, a compound comprising SEQ ID NO: 11, also known as MC4R-11, comprises the following amino acid sequence:
HXRWX (SEQ ID NO: 11), wherein X can be chosen from: Ornithine (Orn); 2,4-diaminobutyric acid (Dab); or 2,3-diaminoproprionic acid (Dap).

In an embodiment, the pharmaceutical product comprises 0.02-12 wt. % of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection. In an embodiment, the pharmaceutical product comprises 0.02-12 wt. % of setmelanotide.

In an embodiment, the pharmaceutical product comprises 0.1-10 wt. %. 0.2-8 wt. %, 0.5-6 wt. %, 1-4 wt. % setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection. In an embodiment, the pharmaceutical product comprises 0.1-10 wt. %, 0.2-8 wt. %, 0.5-6 wt. %, 1-4 wt. % setmelanotide.

In an embodiment, the pharmaceutical product comprises about 1 mg/mL, 2 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mil, 55 mg/mL, 60 mg/mL, 65 mg/mL, 70 mg/mL, 75 mg/mL or more of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287. BIM-22512, 001152C, 001543C, 001003C, 001574C, 0.001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.

In an embodiment, the pharmaceutical product comprises about 5 mg/mL setmelanotide. In an embodiment, the pharmaceutical product comprises about 10 mg/mL setmelanotide. In an embodiment, the pharmaceutical product comprises about 20 mg/mL setmelanotide. In an embodiment, the pharmaceutical product comprises about 25 mg/mL setmelanotide. In an embodiment, the pharmaceutical product comprises about 30 mg/ml setmelanotide. In an embodiment, the pharmaceutical product comprises about 35 mg/mL setmelanotide. In an embodiment, the pharmaceutical product comprises about 40 mg/mL setmelanotide.

In an embodiment, the pharmaceutical product comprises more than about 1 mg/mL, 2 mg/mL, 5 mg/mL, 10 mg/mL, 15 mg/ml, 20 mg/ml, 25 mg/mL, 30 mg/ml, 35 mg/mL, 40 mg/mL, 45 mg/mL, 50 mg/mL, 55 mg/mL, 60 mg/mL, 65 mg/mL, 70 mg/mL, 75 mg/mL or more setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.

In an embodiment, the pharmaceutical product comprises less than about 50 mg/nL, 40 mg/ml, 35 mg/mL, 30 mg/mL, 25 mg/ml, 20 mg/mL, 15 mg/mL, 10 mg/mL, 5 mg/mL, 2 mg/mL, or 1 mg/mL setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.

In an embodiment, the pharmaceutical product comprises 3 wt. % of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection. In an embodiment, the pharmaceutical product comprises 3 wt. % of setmelanotide.

In an embodiment, the pharmaceutical product comprising 3 wt. % of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; is stable to storage without loss or degradation of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection at 25° C. for at least 1-6 months, e.g., for at least 1, 2, 3, 4, 5, 6 months or longer. In an embodiment the pharmaceutical product comprises 3 wt. % setmelanotide.

In an embodiment a pharmaceutical product, e.g., a formulation comprising 3 wt. % of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection is stable to storage without loss or degradation of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection at 5° C. for at least 1-6 months, e.g., for at least 1, 2, 3, 4, 5, 6 months or longer. In an embodiment the pharmaceutical product comprises 3 wt. % setmelanotide.

In an embodiment, the pharmaceutical product comprising about 1 mg/mL, 2 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL or more of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection is stable to storage without loss or degradation of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection at 25° C. for at least 1-6 months, e.g., for at least 1, 2, 3, 4, 5, 6 months or longer.

In an embodiment, the pharmaceutical product comprising about 1 mg/mL, 2 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL. 75 mg/mL or more of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection is stable to storage without loss or degradation of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIN-22511, BIM-22287, BIM-22512, 001152C, 001543C, 001003C, 001574C, 001555C, 001554C, 001556C, 001358C, 001.576C, 001.364C, 001258C, or MC4R-11), as the sole active ingredient, as the sole active ingredient for injection, as the active pharmaceutical ingredient, or as the active pharmaceutical ingredient for injection at 5° C. for at least 1-6 months, e.g., for at least 1, 2, 3, 4, 5, 6 months or longer.

In an embodiment wherein setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., B1M-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is highly soluble in the alcohol of component (c), the solubility of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, in the alcohol is controlled.

While not wishing to be bound by theory, it is believed that in an embodiment, the excessive solubility of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287. BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection in the alcohol of component (c) results in the alcohol transporting a significant quantity of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, out of the depot composition as it forms in viva. In one embodiment, the polar solvent of the pharmaceutical product is used to control the solubility of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, in the pharmaceutical product so as to aid control of the release profile. In an embodiment, the pharmaceutical product comprises setmelanotide.

In an embodiment, the disclosure provides a method for controlling the solubility of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, in a low viscosity mixture comprising: (a) 40-70 wt, % of a diacyl glycerol and/or a tocopherol; (b) 40-70 wt. % of a phospholipid; (c) 5-20 wt. % of an alcohol; (e) 1-4 wt. % of a peptide; and (d) 0.5-10 wt. % of a polar solvent, optionally comprising an antioxidant, to form a depot pharmaceutical product. In an embodiment, the pharmaceutical product comprises setmelanotide.

In an embodiment, the disclosure provides a method for modulating the release profile of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, from a depot composition formed by injection of a low viscosity mixture comprising: (a) 40-70 wt. % of a diacyl glycerol and/or a tocopherol; (b) 40-70 wt. % of a phospholipid; (c) 5-20 wt. % of an alcohol; (e) 1-4 wt. % of setmelanotide; and (d) 0.5-10 wt, % of a polar solvent, optionally comprising an antioxidant, in said low-viscosity mixture to form a depot pharmaceutical product. In an embodiment, the pharmaceutical product comprises setmelanotide.

In an embodiment, the disclosure provides a method and use for the reduction of injection-site discomfort, reduction of viscosity of the pharmaceutical product, and/or reduction in initial burst release of a low viscosity mixture comprising: (a) 40-70 wt. % of a diacyl glycerol and/or a tocopherol; (b) 40-70 wt. % of a phospholipid; (c) 5-20 wt. % of an alcohol; (e) 1-4 wt. % of setmelanotide; and (d) 0.5-10 wt. % of a polar solvent, optionally comprising an antioxidant, in said low-viscosity mixture to form a depot of the pharmaceutical product. In an embodiment, the pharmaceutical product comprises setmelanotide.

In an embodiment, the disclosure provides a method and a use for improving the properties of the pharmaceutical product and/or the resulting depot composition without negatively affecting the release profile of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287. BIM-22512, 001152C. 001543C. 001003C. 001574C. 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection. In an embodiment, the pharmaceutical product comprises setmelanotide.

### Optional Additional Components

In an embodiment, the pharmaceutical product disclosed herein does not include (or includes less than 5, 2, 1, or 0.5% by weight) fragmentation agents, such as polyethyleneoxide or poly(ethylene glycol) (PEG) fragmentation agent, e.g., a PEG grafted lipid and/or surfactant.

In an embodiment, pharmaceutical product disclosed herein does not include (or includes less than 5, 2, 1, or 0.5% by weight) fragmentation agents such as Polysorbate 80 (P80), or other Polysorbates (e.g. Polysorbate 20), PEGylated phospholipids (PEG-lipids such as DSPE-PEG(2000), DSPE-PEG(5000), DOPE-PEG(2000) and DOPE-PEG(5000)), Solutol HS 15, PEGylated fatty acids (e.g. PEG-oleate), block co-polymers such as Pluronic.RTM. F127 and Pluronic.RTM. F68, ethoxylated castor oil derivatives (e.g. Chremophores), PEGylated glyceryl fatty acid esters (such as TMGO-15 from Nikko Chemicals) and PEGylated tocopherols (such as d-alpha tocopheryl poly(ethylene glycol)1000 succinate known as Vitamin E TPGS from Eastman.

In an embodiment, setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is a powder, e.g., in a kit. In an embodiment, the kit comprises setmelanotide.

In an embodiment, setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is dissolved in a lipid pharmaceutical product, and gains stability, e.g., storage and in vivo stability, by certain stabilizing additives, e.g., additives comprising sugars (e.g., sucrose, trehalose, or lactose), polymers (e.g., polyols such as carboxy methyl cellulose), amino acids (e.g., methionine, glutamate, or lysine), lipid-soluble acid components (e.g., HCl), anionic lipids or surface active agents (e.g., dioleoyl phosphatidyl glycerol (DOPG), palmitoyloleoyl phosphatidylglycerol (POPG) or oleic acid (OA)). In some embodiments, the lipid pharmaceutical product comprises setmelanotide.

In an embodiment, the pharmaceutical product described herein comprises a single dose format or multi-dose formats. In an embodiment, a single dose format or multi-dose format of the pharmaceutical product can be stored in a pharmaceutically acceptable glass or plastic container. In an embodiment, single-dose formats of the pharmaceutical product remain stable, e.g., chemically and physically stable, and potent in storage prior to use, but are disposable after the single use. In one embodiment, a single dose format or multi-dose format of the pharmaceutical product is stable at 4° C., e.g., at 0, 1, 2, 3, or 4° C., for at least 36 months, e.g., at least 1, 2, 3, 4, 5, 6, 7, 8, 9, 10, 11, 12, 15, 20, 25, 30, or 36 months. In an embodiment, a single dose format or multi-dose format of the pharmaceutical product is stable at 30° C., e.g., at 20, 21, 22, 23, 24, 25, 26, 27, 28, 29, or 30° C., for at least 36 months, e.g., at least 1, 2, 3, 4, 5, 6, 7, 8, 9, 10, 11, 12, 15, 20, 25, 30, or 36 months. In an embodiment, multi-dose formats of the pharmaceutical product remain stable, e.g., chemically and physically stable, and potent in storage prior to use, and also remain stable, e.g., chemically and physically stable, potent and relatively free of bacteria over the multiple-dose use regimen administration period after the first use in which a seal has been compromised. In an embodiment, multi-dose formats of the pharmaceutical product comprise an anti-microbial or microbial-static agent, e.g., bacteriostatic agent or preservative.

In an embodiment, the pharmaceutical product described herein optionally comprises an antimicrobial or microbial-static agent, e.g., bacteriostatic agents or preservatives, with sufficient antimicrobial activity to meet European Pharmacopoeia (Ph. Eur.), Japanese Pharmacopoeia (JP) and United States Pharmacopoeia (USP) standards. In an embodiment, the bacteriostatic agents or preservatives allow the pharmaceutical product to be stored and used for multiple doses. In an embodiment, the bacteriostatic agents or preservatives do not reduce the solubility of the setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, or reduce the clarity, e.g., clarity of the solution, of the pharmaceutical product. In an embodiment, the bacteriostatic agents or preservatives comprising the pharmaceutical product are chosen from phenol, benzoic acid, chlorobutanol, benzalkonium chloride, m-cresol, p-cresol, benzyl alcohol or other phenolic preservatives. In an embodiment, the bacteriostatic agents or preservatives are used at concentrations wherein the bacteriostatic agents or preservatives are effective. In an embodiment, the bacteriostatic agents or preservatives comprising the pharmaceutical product are pharmaceutically accepted for use by administration by injection.

In an embodiment, the pharmaceutical product described herein optionally comprises an antimicrobial or microbial-static agent in addition to an antioxidant or a chemical or physical stabilizing agent, e.g., EDTA.

In an embodiment, the pharmaceutical product comprising a setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, described herein does not require an additional preservative, anti-microbial or microbial-static agent, e.g., bacteriostatic or bacteriocide, to provide a multi-use format. In an embodiment, the pharmaceutical product comprises setmelanotide.

In an embodiment, the pharmaceutical product described herein provides a preservative effect with an acceptable stability of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, and pharmaceutical product stability, and can be used for single-dose as well as multiple-dose use. In an embodiment, the pharmaceutical product comprises setmelanotide.

In an embodiment, the pharmaceutical product described herein for multi-use format comprise ethanol and citrate buffer, optionally comprising disodium edetate, in sufficient concentrations either alone or in any combination, to provide a preservative effect while maintaining stability of the setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, and the pharmaceutical product. In an embodiment, the pharmaceutical product comprises setmelanotide.

### Method of Making—Specified Order

The order in which the components of a pharmaceutical product of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, are combined can optimize the method of making, e.g., by decreasing the time it takes to solubilize setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection. In an embodiment, the pharmaceutical product comprises setmelanotide.

In an embodiment, a substantial portion, e.g., all of component a, b, or both a and b, are added after setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is contacted with, e.g., dissolved or dispersed in, an alcohol. In an embodiment, setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is dissolved or dispersed in an alcohol, e.g., ethanol. Once all or some, e.g., at least 50, 70, 80, 90, 95, or 99%, of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is dissolved or dispersed in the ethanol, a substantial portion, e.g., all of component a, b, or both a and b, are added to the mixture. In an embodiment, the mixture can also contain component d, e.g., a polar solvent, e.g., a citrate buffer, e.g., at the time setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is dissolved or dispersed in an alcohol, e.g., when at least 50, 70, 80, 90, 95, or 99%, of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is dissolved or dispersed in the ethanol. In an embodiment, the pharmaceutical product comprises setmelanotide.

In an embodiment, setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is contacted with one or more components a, b, and d after setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is dissolved or dispersed in an alcohol. In an embodiment, the pharmaceutical product comprises setmelanotide.

### Method of Making—Controlling the Amount of Ethanol

It can be important to have a biologically active moiety, e.g., setmelanotide, within a pharmaceutical product that contains a predetermined or specified level of alcohol, e.g., a level that optimizes the viscosity of the pharmaceutical product prior to injections and the release profile after injection. Manufacturing methods can allow uncontrolled or unpredictable levels of loss of alcohol, e.g., by evaporation, during manufacture, and this loss may impede the ability to have controlled and repeatable levels of alcohol in the formulation. Methods of making a pharmaceutical product comprising a setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, having controlled and repeatable levels of alcohol, e.g., ethanol, are provided herein. The level of alcohol affects both pre-delivery viscosity and post-delivery release profile. Alcohol generally promotes lower viscosity but if too much is present it can affect release profiles in undesirable ways, e.g., by increasing Cₘₐₓ. Thus, a controlled and repeatable level of alcohol is desirable.

Methods described herein control the level of alcohol in the completed pharmaceutical product, e.g., a pharmaceutical product comprising setmelanotide. Loss of added alcohol by evaporation can result in variability in the level of alcohol present in the pharmaceutical product. In an embodiment, the method comprises producing the pharmaceutical product in a closed system or vessel with the addition of a controlled amount of ethanol. Using a closed system or vessel may minimize loss of alcohol from evaporation.

In an embodiment, the disclosure provides a method of making the pharmaceutical product described herein with a controlled, or predetermined, amount of alcohol, e.g., ethanol. In an embodiment, the amount of alcohol, e.g., ethanol, in the pharmaceutical product is a predetermined amount, e.g., 10%. Methods of the disclosure comprise adding a measured amount of alcohol, e.g., ethanol, sometime referred to herein as the “addition amount” that, under the manufacturing conditions, results in a pharmaceutical product having a predetermined amount of alcohol, e.g., ethanol, after manufacture. Generally, the addition amount will be greater than the predetermined amount present after manufacturing, e.g., due to evaporation of alcohol during manufacturing. In an embodiment the addition amount is added as a single aliquot, and in other embodiments a plurality of aliquots are added to provide the addition amount. In embodiments, the method comprises additional measures that reduce alcohol evaporation, e.g., conducting one or more phases of the manufacturing process under conditions that reduce alcohol evaporation, e.g., controlling temperature or conducting a phase of the process in a closed vessel.

In an embodiment, the alcohol, e.g., ethanol, can be provided to the pharmaceutical product in one or more than one amounts or aliquots, e.g., in one, two, three or four amounts or aliquots. In an embodiment, a method of making the pharmaceutical product comprises, providing a first amount or aliquot of alcohol, e.g., ethanol, to setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, to dissolve or disperse the peptide. In an embodiment, a method of making the pharmaceutical product further comprises providing a substantial portion of components a, b and d, or all of components a, b, and d to the mixture of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, dissolved or dispersed in ethanol. In an embodiment, the pharmaceutical product comprises setmelanotide.

In an embodiment, a method of making the pharmaceutical product further comprises providing a next amount or aliquot, e.g., a second, third or fourth amount or aliquot of alcohol, e.g., ethanol, to the mixture thereby making a pharmaceutical product of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, comprising 10 wt. % alcohol, e.g., ethanol.

Methods of Treatment with Formulations

Methods of administering the pharmaceutical product described herein are described, e.g., in International Applications WO 2013/102047, WO 2014/144842, and WO 2017/059076, the entire contents of all of which are hereby incorporated by reference in their entirety.

In embodiments, the pharmaceutical product comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, as described herein is administered to a subject having a disease or disorder that is responsive to the pharmacological activity possessed by the polypeptide of the pharmaceutical product, e.g., setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection. In one embodiment the pharmaceutical product comprises setmelanotide.

In one embodiment, the disorder to be treated is responsive to the modulation of MC4R in a subject in need of treatment. The method comprises administering to the subject an effective amount of a pharmaceutical product comprising as the polypeptide an MC4R modulator, e.g., setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection. In an embodiment, the pharmaceutical product comprises setmelanotide.

In an embodiment, the disorder responsive to modulation of the MC4R includes type 1 diabetes, type 2 diabetes, obesity, insulin resistance, metabolic syndrome, male erectile dysfunction, female sexual disorder, non-alcoholic fatty liver disease, non-alcoholic steatohepatitis, disorders of substance abuse, including alcoholism, feeding disorders, cachexia, inflammation and anxiety. In an embodiment, the disorder is obesity, type 1 diabetes or type 2 diabetes. In an embodiment, the disorder is obesity or an obesity-related condition. In an embodiment, the disorder is Prader-Willi Syndrome. In an embodiment, the disorder is Bardet-Biedl syndrome. In an embodiment, the disorder is Alström syndrome.

In an embodiment, the disorder to be treated comprises type 1 diabetes, type 2 diabetes, obesity, Prader-Willi Syndrome, insulin resistance, metabolic syndrome, male erectile dysfunction, female sexual disorder, non-alcoholic fatty liver disease, non-alcoholic steatohepatitis, disorders of substance abuse, including alcoholism, feeding disorders, cachexia, inflammation, anxiety, Bardet-Biedl syndrome, or Alström syndrome. In an embodiment, the disorder is obesity, type 1 diabetes or type 2 diabetes. In an embodiment, the disorder is obesity or an obesity-related condition. In an embodiment, the disorder is Prader-Willi Syndrome. In an embodiment, the disorder is Bardet-Biedl syndrome. In an embodiment, the disorder is Alström syndrome. In an embodiment, the disorder to be treated involves a disorder related to or resulting from one or more mutations in a gene related to the leptin-melanocortin pathway or POMC-MC4R pathway, such as leptin, a leptin receptor, pro-opiomelanocortin (POMC), a prohormone convertase (e.g., PCSK1), or α-MSH.

### Definitions

“Naturally derived”, as used herein, refers to both a compound isolated from the natural source as well as to a wholly or partially synthetically synthesized compound having the same structure, or substantially the same structure, as the compound found in nature.

The compositions and methods disclosed herein encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least 85%, 90%, 91%, 92%, 93%, 94%, 95%, 96%, 97%, 98% or 99% identity to a reference sequence, e.g., a sequence provided herein.

### Formulations

A pharmaceutical product can include, e.g., setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, containing pharmaceutical product described herein, and a buffer. The pH of the formulation is generally pH 5.5-7.0.

In an embodiment, the pharmaceutical product is a liquid formulation which has a low viscosity. In an embodiment, the pharmaceutical product is formulated to be directly injected, e.g., without further additions, or adjustments before injection. In an embodiment, the pharmaceutical product is stored as a liquid in a device, e.g., a syringe, e.g., a syringe described herein. In an embodiment, the syringe can be chosen from: a manual syringe, a syringe comprising a needle (e.g., a needle with a suitable diameter, e.g., a 27 Gauge needle), a single-use syringe, a multi-use syringe, or a spring-loaded syringe (e.g., an auto-injector).

In an embodiment, components of the pharmaceutical product can be prepared as separate liquids, or solid, e.g., lyophilized, formulations, and stored separately. In an embodiment, the components of the pharmaceutical product can be prepared as at least two, e.g., 2, 3, 4, or 5, separate components. In an embodiment, components, e.g., the at least two components comprising the pharmaceutical product, can be combined, mixed, dissolved or dispersed shortly before injection. In an embodiment, components, e.g., the at least two components comprising the pharmaceutical product, are prepared and stored in separate containers, e.g., at least two separate containers, e.g., containers described herein, and mixed by combining the components, e.g., the at least two components, into one container, e.g., a container described herein. In an embodiment the components, e.g., the at least two components comprising the pharmaceutical product are combined into a receptacle (e.g., a receptacle described herein, e.g., a receptacle with more than one chamber, e.g., at least two chambers), which separates the components, e.g., the at least two components, by means of a pharmaceutically acceptable seal, e.g., maintaining the components, e.g., the at least two components, in separate chambers within the one receptacle, e.g., a receptacle described herein. In an embodiment, the seal can be manipulated, e.g., opened, or removed, to mix the components, e.g., the at least two components comprising the pharmaceutical product to form a single solution or suspension in the receptacle. In an embodiment, the solution or suspension of the pharmaceutical product can be loaded into a syringe, e.g., a syringe described herein. In an embodiment, the solution or suspension of the pharmaceutical product can be administered, e.g., in an effective amount (e.g., a therapeutically effective amount), to a subject in need thereof, e.g., a subject described herein.

In an embodiment, a container can be chosen from: a syringe (e.g., a manual syringe, a syringe comprising a needle (e.g., a needle with a suitable diameter, e.g., a 27 Gauge needle), a single-use syringe, a multi-use syringe, or a spring-loaded syringe (e.g., an auto-injector); a vial (a single-use vial, or a multi-use vial); or other pharmaceutically acceptable container.

In an embodiment, a receptacle as described herein includes, but is not limited to: Vetter Lyo-ject® dual-chamber syringe, Vetter V-LK® dual-chamber cartridge, Credence Companion® dual-chamber, or other dual-chamber devices, e.g., cartridges or syringes.

### Buffers

The term “buffer” as used herein denotes an excipient which stabilizes the pH of the pharmaceutical composition. Examples of buffers include histidine-buffers, citrate-buffers, succinate-buffers, acetate-buffers and phosphate-buffers or mixtures thereof. In an embodiment, a buffer of the pharmaceutical product comprises citrate, L-histidine, or mixtures of L-histidine and L-histidine hydrochloride. In an embodiment, a buffer of the pharmaceutical product is an acetate buffer. Independently from the buffer used, the pH can be adjusted with an acid or a base, e.g. hydrochloric acid, acetic acid, phosphoric acid, sulfuric acid and citric acid, sodium hydroxide and potassium hydroxide.

The pH of a pharmaceutical product as described herein is generally between pH 5.0 to 7.5, for example, pH 5.5 to 6.5, pH 5.5 to 6.0, pH 6.0 to 6.5, pH 6.5 to 7.0, pH 5.5, pH 6.0, pH 6.5 or pH 7.0. In an embodiment, a buffer that can maintain a solution at pH 5.5 to pH 7.0 is used to prepare a pharmaceutical product. In an embodiment, a buffer used to prepare the pharmaceutical product includes, without limitation, citric acid, HEPES, histidine, arginine, potassium acetate, potassium citrate, potassium phosphate (K₂HPO₄), sodium acetate, sodium bicarbonate, sodium citrate, sodium phosphate (NaH₂PO₄), Tris base, and Tris-HCl. In an embodiment, the concentration of the buffer is between 5 mM and 100 mM, e.g., 5 mM to 50 mM. In an embodiment, sodium phosphate buffer is used at a concentration of 25 nM. In an embodiment, a sodium citrate buffer is used at a concentration of 10 mM or 25 mM. In an embodiment, a histidine buffer is used at a concentration of 25 mM. In an embodiment, an arginine buffer is used at a concentration of 25 mM.

### NUMBERED EMBODIMENTS

The invention is further described with reference to the following numbered embodiments.

1. A composition or preparation, e.g., a composition of a pharmaceutical product, comprising:
a) a neutral diacyl lipid and/or a tocopherol;
b) a phospholipid:
c) an alcohol;
d) optionally, a polar solvent, e.g., a buffer, optionally comprising an antioxidant; and
e) setmelanotide, e.g., setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), e.g., as the sole active ingredient, as the sole active ingredient for injection, as the active pharmaceutical ingredient, or as the active pharmaceutical ingredient for injection.
   2. A composition or preparation, e.g., a composition of a pharmaceutical product, comprising:
a) a neutral diacyl lipid and/or a tocopherol;
b) a phospholipid:
c) an alcohol;
d) optionally, a polar solvent, e.g., a buffer, optionally comprising an antioxidant; and
e) setmelanotide.
   3. A composition or preparation, e.g., a composition of a pharmaceutical product, comprising:
a) a neutral diacyl lipid and/or a tocopherol;
b) a phospholipid:
c) an alcohol;
d) optionally, a polar solvent, e.g., a buffer, optionally comprising an antioxidant; and
e) setmelanotide as sole active pharmaceutical ingredient formulated for injection.
   4. A composition or preparation, e.g., a composition of a pharmaceutical product, comprising:
a) a neutral diacyl lipid and/or a tocopherol;
b) a phospholipid:
c) an alcohol;
d) optionally, a polar solvent, e.g., a buffer, optionally comprising an antioxidant; and
e) setmelanotide as the active pharmaceutical ingredient.
   5. A composition or preparation, e.g., a composition of a pharmaceutical product, comprising:
a) a neutral diacyl lipid and/or a tocopherol;
b) a phospholipid:
c) an alcohol;
d) optionally, a polar solvent, e.g., a buffer, optionally comprising an antioxidant; and
e) setmelanotide as the active pharmaceutical ingredient for injection.
   6. A composition or preparation, e.g., a composition of a pharmaceutical product, comprising:
a) a neutral diacyl lipid and/or a tocopherol;
b) a phospholipid:
c) an alcohol;
d) optionally, a polar solvent, e.g., a buffer, optionally comprising an antioxidant; and
e) an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11).
   7. A composition or preparation, e.g., a composition of a pharmaceutical product, comprising:
a) a neutral diacyl lipid and/or a tocopherol;
b) a phospholipid:
c) an alcohol;
d) optionally, a polar solvent, e.g., a buffer, optionally comprising an antioxidant; and
e) an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient.
   8. A composition or preparation, e.g., a composition of a pharmaceutical product, comprising:
a) a neutral diacyl lipid and/or a tocopherol;
b) a phospholipid:
c) an alcohol;
d) optionally, a polar solvent, e.g., a buffer, optionally comprising an antioxidant; and
e) an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the sole active ingredient for injection.
   9. A composition or preparation, e.g., a composition of a pharmaceutical product, comprising:
a) a neutral diacyl lipid and/or a tocopherol;
b) a phospholipid:
c) an alcohol;
d) optionally, a polar solvent, e.g., a buffer, optionally comprising an antioxidant; and
e) an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the active pharmaceutical ingredient.
   10. A composition or preparation, e.g., a composition of a pharmaceutical product, comprising:
a) a neutral diacyl lipid and/or a tocopherol;
b) a phospholipid:
c) an alcohol;
d) optionally, a polar solvent, e.g., a buffer, optionally comprising an antioxidant; and
e) an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) as the active pharmaceutical ingredient for injection.
   11. The composition of any one of the preceding embodiments, comprising a neutral diacyl lipid.
   12. The composition of embodiment 11, wherein the neutral diacyl lipid comprises diacyl glycerol.
   13. The composition of any of embodiments 11-12, wherein the neutral diacyl lipid comprises glycerol dioleate (GDO).
   14. The composition of any one of the preceding embodiments, wherein the phospholipid comprises phosphatidylcholine.
   15. The composition of any one of the preceding embodiments, wherein the phospholipid comprises soybean phosphatidylcholine.
   16. The composition of any one of the preceding embodiments, wherein the alcohol comprises ethanol.
   17. The composition of any one of the preceding embodiments, wherein the ethanol is provided in an amount that is sufficiently great that it provides a solubility of setmelanotide of at least 10 mg/g, 20 mg/g or 30 mg/g.
   18. The composition of embodiment 17, wherein the amount by weight % of ethanol is greater than 1% by weight, e.g., between 1-20% by weight.
   19. The composition of any of embodiments 17-18, wherein the amount of ethanol is sufficiently low that injection can be made easily and comfortably by operation of a device, e.g., a syringe, by a subject, e.g., a subject described herein.
   20. The composition of any of embodiments 17-19, wherein the amount, by weight % of ethanol is less than 20%, 15%, or 10%.
   21. The composition of any of embodiments 17-20, wherein, the amount, of ethanol is sufficiently great that the composition has a viscosity low enough to be comfortably delivered with a device, e.g., a syringe with a narrow needle, e.g., a 27 Gauge needle.
   22. The composition of any of embodiments 17-21, wherein the amount of ethanol is sufficiently low that, upon injection, the initial burst of drug, e.g., initial release, after subcutaneous injection gives a ratio of maximum concentration (Cₘₐₓ) in plasma to minimum concentration (Cₘᵢₙ) in plasma before a next dose is administered, of less than 8, (e.g., less than 7, 6.5, 6, 5.5, 5, 4.5, 4, 3.5, 3, 2.5, 2 or lesser).
   23. The composition of any of embodiments 17-22, wherein, the amount, of ethanol is: sufficiently low that, upon injection, the pharmaceutical product provides a low initial release of setmelanotide; setmelanotide as sole active pharmaceutical ingredient formulated for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11).
   24. The composition of embodiment 23, wherein the low initial release is measured by the partial area under the drug concentration curve during the first hours (e.g., first 6 hours) after dosing, which is less than 10% or lesser, relative to the area under the drug concentration time curve of a 7 day, steady state dosing interval.
   25. The composition of embodiment 23, wherein the low initial release is measured by the partial area under the drug concentration curve during the first hours (e.g., first 12 hours) after dosing, which is less than 10-20% or lesser, relative to the area under the drug concentration time curve of a 7 day, steady state dosing interval.
   26. The composition of embodiment 23, wherein the low initial release is measured by the partial area under the drug concentration curve during the first hours (e.g., first 24 hours) after dosing, which is less than 20-30%, or lesser, relative to the area under the drug concentration time curve of a 7 day, steady state dosing interval.
   27. The compositions of embodiment 19, wherein the device can be a single use device, or a multi-use device.
   28. The composition of embodiment 19, wherein the device is chosen from: a manual syringe, (e.g., a syringe comprising a needle (e.g., a needle with a suitable diameter, e.g., a 27 Gauge needle), or an auto-injector (e.g., a spring-loaded syringe, or a pen injector).
   29. The composition of any one of the preceding embodiments, wherein the polar solvent, e.g., buffer, comprises citrate buffer, optionally wherein the pH of the buffer is 6.4.
   30. The composition of any one of the preceding embodiments, wherein the polar solvent, e.g., buffer, comprises citrate acid monohydrate.
   31. The composition of any one of the preceding embodiments, wherein the polar solvent, e.g., buffer, comprises an additional component, e.g., an antioxidant, or a chemical or physical stabilizing agent.
   32. The composition of embodiment 31, wherein the antioxidant is EDTA.
   33. The composition of any one of the preceding embodiments, wherein the polar solvent, e.g., buffer, comprises citric acid monohydrate, disodium EDTA, and water.
   34. The composition of any one of the preceding embodiments, wherein setmelanotide; setmelanotide as sole active pharmaceutical ingredient formulated for injection; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, is present as a chloride salt.
   35. The composition of any one of the preceding embodiments, wherein the pharmaceutical product optionally comprises an anti-microbial or microbial-static agent, e.g., bacteriostatic agent or preservative.
   36. The composition of any one of the preceding embodiments, wherein the ratio, by weight, of a:b is 70:30 to 40:60.
   37. The composition of any one of the preceding embodiments, wherein the ratio, by weight, of a:b is 70:30, 65:45, 60:40, 55:45, 50:50, 45:55 or 40:60
   38. The composition of any one of the preceding embodiments, wherein component a, is 20-80%, 30-70%, 33-60%, or 38-43% by weight of the total weight of components a, b, and c solution.
   39. The composition of any one of the preceding embodiments, wherein component b, is 20-80%, 30-70%, 33-60%, or 38-43% by weight of the total weight of components a, b, and c solution
   40. The composition of any one of the preceding embodiments, wherein component c, e.g., ethanol, is 0.5-50%, 2-30%, or 5-20% by weight of the total weight of components a, b, and c solution.
   41. The composition of any one of the preceding embodiments, wherein,
the neutral diacyl lipid comprises glycerol dioleate;
the phospholipid comprises phosphatidylcholine;
the alcohol comprises ethanol; and
the polar solvent, e.g., buffer, comprises a citrate buffer.
   42. The composition of any one of the preceding embodiments, wherein,
the neutral diacyl lipid comprises glycerol dioleate;
the phospholipid comprises soybean phosphatidylcholine;
the alcohol comprises ethanol; and
the polar solvent, e.g., buffer, comprises a citrate buffer at pH 6.4 comprising EDTA.
   43. The composition of any one of the preceding embodiments, comprising, per one milliliter of composition:
420+/−20% mg glycerol dioleate (GDO);
420+/−20% mg soybean phosphatidylcholine;
105+/−20% mg ethanol;
20+/−20% mg citrate buffer; and
30+/−20% mg setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   44. The composition of any one of the preceding embodiments, comprising, per one milliliter of composition:
420+/−10% mg glycerol dioleate (GDO);
420+/−10% mg soybean phosphatidylcholine;
105+/−10% mg ethanol;
20+/−10% mg citrate buffer; and
30+/−10% mg setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   45. The composition of any one of the preceding embodiments, comprising, per one milliliter of composition:
420+/−5% mg glycerol dioleate (GDO);
420+/−5% mg soybean phosphatidylcholine;
105+/−5% mg ethanol;
20+/−5% mg citrate buffer; and
30+/−5% mg setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   46. The composition of any one of the preceding embodiments, comprising, per one milliliter of composition:
420+/−2% mg glycerol dioleate (GDO);
420+/−2% mg soybean phosphatidylcholine;
105+/−2% mg ethanol;
20+/−2% mg citrate buffer; and
30+/−2% mg setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   47. The composition of any one of the preceding embodiments, comprising, per one milliliter of composition
419.8 mg glycerol dioleate (GDO);
419.8 mg soybean phosphatidylcholine;
105 mg ethanol;
20 mg citrate buffer; and
30 mg setmelanotide.
   48. The composition of any one of the preceding embodiments, comprising:
neutral diacyl lipid of component a at 20-80%, 30-70%, 33-60%, or 38-43% by weight, of components a, b, and c; components a, b, c, and d; or components a, b, c, d, and e of the composition;
phospholipid of component b at 20-80%, 30-70%, 33-60%, or 38-43% by weight, of components a, b, and c; components a, b, c, and d; or components a, b, c, d, and e of the composition;
alcohol of component c at 0.1-35%, 5-20%, 8-15%, or 9-11% by weight, of components a, b, and c; components a, b, c, and d; or components a, b, c, d, and e of the composition;
polar solvent of component d, e.g., buffer, at 0.5-10%, 1-5%, or 1-3% by weight, of components a, b, and c; components a, b, c, and d; or components a, b, c, d, and e of the composition; and
setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, at 0.1-10%, 0.2-8%, 0.5-6%, 1-4% by weight, of components a, b, and c; components a, b, c, and d; or components a, b, c, d, and e of the composition.
   49. The composition of any one of the preceding embodiments, comprising:
neutral diacyl lipid at 42%+/−10, 42%+1-5, 42%+/−2, or 42%+/−1 by weight, of components a, b, and c; components a, b, c, and d; or components a, b, c, d, and e of the composition;
phospholipid at 42%+/−10, 42%+1-5, 42%+/−2, or 42%+/−1 by weight, of components a, b, and c; components a, b, c, and d; or components a, b, c, d, and e of the composition;
alcohol at 10%+/−8, 10%+/−6, 10%+1-5, or 10%+/−1 by weight, of components a, b, and c; components a, b, c, and d; or components a, b, c, d, and e of the composition;
polar solvent, e.g., buffer, at 2%+/−1, 2%+1-0.5, 2%+/−0.25, or 2%+/−0.1 by weight, of components a, b, and c; components a, b, c, and d; or components a, b, c, d, and e of the composition; and
setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) at 3%+1-1.5, 3%+/−1, or 3%+1-0.5, by weight, of components a, b, and c; components a, b, c, and d; or components a, b, c, d, and e of the composition.
   50. The composition of any one of the preceding embodiments, which, upon contact with an aqueous environment, e.g., injection, e.g., subcutaneous injection, into a subject, forms or is capable of forming, at least one liquid crystalline structure.
   51. The composition of any one of the preceding embodiments, wherein the viscosity is:
(i) low enough to be comfortably delivered with a device, e.g., a syringe with a narrow needle, e.g., a 27 Gauge needle;
(ii) low enough that, upon injection, the initial burst, e.g., initial release, of setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), from the pharmaceutical product after subcutaneous injection gives a ratio of maximum concentration (Cₘₐₓ) in plasma to minimum concentration (Cₘᵢₙ) in plasma before a next dose is administered, of less than 8, (e.g., less than 7, 6.5, 6, 5.5, 5, 4.5, 4, 3.5, 3, 2.5, 2 or lesser); or
(iii) low enough that, upon injection, the pharmaceutical product provides a low initial release of setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11).
   52. The composition of any one of the preceding embodiments, wherein when injected into a subject:
i) the initial burst, e.g., initial release, of setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), from the pharmaceutical product after subcutaneous injection gives a ratio of maximum concentration (Cₘₐₓ) in plasma to minimum concentration (Cₘᵢₙ) in plasma before a next dose is administered, of less than 8, (e.g., less than 7, 6.5, 6, 5.5, 5, 4.5, 4, 3.5, 3, 2.5, 2 or lesser); or
ii) the pharmaceutical product provides a low initial release of setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11).
   53. The composition of embodiments 51 or 52, wherein the low initial release is measured by the partial area under the drug concentration curve during the first hours (e.g., first 6 hours) after dosing, which is less than 10% or lesser, relative to the area under the drug concentration time curve of a 7 day, steady state dosing interval.
   54. The composition of embodiments 51 or 52, wherein the low initial release is measured by the partial area under the drug concentration curve during the first hours (e.g., first 12 hours) after dosing, which is less than 10-20% or lesser, relative to the area under the drug concentration time curve of a 7 day, steady state dosing interval.
   55. The composition of embodiments 51 or 52, wherein the low initial release is measured by the partial area under the drug concentration curve during the first hours (e.g., first 24 hours) after dosing, which is less than 20-30%, or lesser, relative to the area under the drug concentration time curve of a 7 day, steady state dosing interval.
   56. The composition of any one of the preceding embodiments, comprising setmelanotide.
   57. The composition of any one of embodiments 1-55, comprising setmelanotide as sole active pharmaceutical ingredient formulated for injection.
   58. The composition of any one of embodiments 1-55, comprising setmelanotide as the sole active ingredient.
   59. The composition of any one of embodiments 1-55, comprising setmelanotide as the active pharmaceutical ingredient.
   60. The composition of any one of embodiments 1-55, comprising setmelanotide as the active pharmaceutical ingredient for injection.
   61. The composition of any one of embodiments 1-55, comprising a MC4RA_{P} (e.g, as the sole active ingredient, as the sole active ingredient for injection, as the active pharmaceutical ingredient, or as the active pharmaceutical ingredient for injection).
   62. The composition of any of embodiments 1-55, comprising BIM-22511.
   63. The composition of any of embodiments 1-55, comprising BIM-22287.
   64. The composition of any of embodiments 1-55, comprising BIM-22512.
   65. The composition of any of embodiments 1-55, comprising 001152C.
   66. The composition of any of embodiments 1-55, comprising 001543C.
   66a. The composition of any of embodiments 1-55, comprising 001003C.
   66b. The composition of any of embodiments 1-55, comprising 001574C.
   66c. The composition of any of embodiments 1-55, comprising 001555C.
   66d. The composition of any of embodiments 1-55, comprising 001554C.
   66e. The composition of any of embodiments 1-55, comprising 001556C.
   66f. The composition of any of embodiments 1-55, comprising 001358C.
   66g. The composition of any of embodiments 1-55, comprising 001576C.
   66h. The composition of any of embodiments 1-55, comprising 001364C.
   66i. The composition of any of embodiments 1-55, comprising 001258C.
   66j. The composition of any of embodiments 1-55, comprising MC4R-11.
   67. A unit dosage form comprising the composition of any of embodiments 1-76.
   68. The unit dosage form of embodiment 67, comprising at least 2, 1.8, 1.6, 1.4, 1.2, 1, 0.8, 0.6, 0.4, or 0.2 mL of the composition.
   69. The unit dosage form of any of embodiments 67-68, disposed in a liquid tight enclosure, e.g., a vial or a cartridge.
   70. The unit dosage form of any of embodiments 67-69 disposed in an injection device, e.g., a single use device, or a multi-use device.
   71. The unit dosage form of embodiment 70, wherein the device is chosen from: a manual syringe, (e.g., a syringe comprising a needle (e.g., a needle with a suitable diameter, e.g., a 27 Gauge needle), or an auto-injector (e.g., a spring-loaded syringe, or a pen injector).
   72. The unit dosage form of any of embodiments 67-71, containing 10-70; 20-60, 25-50; 25-40; 25-35 mg of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   73. The unit dosage form of any of embodiments 67-71, containing 50+/−20%; 40+/−20%; or 30+/−20%, mg of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   74. The unit dosage form of any of embodiments 67-71, containing 50+/−10%; 40+/−10%; or 30+/−10%, mg of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   75. The unit dosage form of any of embodiments 67-71, containing 50+/−5%; 40+/−5%; or 30+/−5%, mg of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   76. The unit dosage form of any of embodiments 67-71, containing 40, 35 or 30, mg of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   77. The unit dosage of any of embodiments 67-76, wherein the composition is suitable for injection, e.g., subcutaneous injection or intramuscular injection.
   78. The unit dosage form of any of embodiments 67-77, wherein the viscosity is:
i) low enough to be comfortably delivered with a device, e.g., a syringe with a narrow needle, e.g., a 27 Gauge needle;
ii) low enough that, upon injection, the initial burst, e.g., initial release, of setmelanotide; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), from the pharmaceutical product after subcutaneous injection gives a ratio of maximum concentration (Cₘₐₓ) in plasma to minimum concentration (Cₘᵢₙ) in plasma before a next dose is administered, of less than 8, (e.g., less than 7, 6.5, 6, 5.5, 5, 4.5, 4, 3.5, 3, 2.5, 2 or lesser); or
iii) low enough that, upon injection, the pharmaceutical product provides a low initial release of setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11).
   79. The unit dosage form of any of embodiments 67-77, wherein when injected into a subject:
i) the initial burst, e.g., initial release, of setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), from the pharmaceutical product after subcutaneous injection gives a ratio of maximum concentration (Cₘₐₓ) in plasma to minimum concentration (Cₘᵢₙ) in plasma before a next dose is administered, of less than 8, (e.g., less than 7, 6.5, 6, 5.5, 5, 4.5, 4, 3.5, 3, 2.5, 2 or lesser); or
ii) the pharmaceutical product provides a low initial release of setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11).
   80. The unit dosage form of embodiments 78 or 79, wherein the low initial release is measured by the partial area under the drug concentration curve during the first hours (e.g., first 6 hours) after dosing, which is less than 10% or lesser, relative to the area under the drug concentration time curve of a 7 day, steady state dosing interval.
   81. The unit dosage form of embodiments 78 or 79, wherein the low initial release is measured by the partial area under the drug concentration curve during the first hours (e.g., first 12 hours) after dosing, which is less than 10-20% or lesser, relative to the area under the drug concentration time curve of a 7 day, steady state dosing interval.
   82. The unit dosage form of embodiments 78 or 79, wherein the low initial release is measured by the partial area under the drug concentration curve during the first hours (e.g., first 24 hours) after dosing, which is less than 20-30%, or lesser, relative to the area under the drug concentration time curve of a 7 day, steady state dosing interval.
   83. A method of making a preparation or composition, e.g., a pharmaceutical composition, comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, e.g., a preparation or composition having a controlled level of EtOH, comprising:
i) providing a mixture of component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), a first amount of EtOH and one or more of components:
   a) a neutral diacyl lipid and/or a tocopherol;
   b) a phospholipid;
   c) an alcohol; and
   d) a polar solvent, e.g., a buffer; and
ii) adding a second amount of EtOH,
thereby making a preparation or composition, e.g., a pharmaceutical composition, comprising a component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   84. The method of embodiment 83, wherein providing comprises forming the mixture.
   85. The method of any of embodiments 83-84, wherein addition of the second amount of EtOH results in an amount of EtOH that meets a reference value, e.g., falls within a range of values, e.g., falls within a range defined by a lower and an upper value, e.g., an upper and lower value for weight % EtOH, e.g., weight % of 10-0.5.
   86. The method of embodiment 85, wherein the reference value is a value within the range of 5 to 20; 8.5 to 12.5; and 9 to 11 weight % EtOH, e.g., weight % of 10.
   87. The method of embodiment 85, wherein the reference value is a value within the range of 9 to 11 weight % EtOH, e.g., weight % of 10.
   88. The method of any of embodiments 83-87, comprising determining the amount of EtOH in the mixture by suitable analytical determination.
   89. The method of embodiment 88, comprising, responsive to the determination, selecting an amount of EtOH to add to the mixture, e.g., an amount that will achieve the reference value.
   90. The method of embodiment 89, comprising adding the selected amount of EtOH to the mixture to form an EtOH-replenished mixture.
   91. The method of embodiment 90, wherein the selected amount is added to the mixture as a single aliquot or as a plurality of aliquots of the same, or different, amounts.
   92. The method of any of embodiments 83-91, wherein less than 48 hours, less than 24 hours, or less than 4 hours elapses between forming the mixture and determining the amount of EtOH in the mixture, selecting an amount of EtOH to add, or adding the second amount.
   93. The method of any of embodiments 83-91 comprising providing the amount of the components added to the mixture.
   94. The method of embodiment 93, wherein providing comprises weighing the amount of components added to the mixture.
   95. The method of any of embodiments 83-94, comprising providing a value for the amount of EtOH in the mixture and determining the amount of EtOH that needs to be added to meet the reference for amount of EtOH.
   96. The method of any of embodiments 83-95, wherein after forming the mixture, EtOH is lost from the mixture, e.g., by evaporation, e.g., evaporation into the headspace of a container, e.g., a mixing vessel.
   97. The method of any of embodiments 83-96, wherein after the second amount is added to the mixture, determining the amount of EtOH in the EtOH-replenished mixture.
   98. The method of embodiment 97, comprising, responsive to the determination, selecting an amount of EtOH to add to the EtOH-replenished mixture.
   99. The method of embodiment 98 comprising adding the selected amount of EtOH to the EtOH-replenished mixture to form a twice-replenished mixture.
   100. The method of any of embodiments 83-99, further comprising providing a second a preparation or composition, e.g., a pharmaceutical product, comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   101. The method of any of embodiments 83-100, comprising:
making a mixture comprising component (e) comprising setmelanotide, EtOH, a neutral diacyl lipid and/or a tocopherol and a phospholipid,
wherein one or both of neutral diacyl lipid and/or a tocopherol and phospholipid, are added after setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) and the EtOH are combined,
thereby making component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection composition or preparation, e.g., a pharmaceutical product.
   102. The method of any of embodiments 83-100, comprising:
making a mixture comprising component (e) comprising setmelanotide or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), water or a polar solvent, e.g., a buffer, a neutral diacyl lipid and/or a tocopherol and a phospholipid,
wherein one or both of neutral diacyl lipid and/or a tocopherol and phospholipid, are added after component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) and water or the polar solvent, e.g., the buffer, are combined,
thereby making a component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection composition or preparation, e.g., a pharmaceutical product.
   103. The method of any of embodiments 83-102, wherein the order of formation of the mixture is:
i) component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) is contacted with EtOH (and optionally water or a polar solvent, e.g., a buffer);
ii) the phospholipid is added to the mixture resulting from i); and
iii) the neutral diacyl lipid and/or a tocopherol is added to the mixture resulting from ii).
   104. The method of embodiment 83, wherein the preparation or composition comprises setmelanotide.
   105. The method of embodiment 83, wherein the preparation or composition comprises setmelanotide as sole active pharmaceutical ingredient formulated for injection, setmelanotide as the sole active ingredient; setmelanotide as the active pharmaceutical ingredient; or setmelanotide as the active pharmaceutical ingredient for injection.
   106. The method of embodiment 83, wherein the preparation or composition comprises an MC4RA_{P}, e.g., as the sole active ingredient, as the sole active ingredient for injection, as the active pharmaceutical ingredient, or as the active pharmaceutical ingredient for injection.
   107. The method of embodiment 83, wherein the preparation or composition comprises BIM-22511.
   108. The method of embodiment 83, wherein the preparation or composition comprises BIM-22287.
   109. The method of embodiment 83, wherein the preparation or composition comprises BIM-22512.
   110. The method of embodiment 83, wherein the preparation or composition comprises 001152C.
   111. The method of embodiment 83, wherein the preparation or composition comprises 001543C.
   112. The method of embodiment 83, wherein the preparation or composition comprises 001003C.
   113. The method of embodiment 83, wherein the preparation or composition comprises 001574C.
   114. The method of embodiment 83, wherein the preparation or composition comprises 001555C.
   115. The method of embodiment 83, wherein the preparation or composition comprises 001554C.
   116. The method of embodiment 83, wherein the preparation or composition comprises 001556C.
   117. The method of embodiment 83, wherein the preparation or composition comprises 001358C.
   118. The method of embodiment 83, wherein the preparation or composition comprises 001576C.
   119. The method of embodiment 83, wherein the preparation or composition comprises 001364C.
   120. The method of embodiment 83, wherein the preparation or composition comprises 001258C.
   121. The method of embodiment 83, wherein the preparation or composition comprises MC4R-11.
   122. A method of making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a, component b, component d, and a predetermined amount of alcohol, comprising
combining (in any order) component (e) comprising setmelanotide; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), component a, component b, component d and alcohol, to mixture, and
comparing a value for alcohol content in the mixture with a reference value for alcohol content,
thereby making a formulation of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a, component b, component d, and a predetermined amount of alcohol.
   123. The method of embodiment 122, comprising: responsive to the value or comparison, increasing or decreasing the amount of alcohol in the mixture to provide a formulation having a predetermined amount of alcohol.
   124. The method of embodiment 123, comprising adding an addition amount of alcohol to the mixture.
   125. The method of embodiment 124, wherein the addition amount of alcohol is greater than the predetermined amount of alcohol.
   126. The method of embodiment 122, wherein the pharmaceutical product comprises setmelanotide.
   127. The method of embodiment 122, wherein the pharmaceutical product comprises setmelanotide as sole active pharmaceutical ingredient formulated for injection, setmelanotide as the sole active ingredient; setmelanotide as the active pharmaceutical ingredient, or setmelanotide as the active pharmaceutical ingredient for injection.
   128. The method of embodiment 122, wherein the pharmaceutical product comprises an MC4RA_{P}, e.g., as the sole active ingredient, as the sole active ingredient for injection, as the active pharmaceutical ingredient, or as the active pharmaceutical ingredient for injection
   129. The method of embodiment 122, wherein the pharmaceutical product comprises BIM-22511.
   130. The method of embodiment 122, wherein the pharmaceutical product comprises BIM-22287.
   131. The method of embodiment 122, wherein the pharmaceutical product comprises BIM-22512.
   132. The method of embodiment 122, wherein the pharmaceutical product comprises 001152C.
   133. The method of embodiment 122, wherein the pharmaceutical product comprises 001543C.
   134. The method of embodiment 122, wherein the pharmaceutical product comprises 001003C.
   135. The method of embodiment 122, wherein the pharmaceutical product comprises 001574C.
   136. The method of embodiment 122, wherein the pharmaceutical product comprises 001555C.
   137. The method of embodiment 122, wherein the pharmaceutical product comprises 001554C.
   138. The method of embodiment 122, wherein the pharmaceutical product comprises 001556C.
   139 The method of embodiment 122, wherein the pharmaceutical product comprises 001358C.
   140. The method of embodiment 122, wherein the pharmaceutical product comprises 001576C.
   141. The method of embodiment 122, wherein the pharmaceutical product comprises 001364C.
   142. The method of embodiment 122, wherein the pharmaceutical product comprises 001258C.
   143. The method of embodiment 122, wherein the pharmaceutical product comprises MC4R-11.
   144. A method of making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a (e.g., GDO), component b (e.g., soybean PC), component d (e.g., a polar solvent, e.g., a buffer, e.g., a citrate buffer at pH 6.4), and a predetermined amount of alcohol, comprising:
combining (in any order) component (e) comprising setmelanotide; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), component a (e.g., GDO), component b (e.g., soybean PC), component d (e.g., a polar solvent, e.g., a buffer, e.g., a citrate buffer at pH 6.4) and alcohol, to mixture, and
comparing a value for alcohol content in the mixture with a reference value for alcohol content,
thereby making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a (e.g., GDO), component b (e.g., soybean PC), component d (e.g., a polar solvent, e.g., a buffer, e.g., a citrate buffer at pH 6.4), and a predetermined amount of alcohol.
   145. The method of embodiment 144, comprising adding an addition amount of alcohol to the mixture.
   146. The method of embodiment 145, wherein the addition amount of alcohol is greater than the predetermined amount of alcohol.
   147. The method of any of embodiments 83-146, wherein, the predetermined amount is 5-20, 10-20, 15-20, 5-15, 5-10, 5-15, or 10-15% by weight.
   148. The method of any of embodiments 83-146, wherein, the predetermined amount is 10+/−5% by weight.
   149. The method of any of embodiments 83-146, wherein, the predetermined amount is 10+/−4% by weight.
   150. The method of any of embodiments 83-146, wherein, the predetermined amount is 10+/−3% by weight.
   151. The method of any of embodiments 83-146, wherein, the predetermined amount is 10+/−2% by weight.
   152. The method of any of embodiments 83-146, wherein, the predetermined amount is 10+/−1% by weight.
   153. The method of any of embodiments 83-146, wherein, the predetermined amount is 10+/−0.5% by weight.
   154. The method of any of embodiments 83-153, wherein the method comprises producing a plurality of batches of the formulation.
   155. The method of embodiment 154 wherein, each batch of the plurality of batches has an alcohol content within 2, 1, or 0.5% by weight of the other batch(es) in the plurality.
   156. The method of embodiment 154-155, wherein each batch of the plurality of batches has an alcohol content within 2, 1, or 0.5% by weight of a reference value.
   157. The method of embodiment 156, wherein, the reference value is a value within the range of:
5-20, 10-20, 15-20, 5-15, 5-10, 5-15, or 10-15% by weight;
10+/−5% by weight;
10+/−4% by weight;
10+/−3% by weight;
10+/−2% by weight;
10+/−1% by weight; or
10+/−0.5% by weight.
   158. The method of embodiments 155 or 156 wherein, the reference value is a value within the range of 10+/−2% by weight and each batch of the plurality of batches has an alcohol content within 1, or 0.5% by weight of the reference value.
   159. The method of any of embodiments 155 or 156 wherein, the reference value is 10% by weight and each batch of the plurality of batches has an alcohol content within 0.5% by weight of the reference value.
   160. The method of any of embodiments 154-159, wherein each batch of the plurality of batches has an alcohol content that is sufficiently great that the composition has a viscosity low enough to be comfortably delivered with a device, e.g., a syringe with a narrow needle, e.g., a 27 Gauge needle.
   161. The method of any of embodiments 154-159, wherein each batch of the plurality of batches, has an alcohol content that is sufficiently low that, upon injection, the initial burst of drug, e.g., initial release, after subcutaneous injection gives a ratio of maximum concentration (Cₘₐₓ) in plasma to minimum concentration (Cₘᵢₙ) in plasma before a next dose is administered, of less than 8, (e.g., less than 7, 6.5, 6, 5.5, 5, 4.5, 4, 3.5, 3, 2.5, 2 or lesser).
   162. The method of any of embodiments 154-159, wherein each batch of the plurality of batches, has an alcohol content that is sufficiently low that, upon injection, the pharmaceutical product provides a low initial release of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   163. The method of embodiment 162, wherein the low initial release is measured by the partial area under the drug concentration curve during the first hours (e.g., first 6 hours) after dosing, which is less than 10% or lesser, relative to the area under the drug concentration time curve of a 7 day, steady state dosing interval.
   164. The method of embodiment 162, wherein the low initial release is measured by the partial area under the drug concentration curve during the first hours (e.g., first 12 hours) after dosing, which is less than 10-20% or lesser, relative to the area under the drug concentration time curve of a 7 day, steady state dosing interval.
   165. The method of embodiment 162, wherein the low initial release is measured by the partial area under the drug concentration curve during the first hours (e.g., first 24 hours) after dosing, which is less than 20-30%, or lesser, relative to the area under the drug concentration time curve of a 7 day, steady state dosing interval.
   166. The method of embodiment 160, wherein the device can be a single use device, or a multi-use device.
   167. The method of embodiment 160 or 166 wherein the device is chosen from: a manual syringe, (e.g., a syringe comprising a needle (e.g., a needle with a suitable diameter, e.g., a 27 Gauge needle), or an auto-injector (e.g., a spring-loaded syringe, or a pen injector).
   168. The method of any of embodiments 122-153, wherein, the predetermined amount of alcohol is added as a single aliquot.
   169. The method of any of embodiments 122-153, wherein the predetermined amount of alcohol is added as a plurality of aliquots.
   170. The method of any of embodiments 122-153, or 168-169, wherein at least a portion of the process after addition of alcohol is performed in a closed vessel.
   171. The method of any of embodiments 122-153, or 168-170, wherein the process after addition of alcohol is performed in a closed vessel.
   172. The method of any of embodiments 122-153, or 168-170, wherein, at least a portion of the process prior to addition of alcohol is performed in a closed vessel.
   173. The method of any of embodiments 122-153, or 168-170, wherein the process prior to addition of alcohol is performed in a closed vessel.
   174. The method of embodiment 144, wherein the pharmaceutical product comprises setmelanotide.
   175. The method of embodiment 144, wherein the pharmaceutical product comprises setmelanotide as sole active pharmaceutical ingredient formulated for injection, setmelanotide as the sole active ingredient; setmelanotide as the active pharmaceutical ingredient, or setmelanotide as the active pharmaceutical ingredient for injection.
   176. The method of embodiment 144, wherein the pharmaceutical product comprises an MC4RA_{P}, e.g., as the sole active ingredient, as the sole active ingredient for injection, as the active pharmaceutical ingredient, or as the active pharmaceutical ingredient for injection.
   177. The method of embodiment 144, wherein the pharmaceutical product comprises BIM-22511.
   178. The method of embodiment 144, wherein the pharmaceutical product comprises BIM-22287.
   179. The method of embodiment 144, wherein the pharmaceutical product comprises BIM-22512.
   180. The method of embodiment 144, wherein the pharmaceutical product comprises 001152C.
   181. The method of embodiment 144, wherein the pharmaceutical product comprises 001543C.
   182. The method of embodiment 144, wherein the pharmaceutical product comprises 001003C.
   183. The method of embodiment 144, wherein the pharmaceutical product comprises 001574C.
   184. The method of embodiment 144, wherein the pharmaceutical product comprises 001555C.
   185. The method of embodiment 144, wherein the pharmaceutical product comprises 001554C.
   186. The method of embodiment 144, wherein the pharmaceutical product comprises 001556C.
   187 The method of embodiment 144, wherein the pharmaceutical product comprises 001358C.
   188. The method of embodiment 144, wherein the pharmaceutical product comprises 001576C.
   189. The method of embodiment 144, wherein the pharmaceutical product comprises 001364C.
   190. The method of embodiment 144, wherein the pharmaceutical product comprises 001258C.
   191. The method of embodiment 144, wherein the pharmaceutical product comprises MC4R-11.
   192. A method of making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection comprising:
(i) providing a mixture comprising component (e) comprising setmelanotide; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), and alcohol (a component (e)-alcohol mixture), e.g., component (e) comprising setmelanotide; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), in contact with, e.g., dissolved or dispersed in an alcohol, e.g., ethanol; and
(ii) combining component (e) comprising setmelanotide; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11)-alcohol mixture with an amount of component a (e.g, GDO), component b (e.g., soybean PC), and component d (e.g., citrate buffer at pH 6.4), or all of components a, b, and d.
   193. The method of embodiment 192, wherein step (i), (ii), or (i) and (ii) are performed in a closed vessel.
   194. The method of embodiment 192, wherein the pharmaceutical product comprises setmelanotide.
   195. The method of embodiment 192, wherein the pharmaceutical product comprises setmelanotide as sole active pharmaceutical ingredient formulated for injection, setmelanotide as the sole active ingredient, setmelanotide as the active pharmaceutical ingredient, or setmelanotide as the active pharmaceutical ingredient for injection
   196. The method of embodiment 192, wherein the pharmaceutical product comprises an MC4RA_{P}, e.g., as the sole active ingredient, as the sole active ingredient for injection, as the active pharmaceutical ingredient, or as the active pharmaceutical ingredient for injection.
   197. The method of embodiment 192, wherein the pharmaceutical product comprises BIM-22511.
   198. The method of embodiment 192, wherein the pharmaceutical product comprises BIM-22287.
   199. The method of embodiment 192, wherein the pharmaceutical product comprises BIM-22512.
   200. The method of embodiment 192, wherein the pharmaceutical product comprises 001152C.
   201. The method of embodiment 192, wherein the pharmaceutical product comprises 001543C.
   202. The method of embodiment 192, wherein the pharmaceutical product comprises 001003C.
   203. The method of embodiment 192, wherein the pharmaceutical product comprises 001574C.
   204. The method of embodiment 192, wherein the pharmaceutical product comprises 001555C.
   205. The method of embodiment 192, wherein the pharmaceutical product comprises 001554C.
   206. The method of embodiment 192, wherein the pharmaceutical product comprises 001556C.
   207 The method of embodiment 192, wherein the pharmaceutical product comprises 001358C.
   208. The method of embodiment 192, wherein the pharmaceutical product comprises 001576C.
   209. The method of embodiment 192, wherein the pharmaceutical product comprises 001364C.
   210. The method of embodiment 192, wherein the pharmaceutical product comprises 001258C.
   211. The method of embodiment 192, wherein the pharmaceutical product comprises MC4R-11.
   212. A method of making a preparation comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, or evaluating a candidate preparation, e.g., for a quality control or release specification, comprising:
providing a value for the amount of EtOH in a candidate preparation of component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11); and
comparing the value with a reference value for amount of EtOH;
thereby making a preparation of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   213. The method of embodiment 212, further comprising, responsive to the comparison, selecting the candidate preparation of component (e) comprising setmelanotide or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11).
   214. The method of any of embodiments 212-213, wherein the reference value comprises a range defined by a lower and an upper value, e.g., an upper and lower value for weight % EtOH, e.g., weight % of 10.
   215. The method of embodiment 214, wherein meeting the reference value comprises falling within the range.
   216. The method of embodiments 214, wherein the reference value is a value within the range of 5 to 20; 8.5 to 12.5; and 9 to 11 weight % EtOH, e.g., weight % of 10.
   217. The method of embodiment 214, wherein the reference value is a value within the range of 9 to 11 weight % EtOH, e.g., weight % of 10.
   218. The method of any of embodiments 212-217, comprising providing a value for the amount of EtOH in a second candidate preparation of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection; and comparing the value with a reference value for amount of EtOH.
   219. The method of any of embodiments 212-217, comprising
providing a value for the amount of EtOH in N candidate preparations of component (e) comprising setmelanotide; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), wherein N is equal to or greater than 1, 2, 3, 4, 5, 6, 7, 8, 9, 10, 50, 100, or 1,000; and
comparing the value with a reference value for amount of EtOH.
   220. The method of any of embodiments 218-219, wherein the amount of EtOH in each of the plurality of preparations is within 5, 2, 1, or 0.5% of one another.
   221. The method of any of embodiments 218-219, wherein the amount of EtOH in each of the plurality of preparations is within 2% of one another.
   222. The method of any of embodiments 218-219, wherein the amount of EtOH in each of the plurality of preparations is within 0.5% of one another.
   223. The method of any of embodiments 212-222, wherein a first preparation and a second preparation of the plurality are made within 10, 20, 30, 60, 180, or 365 days of one another.
   224. The method of any of embodiments 212-223 wherein a second preparation is made less than 10, 20, 30, 60, 180, or 365 days after the first preparation.
   225. The method of embodiment 212, wherein the preparation comprises setmelanotide.
   226. The method of embodiment 212, wherein the preparation comprises setmelanotide as sole active pharmaceutical ingredient formulated for injection, setmelanotide as the sole active ingredient; setmelanotide as the active pharmaceutical ingredient, or setmelanotide as the active pharmaceutical ingredient for injection.
   227. The method of embodiment 212, wherein the preparation comprises an MC4RA_{P}. e.g., as the sole active ingredient, as the sole active ingredient for injection, as the active pharmaceutical ingredient, or as the active pharmaceutical ingredient for injection.
   228. The method of embodiment 212, wherein the preparation comprises BIM-22511.
   229. The method of embodiment 212, wherein the preparation comprises BIM-22287.
   230. The method of embodiment 212, wherein the preparation comprises BIM-22512.
   231. The method of embodiment 212, wherein the preparation comprises 001152C.
   232. The method of embodiment 212, wherein the preparation comprises 001543C.
   233. The method of embodiment 212, wherein the preparation comprises 001003C.
   234. The method of embodiment 212, wherein the preparation comprises 001574C.
   235. The method of embodiment 212, wherein the preparation comprises 001555C.
   236. The method of embodiment 212, wherein the preparation comprises 001554C.
   237. The method of embodiment 212, wherein the preparation comprises 001556C.
   238. The method of embodiment 212, wherein the preparation comprises 001358C.
   239. The method of embodiment 212, wherein the preparation comprises 001576C.
   240. The method of embodiment 212, wherein the preparation comprises 001364C.
   241. The method of embodiment 212, wherein the preparation comprises 001258C.
   242. The method of embodiment 212, wherein the preparation comprises MC4R-11.
   243. A method of making a component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection composition or preparation, e.g., a pharmaceutical product, comprising:
making a mixture comprising component (e) comprising setmelanotide or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), EtOH, a neutral diacyl lipid and/or a tocopherol and a phospholipid,
wherein one or both of neutral diacyl lipid and/or a tocopherol and phospholipid, are added after component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) and the EtOH are combined,
thereby making a component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection composition or preparation, e.g., a pharmaceutical product.
   244. The method of embodiment 243, wherein one or both of neutral diacyl lipid and/or a tocopherol and a phospholipid are added after at least 10%, 25%, 50%, 75%, or all of the component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) is allowed to go into solution.
   245. The method of any of embodiments 243-244, wherein the order of formation of the mixture is:
i) component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) is contacted with EtOH (and optionally water or buffer);
ii) phospholipid is added to the mixture resulting from i); and
iii) neutral diacyl lipid and/or a tocopherol is added to the mixture resulting from ii).
   246. The method of embodiment 243, wherein the composition or preparation comprises setmelanotide.
   247. The method of embodiment 243, wherein the composition or preparation comprises setmelanotide as sole active pharmaceutical ingredient formulated for injection, setmelanotide as the sole active ingredient; setmelanotide as the active pharmaceutical ingredient, or setmelanotide as the active pharmaceutical ingredient for injection.
   248. The method of embodiment 243, wherein the composition or preparation comprises an MC4RA_{P}, e.g., as the sole active ingredient, as the sole active ingredient for injection, as the active pharmaceutical ingredient, or as the active pharmaceutical ingredient for injection.
   249. The method of embodiment 243, wherein the composition or preparation comprises BIM-22511.
   250. The method of embodiment 243, wherein the composition or preparation comprises BIM-22287.
   251. The method of embodiment 243, wherein the composition or preparation comprises BIM-22512.
   252. The method of embodiment 243, wherein the composition or preparation comprises 001152C.
   253. The method of embodiment 243, wherein the composition or preparation comprises 001543C.
   254. The method of embodiment 243, wherein the composition or preparation comprises 001003C.
   255. The method of embodiment 243, wherein the composition or preparation comprises 001574C.
   256. The method of embodiment 243, wherein the composition or preparation comprises 001555C.
   257. The method of embodiment 243, wherein the composition or preparation comprises 001554C.
   258. The method of embodiment 243, wherein the composition or preparation comprises 001556C.
   259. The method of embodiment 243, wherein the composition or preparation comprises 001358C.
   260. The method of embodiment 243, wherein the composition or preparation comprises 001576C.
   261. The method of embodiment 243, wherein the composition or preparation comprises 001364C.
   262. The method of embodiment 243, wherein the composition or preparation comprises 001258C.
   263. The method of embodiment 243, wherein the composition or preparation comprises MC4R-11.
   264. A method of making a pharmaceutical product of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection comprising the following steps in order:
(i) providing a mixture comprising setmelanotide; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) and alcohol (component (e)-alcohol mixture), e.g., setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) in contact with, e.g., dissolved or dispersed in an alcohol, e.g., ethanol; and
(ii) combining the component (e)-alcohol mixture with an amount of component a (e.g., GDO), component b (e.g., soybean PC), and component d (e.g., citrate buffer at pH 6.4), or an amount of all of components a, b, and d;
   thereby making a pharmaceutical product of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, e.g., a pharmaceutical product of setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection comprising a predetermined amount of alcohol, e.g., 10 wt. % alcohol, e.g., ethanol.
      265. The method of embodiment 264, wherein the pharmaceutical product comprises setmelanotide.
      266. The method of embodiment 264, wherein the pharmaceutical product comprises setmelanotide as sole active pharmaceutical ingredient formulated for injection, setmelanotide as the sole active ingredient, setmelanotide as the active pharmaceutical ingredient, or setmelanotide as the active pharmaceutical ingredient for injection.
      267. The method of embodiment 264, wherein the pharmaceutical product comprises an MC4RA_{P}, e.g., as the sole active ingredient, as the sole active ingredient for injection, as the active pharmaceutical ingredient, or as the active pharmaceutical ingredient for injection.
      268. The method of embodiment 264, wherein the pharmaceutical product comprises BIM-22511.
      269. The method of embodiment 264, wherein the pharmaceutical product comprises BIM-22287.
      270. The method of embodiment 264, wherein the pharmaceutical product comprises BIM-22512.
      271. The method of embodiment 264, wherein the pharmaceutical product comprises 001152C.
      272. The method of embodiment 264, wherein the pharmaceutical product comprises 001543C.
      273. The method of embodiment 264, wherein the pharmaceutical product comprises 001003C.
      274. The method of embodiment 264, wherein the pharmaceutical product comprises 001574C.
      275. The method of embodiment 264, wherein the pharmaceutical product comprises 001555C.
      276. The method of embodiment 264, wherein the pharmaceutical product comprises 001554C.
      277. The method of embodiment 264, wherein the pharmaceutical product comprises 001556C.
      278 The method of embodiment 264, wherein the pharmaceutical product comprises 001358C.
      279. The method of embodiment 264, wherein the pharmaceutical product comprises 001576C.
      280. The method of embodiment 264, wherein the pharmaceutical product comprises 001364C.
      281. The method of embodiment 264, wherein the pharmaceutical product comprises 001258C.
      282. The method of embodiment 264, wherein the pharmaceutical product comprises MC4R-11.
      283. A method of making a pharmaceutical product of component (e) comprising setmelanotide comprising the following steps in order:
(i) providing a mixture comprising setmelanotide; and alcohol (a setmelanotide-alcohol mixture), e.g., setmelanotide, in contact with, e.g., dissolved or dispersed in, an alcohol, e.g., ethanol; and
(ii) combining the setmelanotide-alcohol mixture with an amount of component a (e.g., GDO), component b (e.g., soybean PC), and component d (e.g., citrate buffer at pH 6.4), or an amount of all of components a, b, and d;
thereby making a pharmaceutical product of component (e) comprising setmelanotide; e.g., a pharmaceutical product comprising setmelanotide comprising a predetermined amount of alcohol, e.g., 10 wt. % alcohol, e.g., ethanol.
   284. A method of making a pharmaceutical product of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection comprising the following steps in order:
(i) providing a mixture of component (e) comprising setmelanotide or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), alcohol and component d, e.g., a polar solvent, e.g., a buffer, (a component (e)-alcohol-buffer mixture), e.g., component (e) comprising setmelanotide or an MC4RA_{P} (e.g., BIM-22511, BIM-22287, BIM-22512, 001152C, 001543C, 001003C, 001574C, 001555C. 001554C. 001556C, 001358C, 001576C, 001364C, 001258C, or MC4R-11) in contact with, e.g., dissolved or dispersed in, an alcohol, e.g., ethanol, and a buffer; and
(ii) combining the component (e)-alcohol-buffer mixture with an amount of one or more of components a, e.g., GDO, and b, e.g., soybean PC;
thereby making a pharmaceutical product of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, e.g., a pharmaceutical product of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection comprising a predetermined amount of alcohol, e.g., 10 wt. % alcohol, e.g., ethanol.
   285. The method of embodiment 284, wherein the pharmaceutical product comprises setmelanotide.
   286. The method of embodiment 284, wherein the pharmaceutical product comprises setmelanotide as sole active pharmaceutical ingredient formulated for injection, setmelanotide as the sole active ingredient; setmelanotide as the active pharmaceutical ingredient, or setmelanotide as the active pharmaceutical ingredient for injection.
   287. The method of embodiment 284, wherein the pharmaceutical product comprises an MC4RA_{P}, e.g., as the sole active ingredient, as the sole active ingredient for injection, as the active pharmaceutical ingredient, or as the active pharmaceutical ingredient for injection.
   288. The method of embodiment 284, wherein the pharmaceutical product comprises BIM-22511.
   289. The method of embodiment 284, wherein the pharmaceutical product comprises BIM-22287.
   290. The method of embodiment 284, wherein the pharmaceutical product comprises BIM-22512.
   291. The method of embodiment 284, wherein the pharmaceutical product comprises 001152C.
   292. The method of embodiment 284, wherein the pharmaceutical product comprises 001543C.
   293. The method of embodiment 284, wherein the pharmaceutical product comprises 001003C.
   294. The method of embodiment 284, wherein the pharmaceutical product comprises 001574C.
   295. The method of embodiment 284, wherein the pharmaceutical product comprises 001555C.
   296. The method of embodiment 284, wherein the pharmaceutical product comprises 001554C.
   297. The method of embodiment 284, wherein the pharmaceutical product comprises 001556C.
   298 The method of embodiment 284, wherein the pharmaceutical product comprises 001358C.
   299. The method of embodiment 284, wherein the pharmaceutical product comprises 001576C.
   300. The method of embodiment 284, wherein the pharmaceutical product comprises 001364C.
   301. The method of embodiment 284, wherein the pharmaceutical product comprises 001258C.
   302. The method of embodiment 284, wherein the pharmaceutical product comprises MC4R-11.
   303. A method of making a pharmaceutical product of component (e) comprising setmelanotide comprising the following steps in order:
(i) providing a mixture comprising component (e) comprising setmelanotide; alcohol and component d, e.g., a polar solvent, e.g., a buffer, e.g., a citrate buffer at pH 6.4 (a setmelanotide-alcohol-buffer mixture), e.g., setmelanotide in contact with, e.g., dissolved or dispersed in, an alcohol, e.g., ethanol, and a citrate buffer at pH 6.4; and
(ii) combining the component (e)-alcohol-buffer mixture with an amount of component a (e.g., GDO), and component b (e.g., soybean PC), or an amount of all of components a and b;
   thereby making a pharmaceutical product of component (e) comprising setmelanotide; e.g., a pharmaceutical product of component (e) comprising setmelanotide comprising a predetermined amount of alcohol, e.g., 10 wt. % alcohol, e.g., ethanol.
      304. A method of making a pharmaceutical product component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a, component b, component d, and a predetermined amount of alcohol, comprising
combining, in a specified order, component (e) comprising setmelanotide or an MC4RA_{P}(e.g., BIM-22511. BIM-22287, BIM-22512, 001152C, 001543C, 001003C, 001574C, 001555C, 001554C, 001556C, 001358C, 001576C, 001364C, 001258C, or MC4R-11), component a, component b, component d and an addition amount of alcohol, wherein the addition amount of alcohol results in a predetermined amount of alcohol in the pharmaceutical product, and wherein the specified order comprises the following steps in order:
   (i) providing a mixture comprising component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC-4R-11) and an addition amount of alcohol (a component (e)-alcohol mixture), e.g., component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) in contact with, e.g., dissolved or dispersed in, an alcohol, e.g., ethanol; and
   (ii) combining the component (e)-alcohol mixture with an amount of components a, b, and d or all of components a, b, and d;
wherein (i), (ii) or (i) and (ii) are performed in a closed vessel, thereby making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a, component b, component d, and a predetermined amount of alcohol, e.g., 10 wt. % alcohol, e.g., ethanol.
   305. The method of embodiment 304, wherein the pharmaceutical product comprises setmelanotide.
   306. The method of embodiment 304, wherein the pharmaceutical product comprises setmelanotide as sole active pharmaceutical ingredient formulated for injection, setmelanotide as the sole active ingredient, setmelanotide as the active pharmaceutical ingredient, or setmelanotide as the active pharmaceutical ingredient for injection.
   307. The method of embodiment 304, wherein the pharmaceutical product comprises an MC4RA_{P}, e.g., as the sole active ingredient, as the sole active ingredient for injection, as the active pharmaceutical ingredient, or as the active pharmaceutical ingredient for injection.
   308. The method of embodiment 304, wherein the pharmaceutical product comprises BIM-22511.
   309. The method of embodiment 304, wherein the pharmaceutical product comprises BIM-22287.
   310. The method of embodiment 304, wherein the pharmaceutical product comprises BIM-2512.
   311. The method of embodiment 304, wherein the pharmaceutical product comprises 001152C.
   312. The method of embodiment 304, wherein the pharmaceutical product comprises 001543C.
   313. The method of embodiment 304, wherein the pharmaceutical product comprises 001003C.
   314. The method of embodiment 304, wherein the pharmaceutical product comprises 001574C.
   315. The method of embodiment 304, wherein the pharmaceutical product comprises 001555C.
   316. The method of embodiment 304, wherein the pharmaceutical product comprises 001554C.
   317. The method of embodiment 304, wherein the pharmaceutical product comprises 001556C.
   318 The method of embodiment 304, wherein the pharmaceutical product comprises 001358C.
   319. The method of embodiment 304, wherein the pharmaceutical product comprises 001576C.
   320. The method of embodiment 304, wherein the pharmaceutical product comprises 001364C.
   321. The method of embodiment 304, wherein the pharmaceutical product comprises 001258C.
   322. The method of embodiment 304, wherein the pharmaceutical product comprises MC4R-11.
   323. A method of making a pharmaceutical product comprising setmelanotide; component a, component b, component d, and a predetermined amount of alcohol, comprising
combining, in a specified order, setmelanotide, component a, component b, component d and an addition amount of alcohol, wherein the addition amount of alcohol results in a predetermined amount of alcohol in the pharmaceutical product, and wherein the specified order comprises the following steps in order:
(i) providing a mixture comprising setmelanotide and an addition amount of alcohol (a setmelanotide-alcohol mixture), e.g., setmelanotide in contact with, e.g., dissolved or dispersed in, an alcohol, e.g., ethanol; and
(ii) combining the setmelanotide-alcohol mixture with an amount of components a, b, and d or all of components a, b, and d;
wherein (i), (ii) or (i) and (ii) are performed in a closed vessel, thereby making a pharmaceutical product comprising setmelanotide, component a, component b, component d, and a predetermined amount of alcohol, e.g., 10 wt. % alcohol, e.g., ethanol.
   324. A method of making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a (e.g., GDO), component b (e.g., soybean PC), component d (e.g., a polar solvent, e.g., a buffer, e.g., a citrate buffer at pH 6.4), and a predetermined amount of alcohol, comprising
combining, in a specified order, component (e) comprising setmelanotide or an MC4RA_{P} (e.g., BIM-22511. BIM-22287, BIM-22512, 001152C, 001543C, 001003C, 001574C, 001555C, 001554C, 001556C, 001358C, 001576C, 001364C, 001258C, or MC4R-11), component a (e.g., GDO), component b (e.g., soybean PC), component d (e.g., a polar solvent, e.g., a buffer, e.g., a citrate buffer at pH 6.4), and an addition amount of alcohol, wherein the addition amount of alcohol results in a predetermined amount of alcohol in the pharmaceutical product, wherein the specified order comprises the following steps in order:
(i) providing a mixture comprising component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) and an addition amount of alcohol (a component (e)-alcohol mixture), e.g., component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) in contact with, e.g., dissolved or dispersed in, an alcohol, e.g., ethanol; and
(ii) combining the component (e)-alcohol mixture with an amount of component a (e.g., GDO), component b (e.g., soybean PC), and component d (e.g., citrate buffer at pH 6.4), or all of components a, b, and d;
wherein (i), (ii) or (i) and (ii) are performed in a closed vessel, thereby making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a (e.g., GDO), component b (e.g., soybean PC), component d (e.g., a polar solvent, e.g., a buffer, e.g., a citrate buffer at pH 6.4), and a predetermined amount of alcohol, e.g., 10 wt. % alcohol, e.g., ethanol.
   325. The method of embodiment 324, wherein the pharmaceutical product comprises setmelanotide.
   326. The method of embodiment 324, wherein the pharmaceutical product comprises setmelanotide as sole active pharmaceutical ingredient formulated for injection, setmelanotide as the sole active ingredient, setmelanotide as the active pharmaceutical ingredient, or setmelanotide as the active pharmaceutical ingredient for injection.
   327. The method of embodiment 324, wherein the pharmaceutical product comprises an MC4RA_{P}, e.g., as the sole active ingredient, as the sole active ingredient for injection, as the active pharmaceutical ingredient, or as the active pharmaceutical ingredient for injection.
   328. The method of embodiment 324, wherein the pharmaceutical product comprises BIM-22511.
   329. The method of embodiment 324, wherein the pharmaceutical product comprises BIM-22287.
   330. The method of embodiment 324, wherein the pharmaceutical product comprises BIM-22512.
   331. The method of embodiment 324, wherein the pharmaceutical product comprises 001152C.
   332. The method of embodiment 324, wherein the pharmaceutical product comprises 001543C.
   333. The method of embodiment 324, wherein the pharmaceutical product comprises 001003C.
   334. The method of embodiment 324, wherein the pharmaceutical product comprises 001574C.
   335. The method of embodiment 324, wherein the pharmaceutical product comprises 001555C.
   336. The method of embodiment 324, wherein the pharmaceutical product comprises 001554C.
   337. The method of embodiment 324, wherein the pharmaceutical product comprises 001556C.
   338. The method of embodiment 324, wherein the pharmaceutical product comprises 001358C.
   339. The method of embodiment 324, wherein the pharmaceutical product comprises 001576C.
   340. The method of embodiment 324, wherein the pharmaceutical product comprises 001364C.
   341. The method of embodiment 324, wherein the pharmaceutical product comprises 001258C.
   342. The method of embodiment 324, wherein the pharmaceutical product comprises MC4R-11.
   343. A method of making a pharmaceutical product comprising setmelanotide, component a (e.g., GDO), component b (e.g., soybean PC), component d (e.g., a polar solvent, e.g., a buffer, e.g., a citrate buffer at pH 6.4), and a predetermined amount of alcohol, comprising
combining, in a specified order, setmelanotide; component a (e.g., GDO), component b (e.g., soybean PC), component d (e.g., a polar solvent, e.g., a buffer, e.g., a citrate buffer at pH 6.4), and an addition amount of alcohol, wherein the addition amount of alcohol results in a predetermined amount of alcohol in the pharmaceutical product, wherein the specified order comprises the following steps in order:
(i) providing a mixture comprising setmelanotide and an addition amount of alcohol (a setmelanotide-alcohol mixture), e.g., setmelanotide in contact with, e.g., dissolved or dispersed in, an alcohol, e.g., ethanol; and
(ii) combining the setmelanotide-alcohol mixture with an amount of component a (e.g., GDO), component b (e.g., soybean PC), and component d (e.g., citrate buffer at pH 6.4), or all of components a, b, and d;
wherein (i), (ii) or (i) and (ii) are performed in a closed vessel, thereby making a pharmaceutical product comprising setmelanotide, component a (e.g., GDO), component b (e.g., soybean PC), component d (e.g., a polar solvent, e.g., a buffer, e.g., a citrate buffer at pH 6.4), and a predetermined amount of alcohol, e.g., 10 wt. % alcohol, e.g., ethanol.
   344. A method of making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a, component b, component d, and a predetermined amount of alcohol, comprising
combining, in a specified order, component (e) comprising setmelanotide or an MC4RA_{P} (e.g., BIM-22511, BM-22287, BIM-22512, 001152C, 001543C, 001003C, 001574C, 001555C, 001554C, 001556C, 001358C, 001576C, 001364C, 001258C, or MC4R-11), component a, component b, component d and an addition amount of alcohol, wherein the addition amount of alcohol results in a predetermined amount of alcohol in the pharmaceutical product, wherein the specified order comprises the following steps in order:
(i) providing a mixture comprising component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), an addition amount of alcohol and component d, e.g., a polar solvent, e.g., a buffer, e.g., a citrate buffer at pH 6.4 (a component (e)-buffer mixture), e.g., component (e) comprising setmelanotide or an MC4RA_{P} (e.g., BIM-22511. BIM-22287, BIM-22512, 001152C, 001543C, 001003C, 001574C, 001555C, 001554C, 001556C, 001358C, 001576C, 001364C, 001258C, or MC4R-11 in contact with, e.g., dissolved or dispersed in, an alcohol, e.g., ethanol and a citrate buffer at pH 6.4; and
(ii) combining the component (e)-alcohol-buffer mixture with an amount of components a and b or all of components a and b;
wherein (i), (ii) or (i) and (ii) are performed in a closed vessel, thereby making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a, component b, component d, and a predetermined amount of alcohol, e.g., 10 wt. % alcohol, e.g., ethanol.
   345. The method of embodiment 344, wherein the pharmaceutical product comprises setmelanotide.
   346. The method of embodiment 344, wherein the pharmaceutical product comprises setmelanotide as sole active pharmaceutical ingredient formulated for injection, setmelanotide as the sole active ingredient, setmelanotide as the active pharmaceutical ingredient, or setmelanotide as the active pharmaceutical ingredient for injection.
   347. The method of embodiment 344, wherein the pharmaceutical product comprises an MC4RA_{P}, e.g., as the sole active ingredient, as the sole active ingredient for injection, as the active pharmaceutical ingredient, or as the active pharmaceutical ingredient for injection
   348. The method of embodiment 344, wherein the pharmaceutical product comprises BIM-22511.
   349. The method of embodiment 344, wherein the pharmaceutical product comprises BIM-22287.
   350. The method of embodiment 344, wherein the pharmaceutical product comprises BIM-22512.
   351. The method of embodiment 344, wherein the pharmaceutical product comprises 001152C.
   352. The method of embodiment 344, wherein the pharmaceutical product comprises 001543C.
   353. The method of embodiment 344, wherein the pharmaceutical product comprises 001003C.
   354. The method of embodiment 344, wherein the pharmaceutical product comprises 001574C.
   355. The method of embodiment 344, wherein the pharmaceutical product comprises 001555C.
   356. The method of embodiment 344, wherein the pharmaceutical product comprises 001554C.
   357. The method of embodiment 344, wherein the pharmaceutical product comprises 001556C.
   358. The method of embodiment 344, wherein the pharmaceutical product comprises 001358C.
   359. The method of embodiment 344, wherein the pharmaceutical product comprises 001576C.
   360. The method of embodiment 344, wherein the pharmaceutical product comprises 001364C.
   361. The method of embodiment 344, wherein the pharmaceutical product comprises 001258C.
   362. The method of embodiment 344, wherein the pharmaceutical product comprises MC4R-11.
   363. A method of making a pharmaceutical product comprising setmelanotide, component a, component b, component d, and a predetermined amount of alcohol, comprising
combining, in a specified order, the setmelanotide, component a, component b, component d and an addition amount of alcohol, wherein the addition amount of alcohol results in a predetermined amount of alcohol in the pharmaceutical product, wherein the specified order comprises the following steps in order:
   (i) providing a mixture comprising a setmelanotide, an addition amount of alcohol and component d, e.g., a polar solvent, e.g., a buffer, e.g., a citrate buffer at pH 6.4 (a setmelanotide-alcohol-buffer mixture), e.g., setmelanotide in contact with, e.g., dissolved or dispersed in, an alcohol, e.g., ethanol and a citrate buffer at pH 6.4; and
   (ii) combining setmelanotide-alcohol-buffer mixture with an amount of components a and b or all of components a and b;
wherein (i), (ii) or (i) and (ii) are performed in a closed vessel, thereby making a pharmaceutical product comprising setmelanotide, component a, component b, component d, and a predetermined amount of alcohol, e.g., 10 wt. % alcohol, e.g., ethanol.
   364. A method of making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a (e.g., GDO), component b (e.g., soybean PC), component d (e.g., a polar solvent, e.g., a buffer, e.g., a citrate buffer at pH 6.4), and a predetermined amount of alcohol, comprising
combining, in a specified order, component (e) comprising setmelanotide or an MC4RA_{f}(e.g., BIM-22511. BIM-22287, BIM-22512, 001152C, 001543C, 001003C, 001574C, 001555C, 001554C, 001556C, 001358C, 001576C, 001364C, 001258C, or MC4R-11), component a, component b, component d and an addition amount of alcohol, wherein the addition amount of alcohol results in a predetermined amount of alcohol in the pharmaceutical product, wherein the specified order comprises the following steps in order:
(i) providing a mixture comprising component (e) comprising setmelanotide or an MC4RA_{P} (e.g, BIM-22511, BIM-22287, BIM-22512, 001152C, 001543C, 001003C, 001574C, 001555C, 001554C, 001556C, 001358C, 001576C, 001364C, 001258C. or MC4R-11), an addition amount of alcohol and component d, e.g., a polar solvent, e.g., a buffer, e.g., a citrate buffer at pH 6.4 (a component (e)-alcohol-buffer mixture), e.g., component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11) in contact with, e.g., dissolved or dispersed in, an alcohol, e.g., ethanol and a citrate buffer at pH 6.4; and
(ii) combining the component (e)-alcohol-buffer mixture with an amount of component a (e.g., GDO), and component b (e.g., soybean PC) or all of components a and b;
wherein (i), (ii) or (i) and (ii) are performed in a closed vessel, thereby making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, component a (e.g., GDO), component b (e.g., soybean PC), component d (e.g., a polar solvent, e.g., a buffer, e.g., a citrate buffer at pH 6.4), and a predetermined amount of alcohol, e.g., 10 wt. % alcohol, e.g., ethanol.
   365. The method of embodiment 364, wherein the pharmaceutical product comprises setmelanotide.
   366. The method of embodiment 364, wherein the pharmaceutical product comprises setmelanotide as sole active pharmaceutical ingredient formulated for injection, setmelanotide as the sole active ingredient, setmelanotide as the active pharmaceutical ingredient, or setmelanotide as the active pharmaceutical ingredient for injection.
   367. The method of embodiment 364, wherein the pharmaceutical product comprises an MC4RA_{P}, as the sole active ingredient, as the sole active ingredient for injection, as the active pharmaceutical ingredient, or as the active pharmaceutical ingredient for injection.
   368. The method of embodiment 364, wherein the pharmaceutical product comprises BIM-22511.
   369. The method of embodiment 364, wherein the pharmaceutical product comprises BIM-22287.
   370. The method of embodiment 364, wherein the pharmaceutical product comprises BIM-22512.
   371. The method of embodiment 364, wherein the pharmaceutical product comprises 001152C.
   372. The method of embodiment 364, wherein the pharmaceutical product comprises 001543C.
   373. The method of embodiment 364, wherein the pharmaceutical product comprises 001003C.
   374. The method of embodiment 364, wherein the pharmaceutical product comprises 001574C.
   375. The method of embodiment 364, wherein the pharmaceutical product comprises 001555C.
   376. The method of embodiment 364, wherein the pharmaceutical product comprises 001554C.
   377. The method of embodiment 364, wherein the pharmaceutical product comprises 001556C.
   378 The method of embodiment 364, wherein the pharmaceutical product comprises 001358C.
   379. The method of embodiment 364, wherein the pharmaceutical product comprises 001576C.
   380. The method of embodiment 364, wherein the pharmaceutical product comprises 001364C.
   381. The method of embodiment 364, wherein the pharmaceutical product comprises 001258C.
   382. The method of embodiment 364, wherein the pharmaceutical product comprises MC4R-11.
   383. A method of making a pharmaceutical product comprising setmelanotide, component a (e.g., GDO), component b (e.g., soybean PC), component d (e.g., a polar solvent, e.g., a buffer, e.g., a citrate buffer at pH 6.4), and a predetermined amount of alcohol, comprising
combining, in a specified order, the setmelanotide, component a, component b, component d and an addition amount of alcohol, wherein the addition amount of alcohol results in a predetermined amount of alcohol in the pharmaceutical product, wherein the specified order comprises the following steps in order:
   (i) providing a mixture comprising the setmelanotide, an addition amount of alcohol and component d, e.g., a polar solvent, e.g., a buffer, e.g., a citrate buffer at pH 6.4 (a setmelanotide-alcohol-buffer mixture), e.g., setmelanotide in contact with, e.g., dissolved or dispersed in, an alcohol, e.g., ethanol and a citrate buffer at pH 6.4; and
   (ii) combining the setmelanotide-alcohol-buffer mixture with an amount of component a (e.g., GDO), and component b (e.g., soybean PC) or all of components a and b;
wherein (i), (ii) or (i) and (ii) are performed in a closed vessel, thereby making a pharmaceutical product comprising setmelanotide, component a (e.g., GDO), component b (e.g., soybean PC), component d (e.g., a polar solvent, e.g., a buffer, e.g., a citrate buffer at pH 6.4), and a predetermined amount of alcohol, e.g., 10 wt. % alcohol, e.g., ethanol.
   384. A method of making a pharmaceutical product comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection comprising the following steps in order:
i) combining an amount of one or more of (e.g., all of) components a, b, c and d;
ii) providing to this mixture component (e) comprising setmelanotide or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11); and
iii) mixing the mixture of (i) and (ii) for a specified amount of time to dissolve or disperse component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11);
thereby making a pharmaceutical product of component (e) setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, e.g., a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, comprising a predetermined amount of alcohol, e.g., 10 wt. % alcohol, e.g., ethanol.
   385. The method of embodiment 384, wherein the pharmaceutical product comprises setmelanotide.
   386. The method of embodiment 384, wherein the pharmaceutical product comprises setmelanotide as sole active pharmaceutical ingredient formulated for injection, setmelanotide as the sole active ingredient, setmelanotide as the active pharmaceutical ingredient, or setmelanotide as the active pharmaceutical ingredient for injection.
   387. The method of embodiment 384, wherein the pharmaceutical product comprises an MC4RA_{P} e.g., as the sole active ingredient, as the sole active ingredient for injection, as the active pharmaceutical ingredient, or as the active pharmaceutical ingredient for injection
   388. The method of embodiment 384, wherein the pharmaceutical product comprises BIM-22511.
   389. The method of embodiment 384, wherein the pharmaceutical product comprises BIM-22287.
   390. The method of embodiment 384, wherein the pharmaceutical product comprises BIM-22512.
   391. The method of embodiment 384, wherein the pharmaceutical product comprises 001152C.
   392. The method of embodiment 384, wherein the pharmaceutical product comprises 001543C.
   393. The method of embodiment 384, wherein the pharmaceutical product comprises 001003C.
   394. The method of embodiment 384, wherein the pharmaceutical product comprises 001574C.
   395. The method of embodiment 384, wherein the pharmaceutical product comprises 001555C.
   396. The method of embodiment 384, wherein the pharmaceutical product comprises 001554C.
   397. The method of embodiment 384, wherein the pharmaceutical product comprises 001556C.
   398. The method of embodiment 384, wherein the pharmaceutical product comprises 001358C.
   399. The method of embodiment 384, wherein the pharmaceutical product comprises 001576C.
   400. The method of embodiment 384, wherein the pharmaceutical product comprises 001364C.
   401. The method of embodiment 384, wherein the pharmaceutical product comprises 001258C.
   402. The method of embodiment 384, wherein the pharmaceutical product comprises MC4R-11.
   403. The method of any of embodiments 384-402, wherein the mixing is performed for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours.
   404. The method of any of embodiments 384-402, wherein the mixing is performed for 1-30 hours.
   405. The method of any of embodiments 384-402, wherein the mixing is performed for no more than 30, 40, or 50 hours.
   406. A method of making a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection comprising the following steps in order:
i) combining an amount of one or more of (e.g., all of) components a, b, d and component (e) comprising setmelanotide or an MC4RA_{P} (e.g., BIM-22511, 131M-22287, BIM-22512, 001152C, 001543C, 001003C, 001574C, 001555C, 001554C, 001556C, 001358C, 001576C, 001364C, 001258C, or MC4R-11);
ii) providing to this mixture a predetermined amount of component c; and
iii) mixing the mixture of (i) and (ii) for a specified amount of time to dissolve or disperse component (e) comprising setmelanotide or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11);
thereby making a pharmaceutical product of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, e.g., a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, comprising a predetermined amount of alcohol, e.g., 10 wt. % alcohol, e.g., ethanol.
   407. The method of embodiment 406, wherein the pharmaceutical product comprises setmelanotide.
   408. The method of embodiment 406, wherein the pharmaceutical product comprises setmelanotide as sole active pharmaceutical ingredient formulated for injection, setmelanotide as the sole active ingredient, setmelanotide as the active pharmaceutical ingredient, or setmelanotide as the active pharmaceutical ingredient for injection.
   409. The method of embodiment 406, wherein the pharmaceutical product comprises an MC4RA_{P}, e.g., as the sole active ingredient, as the sole active ingredient for injection, as the active pharmaceutical ingredient, or as the active pharmaceutical ingredient for injection
   410. The method of embodiment 406, wherein the pharmaceutical product comprises BIM-22511.
   411. The method of embodiment 406, wherein the pharmaceutical product comprises BIM-22287.
   412. The method of embodiment 406, wherein the pharmaceutical product comprises BIM-22512.
   413. The method of embodiment 406, wherein the pharmaceutical product comprises 001152C.
   414. The method of embodiment 406, wherein the pharmaceutical product comprises 001543C.
   415. The method of embodiment 406, wherein the pharmaceutical product comprises 001003C.
   416. The method of embodiment 406, wherein the pharmaceutical product comprises 001574C.
   417. The method of embodiment 406, wherein the pharmaceutical product comprises 001555C.
   418. The method of embodiment 406, wherein the pharmaceutical product comprises 001554C.
   419. The method of embodiment 406, wherein the pharmaceutical product comprises 001556C.
   420. The method of embodiment 406, wherein the pharmaceutical product comprises 001358C.
   421. The method of embodiment 406, wherein the pharmaceutical product comprises 001576C.
   422. The method of embodiment 406, wherein the pharmaceutical product comprises 001364C.
   423. The method of embodiment 406, wherein the pharmaceutical product comprises 001258C.
   424. The method of embodiment 406, wherein the pharmaceutical product comprises MC4R-11.
   425. The method of any of embodiments 406-424, wherein the mixing is performed for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours.
   426. The method of any of embodiments 406-424, wherein the mixing is performed for 1-30 hours.
   427. The method of any of embodiments 406-424, wherein the mixing is performed for no more than 30, 40, or 50 hours.
   428. A plurality of preparations, e.g., a first preparation, a second preparation, a third preparation or more, of a pharmaceutical product of component (e) setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, each preparation of the plurality having an amount of EtOH that falls within a predetermined range, e.g., a plurality of preparations made by a method of one or more of embodiments 65 to 427.
   429. The plurality of preparations of a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, of embodiment 428, wherein the amount of EtOH in each of the plurality of preparations is within 2, 1, or 0.5% by weight of one another.
   430. The plurality of preparations of a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, of embodiment 428, wherein the amount of EtOH in each of the plurality of preparations is within the range of 10%+1-5, 10%+/−4, 10%+/−3, 10%+/−2, or 10%+/−1 by weight of one another.
   431. The plurality of preparations of a pharmaceutical product of component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection of any of embodiments 428-430, wherein a first preparation and a second preparation of the plurality are made within 10, 20, 30, 60, 180, or 365 days of one another.
   432. The plurality of preparations of a pharmaceutical product comprising component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P}(e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection of embodiment 428-431, wherein a second preparation is made less than 10, 20, 30, 60, 180, or 365 days of the first preparation.
   433. The method of embodiment 428, wherein the plurality of preparations comprises setmelanotide.
   434. The method of embodiment 428, wherein the plurality of preparations comprises setmelanotide as sole active pharmaceutical ingredient formulated for injection, setmelanotide as the sole active ingredient; setmelanotide as the active pharmaceutical ingredient, or setmelanotide as the active pharmaceutical ingredient for injection.
   435. The method of embodiment 428, wherein the plurality of preparations comprises an MC4RA_{P}, e.g., as the active pharmaceutical ingredient, or as the active pharmaceutical ingredient for injection.
   436. The method of embodiment 428, wherein the plurality of preparations comprises BIM-22511.
   437. The method of embodiment 428, wherein the plurality of preparations comprises BIM-22287.
   438. The method of embodiment 428, wherein the plurality of preparations comprises BIM-22512.
   439. The method of embodiment 428, wherein the plurality of preparations comprises 001152C.
   440. The method of embodiment 428, wherein the plurality of preparations comprises 001543C.
   441. The method of embodiment 428, wherein the plurality of preparations comprises 001003C.
   442. The method of embodiment 428, wherein the plurality of preparations comprises 001574C.
   443. The method of embodiment 428, wherein the plurality of preparations comprises 001555C.
   444. The method of embodiment 428, wherein the plurality of preparations comprises 001554C.
   445. The method of embodiment 428, wherein the plurality of preparations comprises 001556C.
   446 The method of embodiment 428, wherein the plurality of preparations comprises 001358C.
   447. The method of embodiment 428, wherein the plurality of preparations comprises 001576C.
   448. The method of embodiment 428, wherein the plurality of preparations comprises 001364C.
   449. The method of embodiment 428, wherein the plurality of preparations comprises 001258C.
   450. The method of embodiment 428, wherein the plurality of preparations comprises MC4R-11.
   451. A method of providing component (e) comprising setmelanotide; setmelanotide as sole active pharmaceutical ingredient formulated for injection; setmelanotide as the sole active ingredient; setmelanotide as the active pharmaceutical ingredient; setmelanotide as the active pharmaceutical ingredient for injection; or an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection, to a subject, comprising administering to the subject, and effective amount of a pharmaceutical product of any of embodiments 1-450, or a pharmaceutical product made by one or more of the methods of embodiments 65-427.
   452. The method of embodiment 451, wherein the pharmaceutical product comprises setmelanotide.
   453. The method of embodiment 451, wherein the pharmaceutical product comprises setmelanotide as sole active pharmaceutical ingredient formulated for injection, setmelanotide as the sole active ingredient, setmelanotide as the active pharmaceutical ingredient, or setmelanotide as the active pharmaceutical ingredient for injection.
   454. The method of embodiment 451, wherein the pharmaceutical product comprises an MC4RA_{P} (e.g., BIM-22511, BIM-22287, BIM-22512, 001152C, 001543C, 001003C, 001574C, 001555C, 001554C, 001556C, 001358C, 001576C, 001364C, 001258C, or MC4R-11), as the sole active ingredient, as the sole active ingredient for injection, as the active pharmaceutical ingredient, or as the active pharmaceutical ingredient for injection.
   455. The method of embodiment 451, wherein the pharmaceutical product comprises BIM-22511.
   456. The method of embodiment 451, wherein the pharmaceutical product comprises BIM-22287.
   457. The method of embodiment 451, wherein the pharmaceutical product comprises BIM-22512.
   459. The method of embodiment 451, wherein the pharmaceutical product comprises 001152C.
   460. The method of embodiment 451, wherein the pharmaceutical product comprises 001543C.
   461. The method of embodiment 451, wherein the pharmaceutical product comprises 001003C.
   462. The method of embodiment 451, wherein the pharmaceutical product comprises 001574C.
   463. The method of embodiment 451, wherein the pharmaceutical product comprises 001555C.
   464. The method of embodiment 451, wherein the pharmaceutical product comprises 001554C.
   465. The method of embodiment 451, wherein the pharmaceutical product comprises 001556C.
   466. The method of embodiment 451, wherein the pharmaceutical product comprises 001358C.
   467. The method of embodiment 451, wherein the pharmaceutical product comprises 001576C.
   468. The method of embodiment 451, wherein the pharmaceutical product comprises 001364C.
   469. The method of embodiment 451, wherein the pharmaceutical product comprises 001258C.
   470. The method of embodiment 451, wherein the pharmaceutical product comprises MC4R-11.
   471. The method of embodiment 451, wherein the subject has, or is at risk of having a disorder responsive to modulation of melanocortin-4 receptor (MC4R).
   472. The method of embodiment 471, wherein the disorder is chosen from: type 1 diabetes, type 2 diabetes, obesity, insulin resistance, metabolic syndrome, male erectile dysfunction, female sexual disorder, non-alcoholic fatty liver disease, non-alcoholic steatohepatitis, disorders of substance abuse, including alcoholism, feeding disorders, cachexia, inflammation or anxiety, Prader-Willi Syndrome, Bardet-Biedl syndrome, and Alström syndrome.
   473. The method of embodiment 472, wherein the disorder is obesity.
   474. The method of embodiment 472, wherein the disorder is type 1 diabetes.
   475. The method of embodiment 472, wherein the disorder is type 2 diabetes.
   476. The method of embodiment 472, wherein the disorder is Prader-Willi Syndrome.
   477. The method of embodiment 472, wherein the disorder is Bardet-Biedl syndrome.
   478. The method of embodiment 472, wherein the disorder is Alström syndrome.

### EXAMPLES

### Example 1: Pharmacokinetics of Setmelanotide Formulations

Embodiments of sustained, e.g., extended, release of the pharmaceutical product, disclosed herein have desirable pharmacokinetic properties, e.g., a decreased Cₘₐₓ with no change or an increase in the Area Under the Curve (AUC).

### Methods

Eleven different formulations of the pharmaceutical product were made and assessed for their in vivo pharmacokinetics properties in Cynomolgus monkeys. Formulations 5A-5D are described in Table 2. Formulations 3A and 4A are described in Table 3. Formulations 1A-1E are described in Table 4.

For each formulation of the pharmaceutical product, six male Cynomolgus monkeys were dosed with a single SC injection of either 0.5 mg/kg (formulations 1A-1C, 3A-4A and 5A-5C) or 1.5 mg/kg (formulations 1D-1E, and 5D). Between nine and eleven serial blood samples (approximately 0.8 mL/sample) were drawn from each subject over a period of 48 to 240 hours. Plasma (containing 1% HALT™ protease inhibitor) was harvested, frozen and shipped to Worldwide Clinical Trials, Austin, Texas, for analysis. Monkey plasma samples were analyzed for the peptide. The method used in this study was validated for a range of 5.00 to 2000 ng/mL based on the analysis of 0.100 mL of plasma. Quantitation was performed using a weighted 1/×2 linear least squares regression analysis generated from calibration standards.

### Results

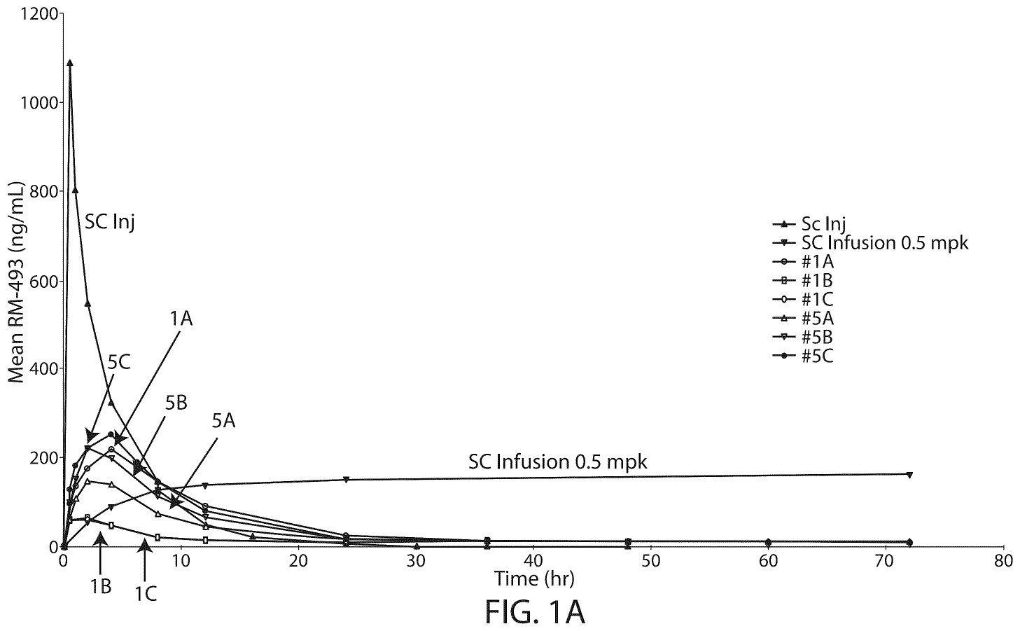

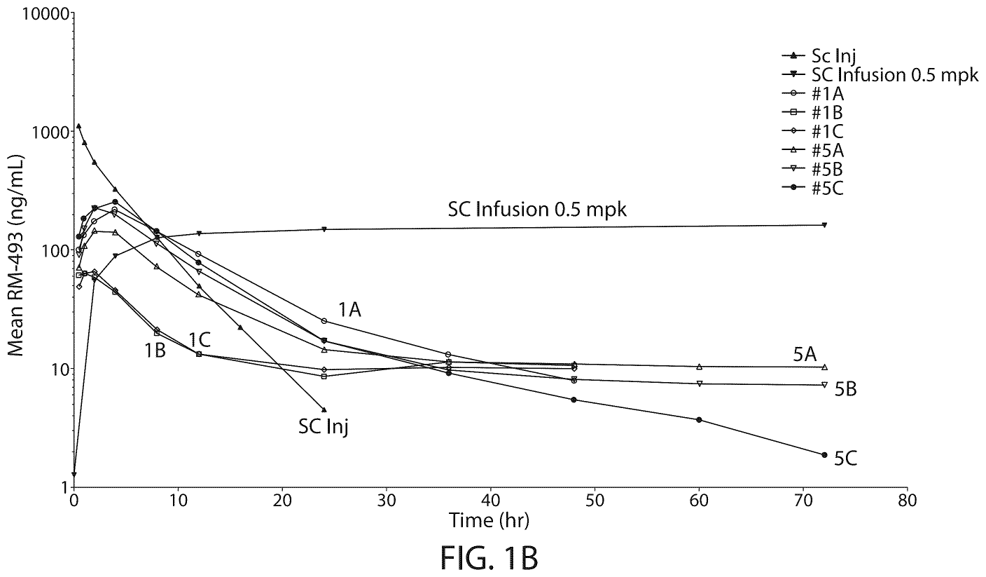

FIGS. 1A and 1B illustrate the mean concentration data from SC injection of pharmaceutical product 1A-1C, 5A-5C, and 3A/4A, and an SC infusion administration from a previous study. The SC infusion of the peptide formulation (3A and 4A) demonstrated a low Cₘₐₓ and a large AUC, while SC injection of the same pharmaceutical product showed a higher Cₘₐₓ and much smaller AUC.

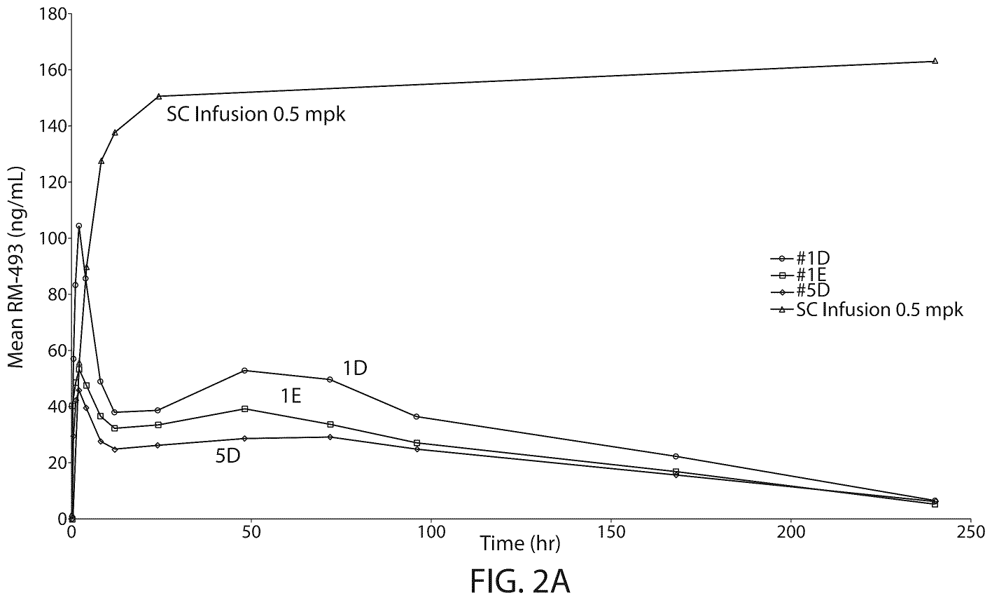

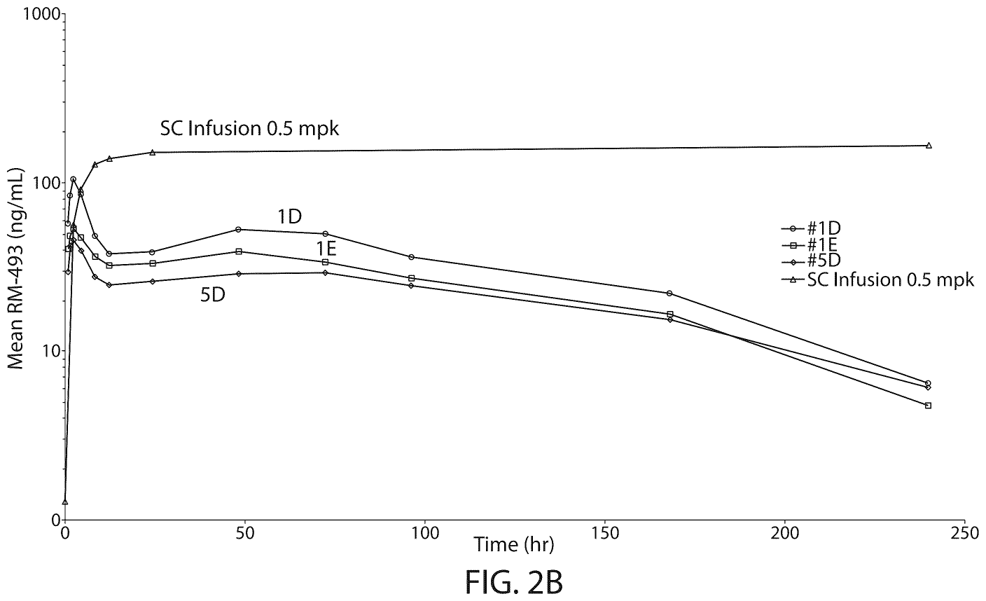

FIGS. 2A and 2B illustrate the mean concentration data from pharmaceutical products 1D-1E, 5D, and the dose-normalized SC infusion data from a previous study. As in FIGS. 2A and 2B, the SC infusion of the pharmaceutical product demonstrated a low Cₘₐₓ and a large AUC. Pharmaceutical product 1D, 1E and 5D had concentration-time profiles with an initial peak at 2 hours, a secondary peak or plateau between 48 and 96 hours, and then slowly decreasing between 96 and 240 hours. The data show that these pharmaceutical product dosages provide for an initial sustained burst of drug immediately after SC injection. The initial burst is followed by a period of continuous release and a subsequent 24 to 48 hour period when the pharmaceutical product release rate slightly increases.

TABLE 2
Formulations 5A-5D used in Example 1.

| PK | | Target Dose |
|---|---|---|
| Group | PK Treatment Description | (mg/kg) |
| 5A | 1.85/38.08/38.08/10/10/2 | 0.5 |
| | RYM/SPC/GDO/EtOH/PG/P80 | |
| 5B | 1.85/37.58/37.58/10/10/3 | 0.5 |
| | RYM/SPC/GDO/EtOH/PG/P80 | |
| 5C | 1.85/37.08/37.08/10/10/4 | 0.5 |
| | RYM/SPC/GDO/EtOH/PG/P80 | |
| 5D | 3.74/42.13/42.13/10/2 | 1.5 |
| | RYM/SPC/GDO/EtOH/Ca buf pH 6.4(EDTA) | |

TABLE 3
Formulations 3A and 4A used in Example 1.

| PK | | Target Dose |
|---|---|---|
| Group | PK Treatment Description | (mg/kg) |
| 3A | 10 mg/mL RM-493 | 0.5 |
| 4A | 10 mg/mL RM-493 | 0.5 |

TABLE 4
Formulations 1A-1E used in Example 1.

| PK | | Target Dose |
|---|---|---|
| Group | PK Treatment Description | (mg/kg) |
| 1A | 2/39/39/7.5/7.5/5 | 0.5 |
| | RYM/SPC/GDO/EtOH/PG/P80 | |
| 1B | 2.02/33.99/33.99/15/15 | 0.5 |
| | RYM/SPC/GDO/EtOH/WFI | |
| 1C | 2/39/39/10/10 | 0.5 |
| | RYM/SPC/GDO/EtOH/PG | |
| 1D | 3.95/40.53/40.53/7.5/7.5 | 1.5 |
| | RYM/SPC/GDO/EtOH/PG | |
| 1E | ;4.04/42.98/42.98/10 | 1.5 |
| | RYM/SPC/GDO/EtOH | |

RYM = Setmelanotide; SPC = Soybean phosphatidylcholine; GDO = glycerol dioleate; EtOH = Ethanol; PG = Propylene Glycol; P80 = Polysorbate 80; WFI = water for injection, contains 0.1 mg/ml of EDTA

### Example 2. Pharmacokinetics of Setmelanotide Formulations

In order to assess the pharmacokinetics of setmelanotide, a two part study was carried out comprising single dose administration (Part A) and multiple dose administration (Part B) with healthy obese subjects.

Part A was a rising-dose, placebo-controlled study including up to 3 sequential panels. For each panel, 10 normal obese subjects in good health between the ages of 21 and 54 were assigned to randomly-ordered treatments which included 8 active and 2 placebo treatments. Data acquired from Part A were used to determine the dose and dosing regimen for Part B. In Part B, 12 normal healthy obese patients were randomly assigned to receive either 6 active and 6 placebo treatments. Subjects received once weekly subcutaneous injection of the assigned formulation for 4 weeks. Patients in Part B were housed in the clinical unit for the 28 days.

Setmelanotide was formulated in a sterile liquid polar lipid phase consisting of soy phosphatidylcholine, glycerol dioleate, ethanol and citrate buffer. The concentration of each component in the formulation is summarized in Table 5. Without being bound by theory, the setmelanotide formulation behaves in a long-acting fashion, wherein when injected subcutaneously, aqueous body fluid is absorbed by the lipid phase to form a gel like depot consisting of liquid crystals formed in situ.

TABLE 5
Formulation components for PK study

| | | Quantity |
|---|---|---|
| Component | Function | (mg per vial) |
| Setmelanotide | Active pharmaceutical | 30.00 |
| (expressed as net peptide) | ingredient | |
| Citrate Buffer/pH 6.4 [a] | Co-solvent | 20.00 |
| Ethanol | Co-solvent | 105.00 |
| Soybean | Structure forming lipid | 419.80 |
| Phosphatidylcholine | | |
| Glycerol Dioleate | Structure forming lipid | 419.80 |
| Nitrogen | Sparging gas (process aid) | q.s. |

### Part A (Single-Dose Phase)

10 normal obese subjects enrolled for each dose level of Part A; 8 received active drug, and 2 received placebo. There were 3 dose levels used in the single dose phase which were administered according to the sequential, rising dose design. The single setmelanotide doses administered were in sequence: 2.5 mg, 10 mg, and 30 mg. Setmelanotide concentrations were analyzed and safety was assessed before the next higher dose level was started. The drug was administered subcutaneously on the morning of dosing. Blood samples were collected prior to dosing and at 0.5, 1, 2, 3, 4, 6, 8, 10, 12, 16, 24, 30, and 36 hours post-dose (in the clinic), then at approximately 48, 60, 72, 96, 120, 168, 240, and 336 hours post-dose as outpatients. Based on the PK analysis of the first dose, additional blood samples were included for the 30 mg dose at 21 (480 hours) and 28 (648 hours) days post-dose. The actual times of blood collection were recorded.

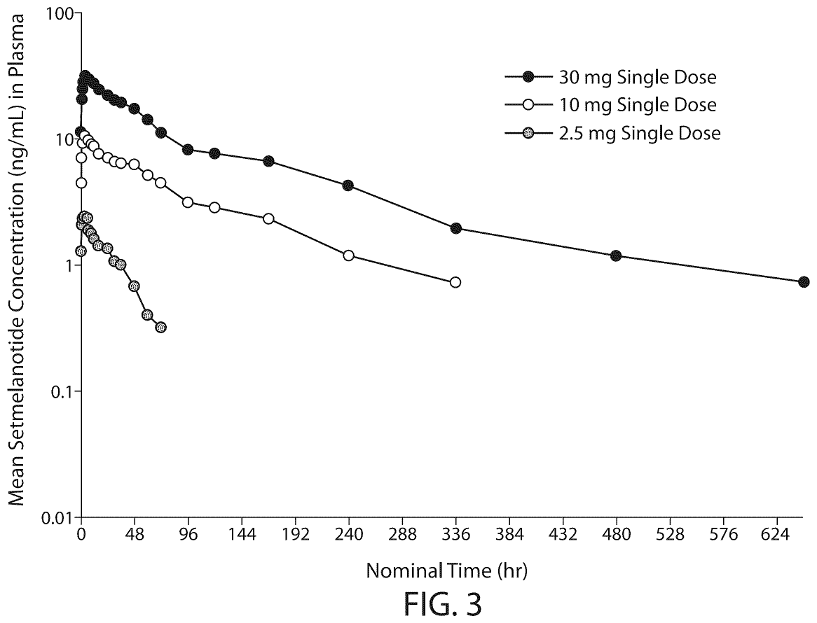

Mean setmelanotide concentration for each sample at all three dose levels were quantified and are presented in FIG. 3. Generally, setmelanotide appeared in the earliest plasma sample time (0.5 hr) and slowly rose to maximum concentrations about 4 hours after dosing. Setmelanotide was measurable in plasma for 2 weeks or more after dosing. From the semi-logarithmic plot, the terminal phases of the 10 mg and 30 mg doses appear to be linear and parallel. However, the mean profile for the 2.5 mg dose indicates that setmelanotide concentrations in the plasms were considerably lower compared to the other two treatments, and concentrations decreased to below quantifiable levels from 24 to 96 hours after dosing. All values for the 2.5 mg dose were reported as below quantifiable by 96 hr (4 days after dosing).

### Part B (Multiple-Dose Phase)

Twelve (12) obese subjects were enrolled in Part B; 6 received active drug, and 6 were given placebo. Based on results from Part A, setmelanotide 10 mg once weekly was selected as the dose for Part B. Subjects were housed in the clinical facility for the entire 4 weeks of study. Qualified subjects entered the clinical facility the evening before the first dose. Subjects assigned to active treatment received setmelanotide 10 mg subcutaneously in the morning on Day 1. Placebo subjects received the same volume of the placebo injection as the active treatment. Subjects were fasting from the evening before to 4 hours after dosing. Standard lowfat meals were served. Subjects were also dosed on Days 8, 15, and 22 with the treatment given on Day 1.

Blood samples were collected at a time before dosing and at 0.5, 1, 2, 3, 4, 6, 8, 10, 12, 16, 24, 30, 36, 48, 60, 72, 96, 120, 168, 240, and 336 hours during Week 1 and during Week 4. The actual sampling times were recorded. In addition, blood samples were collected at a time just before dosing for trough setmelanotide concentrations for evaluating the attainment of steady state.

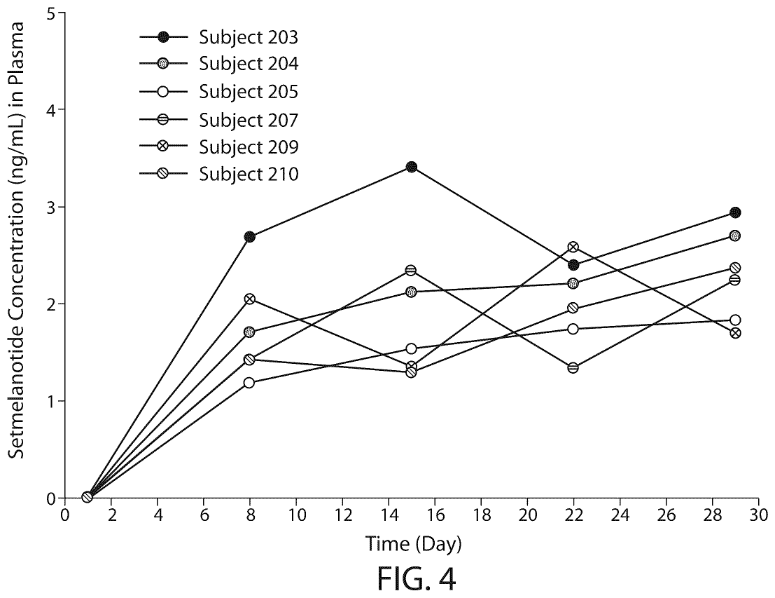

Trough setmelanotide concentrations for the six subjects during the multiple-dose phase of Part B are shown in FIG. 4. By visual inspection, it appeared that steady state was attained during Week 4 after 4 weekly doses of the test formulation.

### CONCLUSION

The PK objectives of this study were to evaluate the PK performance of an investigational setmelanotide injectable formulation designed for once weekly administration. The results of this study showed that for single dose administration, there was no early burst effect or dose dumping. Setmelanotide absorption started soon after injection with little to no lag-time. Mean Cₘₐₓ concentrations were 2.92, 11.5 and 34.1 ng/mL and median tₘₐₓ values were 3.5, 4.0 and 6.0 hr for 2.5, 10 and 30 mg doses, respectively. Mean AUC_{∞} values were 92.9, 1142 and 3474 hr*ng/mL, respectively. For the 2.5 mg dose, setmelanotide concentrations were measurable for 24 to 72 hours after dosing. In contrast, the profiles for the 10 mg and 30 mg doses were defined past 168 hours. The long half-life values for the 10 and 30 mg doses (mean values were 123 and 159 hr, respectively) indicate that absorption was still occurring during the week after dosing. Further, Cₘₐₓ was roughly proportional over the 2.5 to 30 mg dose range. AUC was proportional for the 10 mg and 30 mg doses. AUC_{∞} for the 2.5 mg dose was less than proportional because plasma concentrations were undetectable after 24 to 72 hours.

For multiple dose administration, providing setmelanotide as a 10 mg dose once weekly for 4 weeks produced a mean Cₘax of 11.4 mg/mL at a median tₘₐₓ of 4 hours. There was no lag in absorption, and setmelanotide concentration declined over the 1-week dosing interval to a mean concentration at 168 hours of 2.92 ng/mL. The mean AUC, for Week 4 was similar in magnitude to the mean AUC_{∞} for Week 1, and the accumulation index for setmelanotide was 1.46.

### Example 3. Stability Analysis of Setmelanotide Formulations

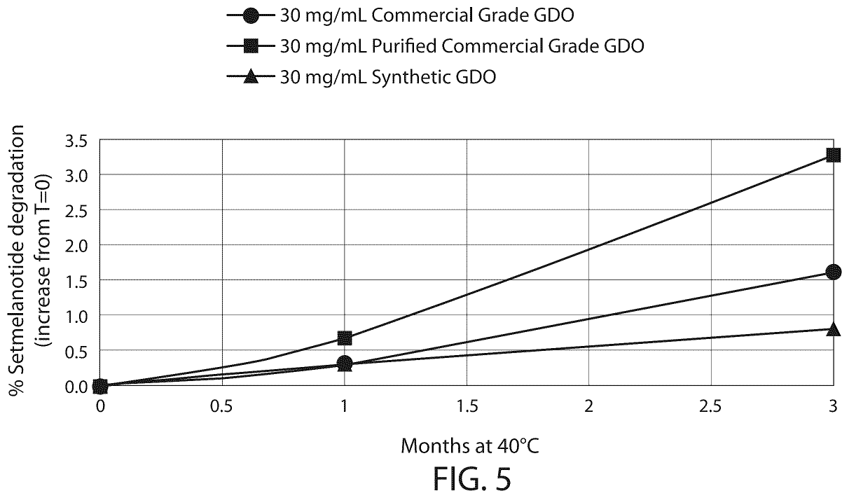

Components of exemplary setmelanotide formulations were varied in order to assess their impact on the stability of setmelanotide over time. In a first study, setmelanotide formulations were prepared as described in Example 2 with 30 mg/mL setmelanotide using different forms of GDO: 1) GDO of commercial purity; 2) GDO of commercial purity, which has been further purified to remove fatty acid compounds; and 3) synthetic GDO. The formulations were stored in 1 mL glass syringes equipped with staked stainless steel syringes and rubber plungers at 40° C. for up to three months. Samples were removed from each syringe over time and setmelanotide degradation was quantified by HPLC as shown in FIG. 5. As shown, formulations prepared with synthetic GDO exhibited a lower amount of setmelanotide degradation compared with that in the other formulations tested.

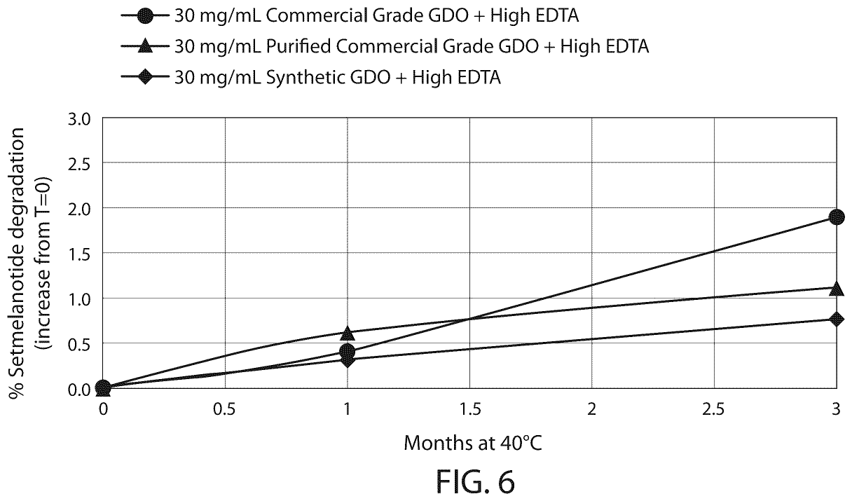

The effect of EDTA concentration on setmelanotide degradation was also investigated in a second study. Syringes containing setmelanotide formulations with higher EDTA along with GDO of varying sources were prepared, and setmelanotide degradation was quantified by HPLC over time. The results of this study are depicted in FIG. 6, and indicate that higher concentrations of EDTA have a small effect on setmelanotide stability in the presence of either GDO of commercial purity or synthetic GDO.

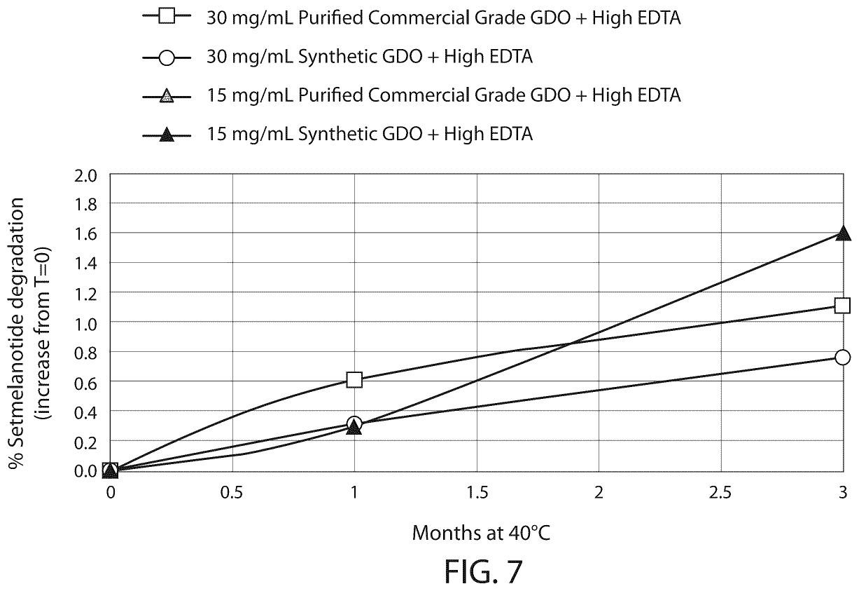

In a third study, the effect of setmelanotide and EDTA concentrations were explored in the presence of synthetic GDO. There, formulations containing 15 mg/mL setmelanotide exhibited higher levels of degradation compared with 30 mg/mL formulations, and increasing EDTA concentration appeared to have a greater effect as setmelanotide concentration decreases; see FIG. 7.

1. An injectable composition comprising:
a) glycerol dioleate;
b) phosphatidylcholinea;
c) an alcohol;
and
d) setmelanotide.

2. The injectable composition of claim 1, further comprising e) a polar solvent.

3-5. (canceled)

6. The injectable composition of claim 1, wherein the alcohol comprises ethanol.

7. The injectable composition of claim 6, wherein the ethanol is provided in an amount providing for a concentrations of setmelanotide of at least 10 mg/g, 20 mg/g or 30 mg/g.

8-16. (canceled)

17. The injectable compositions of claim 1, wherein the injectable composition is provided in a device.

18. The injectable composition of claim 17, wherein the device is chosen from manual syringe and an auto-injector.

19. The injectable composition of claim 2, wherein the polar solvent, comprises citrate buffer.

20. (canceled)

21. The injectable composition of claim 2, wherein the polar solvent comprises an additional component selected from an antioxidant, a chemical stabilizing agent and a physical stabilizing agent.

22. The injectable composition of claim 21, wherein the antioxidant is ethylene diamine (EDTA).

23. (canceled)

24. The injectable composition of claim 1, wherein the setmelanotide is present as a chloride salt.

25. The injectable composition of claim 1, further comprising an anti-microbial or microbial-static agent.

26. The injectable composition of claim 1, wherein the ratio, by weight, of a:b is 70:30 to 40:60.

27. (canceled)

28. The injectable composition of claim 1, wherein component a is 20-80%, 30-70%, 33-60%, or 38-43% by weight of the total weight of components a, b, and c in solution.

29-30. (canceled)

31. The injectable composition of claim 2, comprising:
glycerol dioleate;
phosphatidylcholine;
ethanol; and
a citrate buffer.

32. (canceled)

33. The injectable composition of claim 2, comprising, per one milliliter of composition:
420+/−20% mg glycerol dioleate (GDO);
420+/−20% mg soybean phosphatidylcholine;
105+/−20% mg ethanol;
20+/−20% mg citrate buffer; and
30+/−20% mg setmelanotide.

34-37. (canceled)

38. The injectable composition of claim 2, comprising:
glycerol dioleate of component a at 20-80%, 30-70%, 33-60%, or 38-43% by weight, of components a, b, and c; components a, b, c, and d; or components a, b, c, d, and e of the injectable composition;
phosphatidylcholine of component b at is 20-80%, 30-70%, 33-60%, or 38-43% by weight, of components a, b, and c; components a, b, c, and d; or components a, b, c, d, and e of the injectable composition;
alcohol of component c at 0.1-35%, 5-20%, 8-15%, or 9-11% by weight, of components a, b, and c; components a, b, c, and d; or components a, b, c, d, and e of the injectable composition;
polar solvent of component at 0.5-10%, 1-5%, or 1-3% by weight, of components a, b, and c; components a, b, c, and d; or components a, b, c, d, and e of the injectable composition; and
setmelanotide of component at 0.1-10%, 0.2-8%, 0.5-6%, 1-4% by weight, of components a, b, and c; components a, b, c, and d; or components a, b, c, d, and e of the injectable composition.

39-41. (canceled)

42. The injectable composition of claim 1, wherein when injected into a subject:
(i) an initial burst of setmelanotide, from the injectable composition after injection gives a ratio of maximum concentration (Cₘₐₓ) in plasma to minimum concentration (Cₘᵢₙ) in plasma before a next dose is administered, of less than 8; or
(ii) the injectable composition provides a low initial release of setmelanotide.

43-170. (canceled)
